(19) United States
(12) Patent Application Publication
(10) Pub. No.: US 2011/0319379 A1
(43) Pub. Date: Dec. 29, 2011

(54) Substituted Indazole Amides And Their Use As Glucokinase Activators

(76) Inventors: Jeffrey W. Corbett, Niantic, CT (US); Angel Guzman-Perez, Mystic, CT (US); Jeffrey A. Pfefferkorn, Mystic, CT (US); Meihua Tu, Mystic, CT (US)

(21) Appl. No.: 13/255,329

(22) PCT Filed: Mar. 4, 2010
(86) PCT No.: PCT/IB2010/050944

§ 371 Date: Sep. 8, 2011

Related U.S. Application Data
(60) Provisional application No. 61/159,101, filed on Mar. 11, 2009.
(60) Provisional application No. 61/299,515, filed on Jan. 29, 2010.

(51) Int. Cl. A61K 31/497; C07D 403/14; A61K 31/506; A61K 31/4439; C07D 403/12; A61K 31/416; C07D 401/12; C07D 413/14; A61K 31/5377; C07D 417/12; A61K 31/433; A61K 31/4192; C07D 409/14; A61P 3/04; A61P 3/10; C07D 401/14

(52) U.S. Cl. 514/210.18; 544/405; 514/255.05; 544/295; 546/275.7; 514/338; 548/361.1; 514/407; 544/120; 514/234.5; 548/128; 514/361; 548/255

(57) ABSTRACT

The present invention provides a compound of Formula (I) or a pharmaceutically acceptable salt thereof wherein R¹, R⁴, R⁶, X, Y and Z are as defined herein. The compounds of Formula (I) have been found to act as glucokinase activators. Consequently, the compounds of Formula (I) and the pharmaceutical compositions thereof are useful for the treatment of diseases, disorders, or conditions mediated by glucokinase.

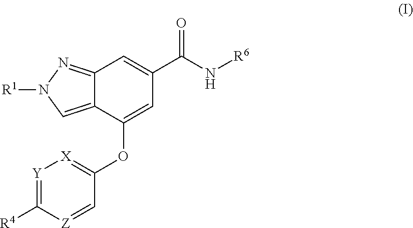

### FIELD OF THE INVENTION

The present invention relates to substituted indazole amides and the uses thereof as glucokinase activators.

### BACKGROUND

Diabetes is a major public health concern because of its increasing prevalence and associated health risks. The disease is characterized by metabolic defects in the production and utilization of carbohydrates which result in the failure to maintain appropriate blood glucose levels. Two major forms of diabetes are recognized. Type I diabetes, or insulin-dependent diabetes mellitus (IDDM), is the result of an absolute deficiency of insulin. Type II diabetes, or non-insulin dependent diabetes mellitus (NIDDM), often occurs with normal, or even elevated levels of insulin and appears to be the result of the inability of tissues and cells to respond appropriately to insulin. Aggressive control of NIDDM with medication is essential; otherwise it can progress into IDDM.

A promising area of diabetes research involves the use of small molecule allosteric activators of the glucokinase (GK) enzyme to lower blood glucose and normalize glucose stimulated insulin secretion Glucokinase is responsible for the conversion of glucose to glucose-6-phosphate (G-6-P), and it functions as a key regulator of glucose homeostasis. In the liver, GK regulates hepatic glucose utilization and output whereas in the pancreas it functions as a glucostat establishing the threshold for—cell glucose-stimulated insulin secretion. Glucokinase is also found in glucose sensing neurons of the ventromedial hypothalamus where it regulates the counter regulatory response (CRR) to hypoglycemia. Finally, glucokinase is reportedly expressed in the endocrine K and L cells where is may help regulate incretin release.

Therapeutically, it is anticipated that activation of glucokinase would be an effective strategy for lowering blood glucose by up regulating hepatic glucose utilization, down regulating hepatic glucose output and normalizing glucose stimulated insulin secretion. Consequently, a GK activator may provide therapeutic treatment for NIDDM and associated complications, inter alia, hyperglycemia, dyslipidemia, insulin resistance syndrome, hyperinsulinemia, hypertension, and obesity.

Several drugs in five major categories, each acting by different mechanisms, are available for treating hyperglycemia and subsequently, NIDDM (Moller, D. E., “New drug targets for Type 2 diabetes and the metabolic syndrome” Nature 414; 821-827, (2001)): (A) Insulin secretogogues, including sulphonyl-ureas (e.g., glipizide, glimepiride, glyburide) and meglitinides (e.g., nateglidine and repaglinide) enhance secretion of insulin by acting on the pancreatic beta-cells. While this therapy can decrease blood glucose level, it has limited efficacy and tolerability, causes weight gain and often induces hypoglycemia. (B) Biguanides (e.g., metformin) are thought to act primarily by decreasing hepatic glucose production. Biguanides often cause gastrointestinal disturbances and lactic acidosis, further limiting their use. (C) Inhibitors of alpha-glucosidase (e.g., acarbose) decrease intestinal glucose absorption. These agents often cause gastrointestinal disturbances. (D) Thiazolidinediones (e.g., pioglitazone, rosiglitazone) act on a specific receptor (peroxisome proliferator-activated receptor-gamma) in the liver, muscle and fat tissues. They regulate lipid metabolism subsequently enhancing the response of these tissues to the actions of insulin. Frequent use of these drugs may lead to weight gain and may induce edema and anemia. (E) Insulin is used in more severe cases, either alone or in combination with the above agents.

Ideally, an effective new treatment for NIDDM would meet the following criteria: (a) it would not have significant side effects including induction of hypoglycemia; (b) it would not cause weight gain; (c) it would at least partially replace insulin by acting via mechanism(s) that are independent from the actions of insulin; (d) it would desirably be metabolically stable to allow less frequent usage; and (e) it would be usable in combination with tolerable amounts of any of the categories of drugs listed herein.

Substituted heteroaryls, particularly pyridones, have been implicated in mediating GK and may play a significant role in the treatment of NIDDM. For example, U.S. Patent publication No. 2006/0058353 and PCT publication No's. WO2007/043638, WO2007/043638, and WO2007/117995 recite certain heterocyclic derivatives with utility for the treatment of diabetes. Although investigations are on-going, there still exists a need for a more effective and safe therapeutic treatment for diabetes, particularly NIDDM.

### SUMMARY

The present invention provides compounds of Formula (I) that act as glucokinase mediators, in particular, glucokinase activators; therefore, may be used in the treatment of diseases mediated by such activation (e.g., diseases related to Type 2 diabetes, and diabetes-related and obesity-related co-morbidities). The compounds are of Formula (I)

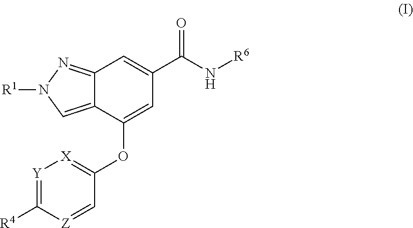

or a pharmaceutically acceptable salt thereof, wherein R¹ is (C₁-C₄)alkyl; X is C—R² or N, where R² is hydrogen, halo, or methyl; Y is C—R³ or N, where R³ is hydrogen, halo, or methyl; R⁴ is —CF₃, —SO₂R^{4a}, —C(O)NR^{4b}R^{4c}, —SO₂NR^{4b}R^{4c}, —N(R^{4b})SO₂R^{4a}, —N(R^{4b})C(O)NR^{4b}R^{4c} or —S(N)(O)R^{4b}; Z is C—R⁵ or N, where R⁵ is hydrogen, halo, or methyl; R⁶ is a 5- to 6-membered heteroaryl containing 1 to 3 nitrogen atoms optionally substituted with a substituent selected from the group consisting of (C₁-C₃)alkyl, (C₁-C₃)alkoxy, cyano, halo, —CO₂H, —CO₂(C₁-C₃)alkyl, —NH₂, —NH(C₁-C₄)alkyl, —N((C₁-C₃)alkyl)₂, —CF₃, —C(O)NH(C₁-C₃)alkyl, —C(O)N((C₁-C₃)alkyl)₂ and —C(R^{4b}R^{4c})C(O)N((C₁-C₃)alkyl)₂; R^{4a} is (C₁-C₃)alkyl or cyclopropyl; and R^{4b} and R^{4c} are at each occurrence independently (C₁-C₃)alkyl or hydrogen or taken together with the nitrogen to which they are attached form an azetidine, pyrrolidine or piperidine ring.

In one embodiment of the present invention, a compound of Formula (I) is provided wherein R¹ is methyl or ethyl; X is N or C—R², where R² is hydrogen or fluoro; Y is C—R³, where R³ is hydrogen or fluoro; R⁴ is —CF₃, —SO₂R^{4a},

—C(O)NR^{4b}R^{4c}, where R^{4a} is methyl, ethyl or cyclopropyl, and R^{4b} and Roc are both methyl or taken together forms a pyrrolidine ring; Z is N or C—R⁵, where R⁵ is hydrogen or fluoro; and R⁶ is a 5- to 6-membered heteroaryl selected from pyridin-2-yl, pyrazin-2-yl, 1H-pyrazol-3-yl, or 1,2,3-triazol-4-yl, wherein said heteroaryl is optionally substituted with methyl or methoxy.

In another embodiment of the present invention, a compound of Formula (I) is provided wherein R¹ is methyl or ethyl; X and Z are both N; Y is C—R³, where R³ is hydrogen or fluoro; R⁴ is —CF₃, —SO₂R^{4a}, —C(O)NR^{4b}R^{4c}, where R^{4a} is methyl, ethyl or cyclopropyl, and R^{4b} and R^{4c} are both methyl or taken together forms a pyrrolidine ring; Z is N or C—R⁵, where R⁵ is hydrogen or fluoro; and R⁶ is a 5- to 6-membered heteroaryl selected from pyridin-2-yl, pyrazin-2-yl, 1H-pyrazol-3-yl, or 1,2,3-triazol-4-yl, wherein said heteroaryl is optionally substituted with methyl or methoxy.

Preferred compounds of this embodiment include: 4-{[5-(dimethylcarbamoyl)pyrazin-2-yl]oxy}-2-methyl-N-pyrazin-2-yl-2H-indazole-6-carboxamide; 4-{[5-(dimethylcarbamoyl)pyrazin-2-yl]oxy}-2-methyl-N-(5-methylpyrazin-2-yl)-2H-indazole-6-carboxamide; 4-{[5-(dimethylcarbamoyl)pyrazin-2-yl]oxy}-2-methyl-N-(5-methylpyridin-2-yl)-2H-indazole-6-carboxamide; 4-{[5-(dimethylcarbamoyl)pyrazin-2-yl]oxy}-2-ethyl-N-(5-methylpyridin-2-yl)-2H-indazole-6-carboxamide; and 4-{[5-(dimethylcarbamoyl)pyrazin-2-yl]oxy}-2-ethyl-N-(5-methylpyrazin-2-yl)-2H-indazole-6-carboxamide.

In yet another embodiment of the present invention, a compound of Formula (I) is provided wherein R¹ is methyl or ethyl; X is C—R², where R² is hydrogen or fluoro; Y is C—R³, where R³ is hydrogen or fluoro; R⁴ is —CF₃, —SO₂R^{4a},

—C(O)NR^{4b}R^{4c}, where R^{4a} is methyl, ethyl or cyclopropyl, and R^{4b} and R^{4c} are both methyl or taken together forms a pyrrolidine ring; Z is C—R⁵, where R⁵ is hydrogen or fluoro; and R⁶ is a 5- to 6-membered heteroaryl selected from pyridin-2-yl, pyrazin-2-yl, 1H-pyrazol-3-yl, or 1,2,3-triazol-4-yl, wherein said heteroaryl is optionally substituted with methyl or methoxy.

Preferred compounds of this embodiment include: 2-methyl-N-(5-methylpyrazin-2-yl)-4-[4-(methylsulfonyl)phenoxy]-2H-indazole-6-carboxamide; 2-methyl-N-(5-methylpyridin-2-yl)-4-[4-(methylsulfonyl)phenoxy]-2H-indazole-6-carboxamide; 2-methyl-N-(1-methyl-1H-pyrazol-3-yl)-4-[4-(methylsulfonyl)phenoxy]-2H-indazole-6-carboxamide; 2-ethyl-4-[4-(methylsulfonyl)phenoxy]-N-pyridin-2-yl-2H-indazole-6-carboxamide; 2-ethyl-N-(5-methylpyridin-2-yl)-4-[4-(methylsulfonyl)phenoxy]-2H-indazole-6-carboxamide; 4-[4-(ethylsulfonyl)phenoxy]-2-methyl-N-pyridin-2-yl-2H-indazole-6-carboxamide; 4-[4-(ethylsulfonyl)phenoxy]-2-methyl-N-(5-methylpyrazin-2-yl)-2H-indazole-6-carboxamide; and 2-ethyl-4-[4-(ethylsulfonyl)phenoxy]-N-(5-methylpyridin-2-yl)-2H-indazole-6-carboxamide.

In yet another embodiment of the present invention, a compound of Formula (I) is provided wherein R¹ is methyl or ethyl; X is C—R², where R² is hydrogen; Y and Z are both N; R⁴ is —CF₃, —SO₂R^{4a}, —C(O)NR^{4b}R^{4c}, where R^{4a} is methyl, ethyl or cyclopropyl, and R^{4b} and R^{4c} are both methyl or taken together forms a pyrrolidine ring; and R⁶ is a 5- to 6-membered heteroaryl selected from pyridin-2-yl, pyrazin-2-yl, 1H-pyrazol-3-yl, or 1,2,3-triazol-4-yl, wherein said heteroaryl is optionally substituted with methyl or methoxy.

Preferred compounds of this embodiment include: 4-{[2-(dimethylcarbamoyl)pyrimidin-5-yl]oxy}-2-methyl-N-(5-methylpyridin-2-yl)-2H-indazole-6-carboxamide; and N-(5-chloropyridin-2-yl)-4-(2-(dimethylcarbamoyl)pyrimidin-5-yloxy)-2-methyl-2H-indazole-6-carboxamide.

In yet another embodiment of the present invention, a compound of Formula (I) is provided wherein R¹ is methyl or ethyl; X is C—R², where R² is hydrogen or fluoro; Y is C—R³, where R³ is hydrogen or fluoro; R⁴ is —CF₃, —SO₂R^{4a}, —C(O)NR^{4b}R^{4c}, where R^{4a} is methyl, ethyl or cyclopropyl, and Rob and R^{4c} are both methyl or taken together forms a pyrrolidine ring; Z is N; and R⁶ is a 5- to 6-membered heteroaryl selected from pyridin-2-yl, pyrazin-2-yl, 1H-pyrazol-3-yl, or 1,2,3-triazol-4-yl, wherein said heteroaryl is optionally substituted with methyl or methoxy.

Preferred compounds of this embodiment include: 4-{[6-(dimethylcarbamoyl)-5-fluoropyridin-3-yl]oxy}-2-methyl-N-(5-methylpyrazin-2-yl)-2H-indazole-6-carboxamide; and 4-(6-(dimethylcarbamoyl)-5-fluoropyridin-3-yloxy)-2-ethyl-N-(5-methylpyrazin-2-yl)-2H-indazole-6-carboxamide.

Another embodiment of the present invention is a compound selected from 4-(6-(azetidine-1-carbonyl)-5-fluoropyridin-3-yloxy)-2-ethyl-N-(5-methylpyrazin-2-yl)-2H-indazole-6-carboxamide; N-(5-ethoxypyrazin-2-yl)-2-ethyl-4-[4-(methylsulfonyl)phenoxy]-2H-indazole-6-carboxamide; 2-ethyl-N-(5-ethylpyrazin-2-yl)-4-[4-(methylsulfonyl)phenoxy]-2H-indazole-6-carboxamide; 4-[4-(aminosulfonyl)phenoxy]-N-(5-ethoxypyrazin-2-yl)-2-ethyl-2H-indazole-6-carboxamide; and 2-ethyl-N-(5-methylpyrazin-2-yl)-4-[4-(methylsulfonyl)phenoxy]-2H-indazole-6-carboxamide; or a pharmaceutically acceptable salt thereof.

Another aspect of the present invention is a pharmaceutical composition that comprises (1) a compound of the present invention, and (2) a pharmaceutically acceptable excipient, diluent, or carrier. Preferably, the composition comprises a therapeutically effective amount of a compound of the present invention. The composition may also contain at least one additional pharmaceutical agent (described herein). Preferred agents include anti-obesity agents and/or anti-diabetic agents (described herein below).

In yet another aspect of the present invention is a method for treating a disease, condition, or disorder mediated by glucokinase, in particular, activation of said enzyme, in a mammal that includes the step of administering to a mammal, preferably a human, in need of such treatment a therapeutically effective amount of a compound of the present invention, or a pharmaceutical composition thereof.

Diseases, disorders, or conditions mediated by glucokinase activators include Type II diabetes, hyperglycemia, metabolic syndrome, impaired glucose tolerance, glucosuria, cataracts, diabetic neuropathy, diabetic nephropathy, diabetic retinopathy, obesity, dyslididemia, hypertension, hyperinsulinemia, and insulin resistance syndrome. Preferred diseases, disorders, or conditions include Type II diabetes, hyperglycemia, impaired glucose tolerance, obesity, and insulin resistance syndrome. More preferred are Type II diabetes, hyperglycemia, and obesity. Most preferred is Type II diabetes.

In yet another aspect of the present invention is a method of reducing the level of blood glucose in a mammal, preferably a human, which includes the step of administering to a mammal in need of such treatment a therapeutically effective amount of a compound of the present invention, or a pharmaceutical composition thereof.

Compounds of the present invention may be administered in combination with other pharmaceutical agents (in particular, anti-obesity and anti-diabetic agents described herein below). The combination therapy may be administered as (a) a single pharmaceutical composition which comprises a compound of the present invention, at least one additional pharmaceutical agent described herein and a pharmaceutically acceptable excipient, diluent, or carrier; or (b) two separate pharmaceutical compositions comprising (i) a first composition comprising a compound of the present invention and a pharmaceutically acceptable excipient, diluent, or carrier, and (ii) a second composition comprising at least one additional pharmaceutical agent described herein and a pharmaceutically acceptable excipient, diluent, or carrier. The pharmaceutical compositions may be administered simultaneously or sequentially and in any order.

### DEFINITIONS

As used herein, the term “alkyl” refers to a hydrocarbon radical of the general formula CₙH₂ₙ₊₁. The alkane radical may be straight or branched. For example, the term “(C₁-C₆)alkyl” refers to a monovalent, straight, or branched aliphatic group containing 1 to 6 carbon atoms (e.g., methyl, ethyl, n-propyl, i-propyl, n-butyl, i-butyl, s-butyl, t-butyl, n-pentyl, 1-methylbutyl, 2-methylbutyl, 3-methylbutyl, neopentyl, 3,3-dimethylpropyl, hexyl, 2-methylpentyl, and the like). Similarly, the alkyl portion (i.e., alkyl moiety) of an alkoxy, acyl (e.g., alkanoyl), alkylamino, dialkylamino, alkylsulfonyl, and alkylthio group have the same definition as above. When indicated as being “optionally substituted”, the alkane radical or alkyl moiety may be unsubstituted or substituted with one or more substituents (generally, one to three substituents except in the case of halogen substituents such as perchloro or perfluoroalkyls). “Halo-substituted alkyl” refers to an alkyl group substituted with one or more halogen atoms (e.g., fluoromethyl, difluoromethyl, trifluoromethyl, perfluoroethyl, 1,1-difluoroethyl and the like).

The term “cycloalkyl” refers to nonaromatic rings that are fully hydrogenated and may exist as a single ring, bicyclic ring or a spiral ring. Unless specified otherwise, the carbocyclic ring is generally a 3- to 8-membered ring. For example, cycloalkyl include groups such as cyclopropyl, cyclobutyl, cyclopentyl, cyclohexyl, cyclohexenyl, norbornyl (bicyclo[2.2.1]heptyl), bicyclo[2.2.2]octyl, and the like.

The term “heterocycle” refers to nonaromatic rings that are fully hydrogenated and may exist as a single ring, bicyclic ring or a spiral ring. Unless specified otherwise, the heterocyclic ring is generally a 3- to 6-membered ring containing 1 to 3 heteroatoms (preferably 1 or 2 heteroatoms) independently selected from sulfur, oxygen and/or nitrogen. Heterocyclic rings include groups such as epoxy, aziridinyl, tetrahydrofuranyl, pyrrolidinyl, N-methylpyrrolidinyl, piperidinyl, piperazinyl, pyrazolidinyl, 4H-pyranyl, morpholino, thiomorpholino, tetrahydrothienyl, tetrahydrothienyl 1,1-dioxide, and the like.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “animal” refers to humans (male or female), companion animals (e.g., dogs, cats and horses), food-source animals, zoo animals, marine animals, birds and other similar animal species. “Edible animals” refers to food-source animals such as cows, pigs, sheep and poultry.

The phrase “pharmaceutically acceptable” indicates that the substance or composition must be compatible chemically and/or toxicologically, with the other ingredients comprising a formulation, and/or the mammal being treated therewith.

The terms “treating”, “treat”, or “treatment” embrace both preventative, i.e., prophylactic, and palliative treatment.

The terms “modulated” or “modulating”, or “modulate(s)”, as used herein, unless otherwise indicated, refers to the activation of the activating the glucokinase enzyme with compounds of the present invention.

The terms “mediated” or “mediating” or “mediate(s)”, as used herein, unless otherwise indicated, refers to the treatment or prevention the particular disease, condition, or disorder, (ii) attenuation, amelioration, or elimination of one or more symptoms of the particular disease, condition, or disorder, or (iii) prevention or delay of the onset of one or more symptoms of the particular disease, condition, or disorder described herein, by activating the glucokinase enzyme via glucose binding enhancement, alleviating the inhibition of glucokinase regulatory protein, a key regulator of glucokinase activity in the liver, and/or by increasing the catalytic rate of the glucokinase enzyme (e.g., change Vmax).

The term “compounds of the present invention” (unless specifically identified otherwise) refer to compounds of Formula (I) and any pharmaceutically acceptable salts of the compounds, as well as, all stereoisomers (including diastereoisomers and enantiomers), tautomers, conformational isomers, and isotopically labeled compounds. Hydrates and solvates of the compounds of the present invention are considered compositions of the present invention, wherein the compound is in association with water or solvent, respectively.

### DETAILED DESCRIPTION

Compounds of the present invention may be synthesized by synthetic routes that include processes analogous to those well-known in the chemical arts, particularly in light of the description contained herein. The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

For illustrative purposes, the reaction schemes depicted below provide potential routes for synthesizing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

In the preparation of compounds of the present invention, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (BOC), benzyloxycarbonyl (CBz) and 9-fluorenylmethyleneoxycarbonyl (Fmoc). Similarly, a “hydroxy-protecting group” refers to a substituent of a hydroxy group that blocks or protects the hydroxy functionality. Suitable hydroxyl-protecting groups (O-Pg) include for example, allyl, acetyl, silyl, benzyl, para-methoxybenzyl, trityl, and the like. The need for such protection is readily determined by one skilled in the art.

For a general description of protecting groups and their use, see T. W. Greene, Protective Groups in Organic Synthesis, John Wiley & Sons, New York, 1991.

Scheme I outlines the general procedures one could use to provide compounds of the present invention having Formula (I).

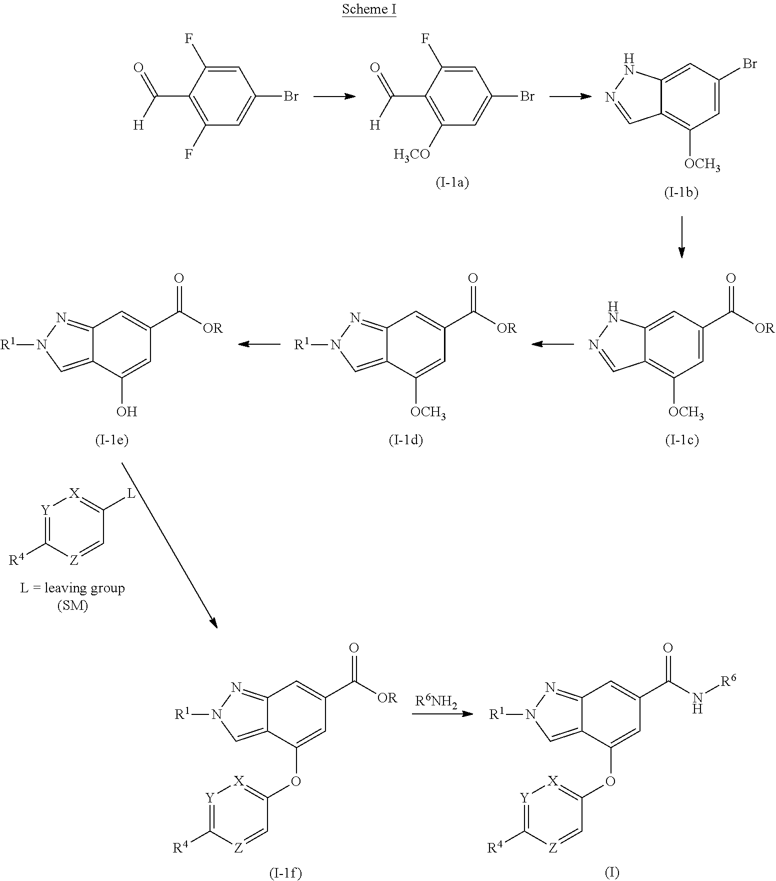

The methoxydihalosubstituted aldehyde (I-1a) can be prepared from the corresponding trihalosubstituted aldehyde by reacting with an alkali metal methoxide in methanol under an inert atmosphere followed by heating to reflux. The indazole intermediate (I-1b) can then be prepared by reacting the methoxydihalsubstitued aldehyde (I-1a) with hydrazine monohydrate under elevated temperatures. The bromo group on the indazole intermediate (I-1b) can be converted to the corresponding carboxylate ester using a palladium-mediated carbonylative coupling reaction (see Tsuji, J. Palladium Reagents and Catalysts, Wiley, New York (2004)). For example, treatment of indazole intermediate (I-1b) with 1,3-bis(diphenylphosphino)propane, and palladium (II) acetate in the presence of a base (e.g., triethylamine) under pressure with carbon monoxide affords ester intermediate (I-1c). The desired R¹ group can be added to the indazole carboxylate intermediate (I-1c) using the appropriate trialkyloxonium tetrafluoroborate (e.g., trimethyloxonium tetrafluoroborate for R¹=methyl and triethyloxonium tetrafluoroborate for R¹=ethyl). The methoxy group can then be converted to a hydroxyl group using standard methods well-known to those of skill in the art. For example, intermediate (I-1d) can be treated with boron tribromide. Alternatively, the methoxy protecting group can be removed using various other reagents known to those skilled in the art including: trimethylsilyl iodide, aluminum tribromide or sodium in liquid ammonia. Additionally, other protecting groups beyond the methyl ether can also be utilized in this reaction sequence. Non-limiting examples of alternative protecting groups include: benzyl ether (Bn), tert-butylsimethylsilyl ether (TBS) or methoxymethyl ether (MOM). Once the hydroxyl group is free, then the desired heteroaryl group (SM) can be attached via a substitution reaction. For example, intermediate (I-1e) can be treated with cesium carbonate and copper iodide in a basic solvent (e.g., N,N-dimethylformamide (DMF)) in the presence of the desired heteroaryl group having a leaving group (e.g., halo, mesylate, tosylate, etc.) at elevated temperatures to form intermediate (I-1f). The final product (I) can then be formed by using standard amide (or peptide) formation procedures using the desired amine (R⁶NH₂).

The compounds of the present invention may be isolated and used per se, or when possible, in the form of its pharmaceutically acceptable salt. The term “salts” refers to inorganic and organic salts of a compound of the present invention. These salts can be prepared in situ during the final isolation and purification of a compound, or by separately reacting the compound with a suitable organic or inorganic acid or base and isolating the salt thus formed. Representative salts include the hydrobromide, hydrochloride, hydroiodide, sulfate, bisulfate, nitrate, acetate, trifluoroacetate, oxalate, besylate, palmitiate, pamoate, malonate, stearate, laurate, malate, borate, benzoate, lactate, phosphate, hexafluorophosphate, benzene sulfonate, tosylate, formate, citrate, maleate, fumarate, succinate, tartrate, naphthylate, mesylate, glucoheptonate, lactobionate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. See, e.g., Berge, et al., J. Pharm. Sci., 66, 1-19 (1977).

The compounds of the present invention may contain asymmetric or chiral centers, and, therefore, exist in different stereoisomeric forms. Unless specified otherwise, it is intended that all stereoisomeric forms of the compounds of the present invention as well as mixtures thereof, including racemic mixtures, form part of the present invention. In addition, the present invention embraces all geometric and positional isomers. For example, if a compound of the present invention incorporates a double bond or a fused ring, both the cis- and trans-forms, as well as mixtures, are embraced within the scope of the invention.

Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Also, some of the compounds of the present invention may be atropisomers (e.g., substituted biaryls) and are considered as part of this invention. Enantiomers can also be separated by use of a chiral HPLC column. Alternatively, the specific stereoisomers may be synthesized by using an optically active starting material, by asymmetric synthesis using optically active reagents, substrates, catalysts or solvents, or by converting one stereoisomer into the other by asymmetric transformation.

It is also possible that the intermediates and compounds of the present invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

Certain compounds of the present invention may exist in different stable conformational forms which may be separable. Torsional asymmetry due to restricted rotation about an asymmetric single bond, for example, because of steric hindrance or ring strain, may permit separation of different conformers.

The present invention also embraces isotopically-labeled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, sulfur, fluorine, iodine, and chlorine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ¹²³I, ¹²⁵I and ³⁶Cl, respectively.

Certain isotopically-labeled compounds of the present invention (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C, and ¹⁸F are useful for positron emission tomography (PET) studies to examine substrate occupancy. Isotopically labeled compounds of the present invention can generally be prepared by following procedures analogous to those disclosed in the Schemes and/or in the Examples herein below, by substituting an isotopically labeled reagent for a non-isotopically labeled reagent.

Certain compounds of the present invention may exist in more than one crystal form (generally referred to as “polymorphs”). Polymorphs may be prepared by crystallization under various conditions, for example, using different solvents or different solvent mixtures for recrystallization; crystallization at different temperatures; and/or various modes of cooling, ranging from very fast to very slow cooling during crystallization. Polymorphs may also be obtained by heating or melting the compound of the present invention followed by gradual or fast cooling. The presence of polymorphs may be determined by solid probe NMR spectroscopy, IR spectroscopy, differential scanning calorimetry, powder X-ray diffraction or such other techniques.

Compounds of the present invention are useful for treating diseases, conditions and/or disorders modulated by the activation of the glucokinase enzyme; therefore, another embodiment of the present invention is a pharmaceutical composition comprising a therapeutically effective amount of a compound of the present invention and a pharmaceutically acceptable excipient, diluent or carrier. The compounds of the present invention (including the compositions and processes used therein) may also be used in the manufacture of a medicament for the therapeutic applications described herein.

A typical formulation is prepared by mixing a compound of the present invention and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament).

The formulations may be prepared using conventional dissolution and mixing procedures. For example, the bulk drug substance (i.e., compound of the present invention or stabilized form of the compound (e.g., complex with a cyclodextrin derivative or other known complexation agent)) is dissolved in a suitable solvent in the presence of one or more of the excipients described above. The compound of the present invention is typically formulated into pharmaceutical dosage forms to provide an easily controllable dosage of the drug and to give the patient an elegant and easily handleable product.

The pharmaceutical compositions also include solvates and hydrates of the compounds of Formula (I). The term “solvate” refers to a molecular complex of a compound represented by Formula (I) (including pharmaceutically acceptable salts thereof) with one or more solvent molecules. Such solvent molecules are those commonly used in the pharmaceutical art, which are known to be innocuous to the recipient, e.g., water, ethanol, ethylene glycol, and the like. The term “hydrate” refers to the complex where the solvent molecule is water. The solvates and/or hydrates preferably exist in crystalline form. Other solvents may be used as intermediate solvates in the preparation of more desirable solvates, such as methanol, methyl t-butyl ether, ethyl acetate, methyl acetate, (S)-propylene glycol, (R)-propylene glycol, 1,4-butyne-diol, and the like.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The present invention further provides a method of treating diseases, conditions and/or disorders modulated by the activation of the glucokinase enzyme in an animal that includes administering to an animal in need of such treatment a therapeutically effective amount of a compound of the present invention or a pharmaceutical composition comprising an effective amount of a compound of the present invention and a pharmaceutically acceptable excipient, diluent, or carrier. The method is particularly useful for treating diseases, conditions and/or disorders that benefit from the activation of glucokinase which include: eating disorders (e.g., binge eating disorder, anorexia, bulimia, weight loss or control and obesity), prevention of obesity and insulin resistance by glucokinase expression in skeletal muscle of transgenic mice (Otaegui, P. J., et. al., The FASEB Journal, 17; 2097-2099, (2003)); and Type II diabetes, insulin resistance syndrome, insulin resistance, and hyperglycemia (Poitout, V., et. al., “An integrated view of J3-cell dysfunction in type-II diabetes”, Annul. Rev. Medicine, 47; 69-83, (1996)).

One aspect of the present invention is the treatment of obesity, and obesity-related disorders (e.g., overweight, weight gain, or weight maintenance).

Obesity and overweight are generally defined by body mass index (BMI), which is correlated with total body fat and estimates the relative risk of disease. BMI is calculated by weight in kilograms divided by height in meters squared (kg/m²). Overweight is typically defined as a BMI of 25-29.9 kg/m², and obesity is typically defined as a BMI of 30 kg/m². See, e.g., National Heart, Lung, and Blood Institute, Clinical Guidelines on the Identification, Evaluation, and Treatment of Overweight and Obesity in Adults, The Evidence Report, Washington, D.C.: U.S. Department of Health and Human Services, NIH publication no. 98-4083 (1998).

Another aspect of the present invention is for the treatment or delaying the progression or onset of diabetes or diabetes-related disorders including Type 1 (insulin-dependent diabetes mellitus, also referred to as “IDDM”) and Type 2 (noninsulin-dependent diabetes mellitus, also referred to as “NIDDM”) diabetes, impaired glucose tolerance, insulin resistance, hyperglycemia, and diabetic complications (such as atherosclerosis, coronary heart disease, stroke, peripheral vascular disease, nephropathy, hypertension, neuropathy, and retinopathy).

Yet another aspect of the present invention is the treatment of diabetes- or obesity-related co-morbidities, such as metabolic syndrome. Metabolic syndrome includes diseases, conditions or disorders such as dyslipidemia, hypertension, insulin resistance, diabetes (e.g., Type 2 diabetes), weight gain, coronary artery disease and heart failure. For more detailed information on Metabolic Syndrome, see, e.g., Zimmet, P. Z., et al., “The Metabolic Syndrome: Perhaps an Etiologic Mystery but Far From a Myth—Where Does the International Diabetes Federation Stand?,” Diabetes & Endocrinology, 7(2), (2005); and Alberti, K. G., et al., “The Metabolic Syndrome—A New Worldwide Definition,” Lancet, 366, 1059-62 (2005). Preferably, administration of the compounds of the present invention provides a statistically significant (p<0.05) reduction in at least one cardiovascular disease risk factor, such as lowering of plasma leptin, C-reactive protein (CRP) and/or cholesterol, as compared to a vehicle control containing no drug. The administration of compounds of the present invention may also provide a statistically significant (p<0.05) reduction in glucose serum levels.

In yet another aspect of the present invention, the condition treated is impaired glucose tolerance, hyperglycemia, diabetic complications such as sugar cataracts, diabetic neuropathy, diabetic nephropathy, diabetic retinopathy and diabetic cardiomyopathy, anorexia nervosa, bulimia, cachexia, hyperuricemia, hyperinsulinemia, hypercholesterolemia, hyperlipidemia, dyslipidemia, mixed dyslipidemia, hypertriglyceridemia, nonalcoholic fatty liver disease, atherosclerosis, arteriosclerosis, acute heart failure, congestive heart failure, coronary artery disease, cardiomyopathy, myocardial infarction, angina pectoris, hypertension, hypotension, stroke, ischemia, ischemic reperfusion injury, aneurysm, restenosis, vascular stenosis, solid tumors, skin cancer, melanoma, lymphoma, breast cancer, lung cancer, colorectal cancer, stomach cancer, esophageal cancer, pancreatic cancer, prostate cancer, kidney cancer, liver cancer, bladder cancer, cervical cancer, uterine cancer, testicular cancer and ovarian cancer.

The present invention also relates to therapeutic methods for treating the above described conditions in a mammal, including a human, wherein a compound of formula (I) of this invention is administered as part of an appropriate dosage regimen designed to obtain the benefits of the therapy. The appropriate dosage regimen, the amount of each dose administered and the intervals between doses of the compound will depend upon the compound of formula (I) of this invention being used, the type of pharmaceutical compositions being used, the characteristics of the subject being treated and the severity of the conditions.

In general, an effective dosage for the compounds of the present invention is in the range of 0.01 mg/kg/day to 30 mg/kg/day, preferably 0.01 mg/kg/day to 5 mg/kg/day of active compound in single or divided doses. However, some variability in the general dosage range may be required depending upon the age and weight of the subject being treated, the intended route of administration, the particular compound being administered and the like. The determination of dosage ranges and optimal dosages for a particular patient is well within the ability of one of ordinary skill in the art having the benefit of the instant disclosure. Practitioners will appreciate that “kg” refers to the weight of the patient measured in kilograms.

The compounds or compositions of this invention may be administered in single (e.g., once daily) or multiple doses or via constant infusion. The compounds of this invention may also be administered alone or in combination with pharmaceutically acceptable carriers, vehicles or diluents, in either single or multiple doses. Suitable pharmaceutical carriers, vehicles and diluents include inert solid diluents or fillers, sterile aqueous solutions and various organic solvents.

The compounds or compositions of the present invention may be administered to a subject in need of treatment by a variety of conventional routes of administration, including orally and parenterally, (e.g., intravenously, subcutaneously or intramedullary). Further, the pharmaceutical compositions of this invention may be administered intranasally, as a suppository, or using a “flash” formulation, i.e., allowing the medication to dissolve in the mouth without the need to use water.

It is also noted that the compounds of the present invention can be used in sustained release, controlled release, and delayed release formulations, which forms are also well known to one of ordinary skill in the art.

The compounds of this invention may also be used in conjunction with other pharmaceutical agents for the treatment of the diseases, conditions and/or disorders described herein. Therefore, methods of treatment that include administering compounds of the present invention in combination with other pharmaceutical agents are also provided. Suitable pharmaceutical agents that may be used in combination with the compounds of the present invention include anti-obesity agents (including appetite suppressants), anti-diabetic agents, anti-hyperglycemic agents, lipid lowering agents, and anti-hypertensive agents.

Suitable anti-diabetic agents include an acetyl-CoA carboxylase-2 (ACC-2) inhibitor, a diacylglycerol O-acyltransferase 1 (DGAT-1) inhibitor, a phosphodiesterase (PDE)-10 inhibitor, a sulfonylurea (e.g., acetohexamide, chlorpropamide, diabinese, glibenclamide, glipizide, glyburide, glimepiride, gliclazide, glipentide, gliquidone, glisolamide, tolazamide, and tolbutamide), a meglitinide, an α-amylase inhibitor (e.g., tendamistat, trestatin and AL-3688), an α-glucoside hydrolase inhibitor (e.g., acarbose), an α-glucosidase inhibitor (e.g., adiposine, camiglibose, emiglitate, miglitol, voglibose, pradimicin-Q, and salbostatin), a PPARγ agonist (e.g., balaglitazone, ciglitazone, darglitazone, englitazone, isaglitazone, pioglitazone, rosiglitazone and troglitazone), a PPAR α/γ agonist (e.g., CLX-0940, GW-1536, GW-1929, GW-2433, KRP-297, L-796449, LR-90, MK-0767 and SB-219994), a biguanide (e.g., metformin), a glucagon-like peptide 1 (GLP-1) agonist (e.g., exendin-3 and exendin-4), a protein tyrosine phosphatase-1B (PTP-1B) inhibitor (e.g., trodusquemine, hyrtiosal extract, and compounds disclosed by Zhang, S., et al., Drug Discovery Today, 12(9/10), 373-381 (2007)), SIRT-1 inhibitor (e.g., reservatrol), a dipeptidyl peptidease IV (DPP-IV) inhibitor (e.g., sitagliptin, vildagliptin, alogliptin and saxagliptin), an insulin secreatagogue, a fatty acid oxidation inhibitor, an A2 antagonist, a c-jun amino-terminal kinase (JNK) inhibitor, insulin, an insulin mimetic, a glycogen phosphorylase inhibitor, and a VPAC2 receptor agonist. Preferred anti-diabetic agents are metformin and DPP-IV inhibitors (e.g., sitagliptin, vildagliptin, alogliptin and saxagliptin).

Suitable anti-obesity agents include 11β-hydroxy steroid dehydrogenase-1 (11β-HSD type 1) inhibitors, stearoyl-CoA desaturase-1 (SCD-1) inhibitor, MCR-4 agonists, cholecystokinin-A (CCK-A) agonists, monoamine reuptake inhibitors (such as sibutramine), sympathomimetic agents, β₃ adrenergic agonists, dopamine agonists (such as bromocriptine), melanocyte-stimulating hormone analogs, 5HT2c agonists, melanin concentrating hormone antagonists, leptin (the OB protein), leptin analogs, leptin agonists, galanin antagonists, lipase inhibitors (such as tetrahydrolipstatin, i.e. orlistat), anorectic agents (such as a bombesin agonist), neuropeptide-γ antagonists (e.g., NPY Y5 antagonists), PYY₃₋₃₆ (including analogs thereof), thyromimetic agents, dehydroepiandrosterone or an analog thereof, glucocorticoid agonists or antagonists, orexin antagonists, glucagon-like peptide-1 agonists, ciliary neurotrophic factors (such as Axokine™ available from Regeneron Pharmaceuticals, Inc., Tarrytown, N.Y. and Procter & Gamble Company, Cincinnati, Ohio), human agouti-related protein (AGRP) inhibitors, ghrelin antagonists, histamine 3 antagonists or inverse agonists, neuromedin U agonists, MTP/ApoB inhibitors (e.g., gut-selective MTP inhibitors, such as dirlotapide), opioid antagonist, orexin antagonist, and the like.

Preferred anti-obesity agents for use in the combination aspects of the present invention include gut-selective MTP inhibitors (e.g., dirlotapide, mitratapide and implitapide, R56918 (CAS No. 403987) and CAS No. 913541-47-6), CCKa agonists (e.g., N-benzyl-2-[4-(1H-indol-3-ylmethyl)-5-oxo-1-phenyl-4,5-dihydro-2,3,6,10b-tetraaza-benzo[e]azulen-6-yl]-N-isopropyl-acetamide described in PCT Publication No. WO 2005/116034 or US Publication No. 2005-0267100 A1), 5HT2c agonists (e.g., lorcaserin), MCR4 agonist (e.g., compounds described in U.S. Pat. No. 6,818,658), lipase inhibitor (e.g., Cetilistat), PYY₃₋₃₆ (as used herein “PYY₃₋₃₆” includes analogs, such as peglated PYY₃₋₃₆ e.g., those described in US Publication 2006/0178501), opioid antagonists (e.g., naltrexone), oleoyl-estrone (CAS No. 180003-17-2), obinepitide (TM30338), pramlintide (Symlin®), tesofensine (NS2330), leptin, liraglutide, bromocriptine, orlistat, exenatide (Byetta®), AOD-9604 (CAS No. 221231-10-3) and sibutramine. Preferably, compounds of the present invention and combination therapies are administered in conjunction with exercise and a sensible diet.

All of the above recited U.S. patents and publications are incorporated herein by reference.

Embodiments of the present invention are illustrated by the following Examples. It is to be understood, however, that the embodiments of the invention are not limited to the specific details of these Examples, as other variations thereof will be known, or apparent in light of the instant disclosure, to one of ordinary skill in the art.

### EXAMPLES

Unless specified otherwise, starting materials are generally available from commercial sources such as Aldrich Chemicals Co. (Milwaukee, Wis.), Lancaster Synthesis, Inc. (Windham, N.H.), Acros Organics (Fairlawn, N.J.), Maybridge Chemical Company, Ltd. (Cornwall, England), Tyger Scientific (Princeton, N.J.), and AstraZeneca Pharmaceuticals (London, England).

### General Experimental Procedures

NMR spectra were recorded on a Varian Unity™ 400 (available from Varian Inc., Palo Alto, Calif.) at room temperature at 400 MHz for proton. Chemical shifts are expressed in parts per million (δ) relative to residual solvent as an internal reference. The peak shapes are denoted as follows: s, singlet; d, doublet; dd, doublet of doublet; t, triplet; q, quartet; m, multiplet; bs, broad singlet; 2s, two singlets. Atmospheric pressure chemical ionization mass spectra (APCI) were obtained on a Fisons™ Platform II Spectrometer (carrier gas: acetonitrile: available from Micromass Ltd, Manchester, UK). Chemical ionization mass spectra (CI) were obtained on a Hewlett-Packard™5989 instrument (ammonia ionization, PBMS: available from Hewlett-Packard Company, Palo Alto, Calif.). Electrospray ionization mass spectra (ES) were obtained on a Waters™ ZMD instrument (carrier gas: acetonitrile: available from Waters Corp., Milford, Mass.). High resolution mass spectra (HRMS) were obtained on an Agilent™ Model 6210 using time of flight method. Where the intensity of chlorine or bromine-containing ions are described, the expected intensity ratio was observed (approximately 3:1 for ³⁵Cl/³⁷Cl containing ions and 1:1 for ⁷⁹Br/⁸¹Br-containing ions) and the intensity of only the lower mass ion is given. In some cases only representative ¹H NMR peaks are given. Optical rotations were determined on a PerkinElmer™ 241 polarimeter (available from PerkinElmer Inc., Wellesley, Mass.) using the sodium D line (λ=589 nm) at the indicated temperature and are reported as follows [α]_{D}^{temp}, concentration (c=g/100 ml), and solvent.

Column chromatography was performed with either Baker™ silica gel (40 μm; J. T. Baker, Phillipsburg, N.J.) or Silica Gel 50 (EM Sciences™ Gibbstown, N.J.) in glass columns or in Flash 40 Biotage™ columns (ISC, Inc., Shelton, Conn.) or Biotage™ SNAP cartridge KPsil or Redisep Rf silica (from Teledyne™ Isco™) under low nitrogen pressure.

### Starting Materials

The following starting materials are available from the corresponding sources;
4-bromo-2,6-difluorobenzaldehyde—Alfa Aesar (Ward Hill, Mass.)
5-chloropyrazine-2-carboxylic acid—Ark Pharma, Inc. (Libertyville, Ill.)
2-chloro-5-(trifluoromethyl)pyrazine—Anichem (Monmouth Junction, N.J.)
1-methyl-1H-1,2,3-triazol-4-amine—ChemBridge Corporation (San Diego, Calif.)
4-fluoro-N,N-dimethylbenzenesulfonamide—Combi-Blocks Inc. (San Diego, Calif.)
4-fluoro-N-methylbenzenesulfonamide—Combi-Blocks Inc. (San Diego, Calif.)
bis(2,4-dimethoxybenzyl)amine—3B Scientific Corporation (Libertyville, Ill.)
N-methyl-ethanesulfonamide—Aurora Fine Chemicals LLC (San Diego, Calif.)
2,5-dichloropyrazine—Ark Pharma, Inc (Libertyville, Ill.)
2-chloro-5-(methylsulfonyl)benzotrifluoride—ACC Corp. (San Diego, Calif.)
5-bromo-2-methylsulfonyl pyridine—ACC Corp. (San Diego, Calif.)
2-(3-amino-1H-pyrazol-1-yl)acetamide—Enamine, Ltd. (Kiev, Ukraine)
2-amino-5-methoxypyrazine—Ark Pharma, Inc. (Libertyville, Ill.)
2-Amino-5-ethoxypyrazine—Ark Pharma, Inc. (Libertyville, Ill.)
5-ethylpyrazin-2-amine—Anichem (Monmouth Junction, N.J.)
1-methyl-1H-pyrazol-3-amine—Fluorochem (West Columbia, S.C.)
5-methylpyrazin-2-amine—Anichem (Monmouth Junction, N.J.)
4-hydroxy-N,N-dimethylbenzamide—ChemBridge Corporation (San Diego, Calif.)
5-chloropyrazine-2-carbonyl chloride—Allichem, LLC (Baltimore, Md.)

### Preparation of Starting Materials and Key Intermediates

### Preparation of 4-bromo-2-fluoro-6-methoxybenzaldehyde (I-1a)

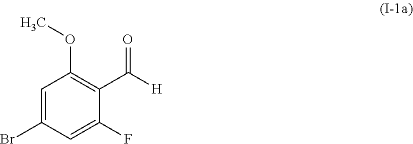

An oven-dried flask was charged with sodium methoxide (6.09 g, 113 mmol) and methanol (100 mL). This mixture was allowed to stir at room temperature under nitrogen for 20 minutes, at which time a nearly homogeneous solution was achieved. A second flask was charged with 4-bromo-2,6-difluorobenzaldehyde (22.65 g, 102.5 mmol) and methanol (100 mL). The sodium methoxide solution was then added dropwise over a 20 minutes period. Following the addition, the reaction was heated to reflux for 8 hours. The reaction was then cooled to room temperature and concentrated to a yellow solid. The crude product was diluted with water and acidified to a pH of 4 with 1N HCl. This solution was extracted with ethyl acetate. The combined organic extracts were dried over magnesium sulfate, filtered, and concentrated. The resulting residue was purified by column chromatography eluting with a gradient of 5-15% ethyl acetate in hexane to afford the title compound 4-bromo-2-fluoro-6-methoxybenzaldehyde (I-1a: 23.88 g, 70%) as a yellow solid.

¹H NMR (400 MHz, CHLOROFORM-d) δ ppm 3.92 (s, 3H), 6.88-6.96 (m, 2H), 10.33 (s, 1H).

### Preparation of 6-bromo-4-methoxy-1H-indazole (I-1b)

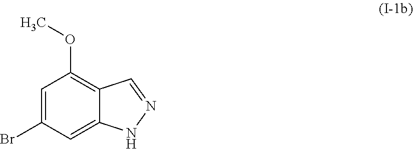

To a stirred suspension of 4-bromo-2-fluoro-6-methoxybenzaldehyde (I-1a: 34.67 g, 148.8 mmol) in ethylene glycol (144 mL) at room temperature was added hydrazine monohydrate (144 mL, 2970 mmol). The resulting suspension was heated to 95° C. under nitrogen for 24 hours. The reaction was then cooled to 5° C. and diluted with water (1 L). The resulting precipitate was filtered off and washed with water. The solid was then dissolved in ethyl acetate, dried over magnesium sulfate, filtered and concentrated to yield the title compound 6-bromo-4-methoxy-1H-indazole (I-1b: 27.97 g, 83%) as a cream colored solid.

¹H NMR (400 MHz, CHLOROFORM-d) δ ppm 3.96 (s, 3H), 6.61 (d, 1 H), 7.26 (br. s, 1H), 8.10 (br. s, 1H).

### Preparation of methyl 4-methoxy-1H-indazole-6-carboxylate (I-1c-1)

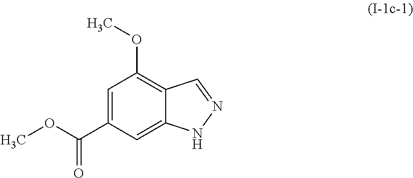

6-bromo-4-methoxy-1H-indazole (I-1b: 6.70 g, 29.5 mmol) was dissolved in methanol (200 mL). To this solution was added 1,3-bis(diphenylphosphino)propane (1460 mg, 3.54 mmol), palladium(II) acetate (662 mg, 2.95 mmol), and triethylamine (8.22 mL, 59.0 mmol). The reaction was pressurized to 50 psi (3.4 atm) of carbon monoxide and was shaken at 60° C. for 18 hours. The reaction was cooled to room temperature and vented. The reaction mixture was then filtered through celite and concentrated. The residue was partitioned between ethyl acetate and water and the layers were separated. The aqueous was extracted again with ethyl acetate. The combined extracts were washed with brine, dried over sodium sulfate, filtered, and concentrated to a yellow solid. Purification by column chromatography eluting with 50-100% ethyl acetate in hexane gave the title compound methyl 4-methoxy-1H-indazole-6-carboxylate (I-1c-1: 4.05 g, 67%) as a yellow solid.

¹H NMR (400 MHz, CHLOROFORM-d) δ ppm 3.94 (s, 3H), 4.01 (s, 3 H), 7.15 (s, 1H), 7.86 (s, 1H), 8.19 (s, 1H).

### Preparation of methyl 4-methoxy-2-methyl-2H-indazole-6-carboxylate (I-1d-1)

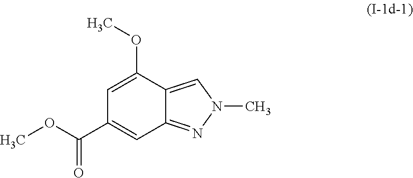

To a stirred solution of methyl 4-methoxy-1H-indazole-6-carboxylate (I-1c-1: 4.0 g, 19.4 mmol) in ethyl acetate (80 mL), was added trimethyloxonium tetrafluoroborate (3.73 g, 25.2 mmol). The reaction was allowed to stir at room temperature for 18 hours. The reaction was then partitioned between ethyl acetate and saturated sodium bicarbonate. The biphasic mixture was filtered through celite, rinsing the filter cake with ethyl acetate. The layers were then separated. The organics were washed with brine, dried over sodium sulfate, filtered and concentrated to a solid. Trituration of the solid with diethyl ether (15 mL) afforded the title compound methyl 4-methoxy-2-methyl-2H-indazole-6-carboxylate (I-1d-1: 4.27 g, 76%) as a cream colored solid.

¹H NMR (400 MHz, CHLOROFORM-d) δ ppm 3.93 (s, 3H), 3.96 (s, 3 H), 4.21 (s, 3H), 6.95 (s, 1H), 7.95 (s, 1H), 8.07 (s, 1H).

### Preparation of methyl 4-hydroxy-2-methyl-2H-indazole-6-carboxylate (I-1e-1)

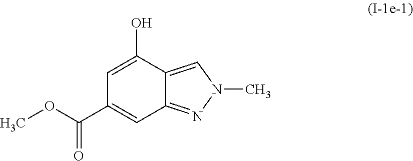

A solution of methyl 4-methoxy-2-methyl-2H-indazole-6-carboxylate (I-1d-1: 3.23 g, 14.6 mmol) in dichloromethane (146 mL) was cooled to 0° C. Boron tribromide (43.9 mL, 43.9 mmol, 1M in dichloromethane) was then added drop-wise over 30 minutes. The ice bath was then removed and the reaction was allowed to warm to room temperature and stir for 18 hours. The reaction was analyzed and found to be incomplete. Additional boron tribromide was added in one portion (22 mL, 22 mmol, 1M in dichloromethane) and the reaction was allowed to stir for another 18 hours. The reaction was then cooled to 0° C. and quenched slowly with methanol. After stirring for 15 minutes, the solution was concentrated to a tan solid. The crude solid was dissolved in methanol (200 mL) and concentrated sulfuric acid (0.10 mL, 1.8 mmol) was added. The mixture was heated to reflux for 10 hours. The reaction was then concentrated to a solid. The solid was taken up in water and neutralized with saturated sodium bicarbonate. This solution was extracted three times with ethyl acetate, dried over magnesium sulfate, filtered, and concentrated. Purification by column chromatography eluting with 30-100% ethyl acetate in heptane gave the title compound methyl 4-hydroxy-2-methyl-2H-indazole-6-carboxylate (I-1e-1: 2.11 g, 70%) as an off-white solid.

¹H NMR (400 MHz, DMSO-d6) δ ppm 3.81 (s, 3H), 4.14 (s, 3H), 6.79 (d, J=1.17 Hz, 1H), 7.70 (s, 1H), 8.35 (s, 1H).

### Preparation of methyl 2-ethyl-4-methoxy-2H-indazole-6-carboxylate (I-1d-2)

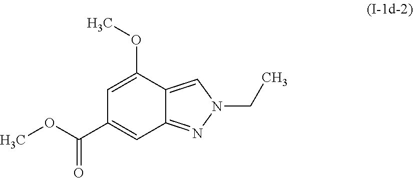

The title compound (I-1d-2) was prepared by a method analogous to that described for I-1d-1, using triethyloxonium tetrafluoroborate.

¹H NMR (400 MHz, CHLOROFORM-d) δ ppm 1.64 (t, J=7.32 Hz, 3 H), 3.94 (s, 3H), 3.97 (s, 3H), 4.48 (q, J=7.42 Hz, 2H), 6.96 (s, 1H), 8.00 (5, 1H), 8.08-8.15 (m, 1H).

### Preparation of methyl 2-ethyl-4-hydroxy-2H-indazole-6-carboxylate (I-1e-2)

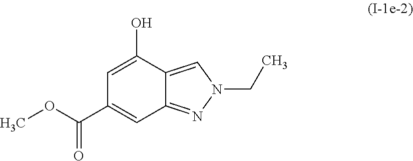

The title compound (I-1e-2) was prepared by a method analogous to that described for I-1e-1, using methyl 2-ethyl-4-methoxy-2H-indazole-6-carboxylate (I-1d-2).

¹HNMR (400 MHz, DMSO-d6) δ ppm 1.47 (t, J=7.6 Hz, 3H), 3.81 (s, 3 H), 4.42 (q, J=7.6 Hz, 2H), 6.79 (s, 1H), 7.71 (s, 1H), 8.40 (s, 1H), 10.29 (5, 1H).

### Preparation of ethyl 4-methoxy-1H-indazole-6-carboxylate (I-1c-2)

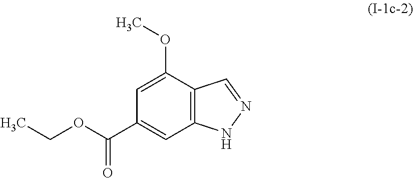

To a stirred suspension of 6-bromo-4-methoxy-1H-indazole (I-1b: 13.99 g, 61.6 mmol) in ethanol (230 mL) and acetonitrile (110 mL) at room temperature in a 1 L autoclave was added triethylamine (44 mL, 315 mmol), 2,2′-bis(diphenylphosphino)-1,1′-binaphthyl (3.84 g, 6.15 mmol), and palladium(II) chloride (2.19 g, 12.35 mmol). The autoclave was then pressurized with carbon monoxide to 20 bar (19.7 atm) and the reaction was stirred at 100° C. After 16 hours, the reaction was cooled to room temperature and vented. The reaction was filtered through celite and concentrated. The resulting residue was taken up in ethyl acetate and stirred for 15 minutes, then filtered. The filtrate was concentrated and then purified by column chromatography eluting with 50-100% ethyl acetate in hexane. The title compound, ethyl 4-methoxy-1H-indazole-6-carboxylate (I-1c-2: 22.6 g, 84%), was obtained as a yellow solid.

¹H NMR (400 MHz, CHLOROFORM-d) δ ppm 1.42 (t, 3H), 4.02 (s, 3 H), 4.42 (q, 2H), 7.16 (m, 1H), 7.87 (m, 1H), 8.18 (s, 1H).

### Preparation of ethyl 4-methoxy-2-methyl-2H-indazole-6-carboxylate (I-1d-3)

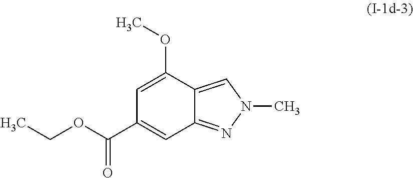

The title compound (I-1d-3) was prepared by a method analogous to that described for Intermediate I-1d-1, using ethyl 4-methoxy-1H-indazole-6-carboxylate (I-1c-2).

¹H NMR (400 MHz, CHLOROFORM-d) δ ppm 1.41 (t, J=7.13 Hz, 3 H), 3.98 (s, 3H), 4.22 (s, 3H), 4.40 (d, J=7.04 Hz, 2H), 6.96 (d, J=0.78 Hz, 1H), 7.96 (s, 1H), 8.09 (s, 1H).

### Preparation of ethyl 4-hydroxy-2-methyl-2H-indazole-6-carboxylate (I-1e-3)

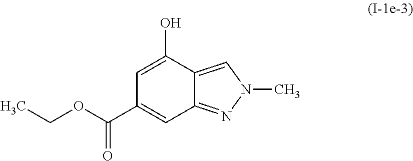

The title compound (I-1e-3) was prepared by a method analogous to that described for Intermediate I-1e-1, using ethanol instead of methanol to form the ester.

¹HNMR (400 MHz, DMSO-d6) δ ppm 1.29 (t, J=7.04 Hz, 3H), 4.14 (s, 3H), 4.26 (d, J=7.23 Hz, 2H), 6.80 (d, J=1.17 Hz, 1H), 7.70 (s, 1H), 8.35 (s, 1H), 10.28 (s, 1H).

### Preparation of ethyl 2-ethyl-4-methoxy-2H-indazole-6-carboxylate (I-1d-4)

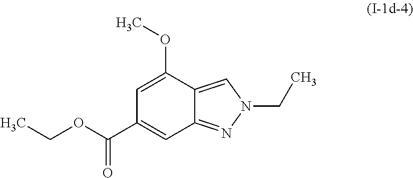

The title compound (I-1d-4) was prepared by a method analogous to that described for Intermediate I-1d-1, using ethyl 4-methoxy-1H-indazole-6-carboxylate (I-1c-2) and triethyloxonium tetrafluoroborate.

¹H NMR (400 MHz, CHLOROFORM-d) δ ppm 1.40 (t, J=7.13 Hz, 3 H), 1.62 (t, J=7.43 Hz, 3H), 3.97 (s, 3H), 4.38 (q, J=7.10 Hz, 2H), 4.45 (m, 2H), 6.95 (d, J=0.78 Hz, 1H), 7.99 (d, J=0.78 Hz, 1H), 8.11 (t, J=0.98 Hz, 1 H).

### Preparation of ethyl 2-ethyl-4-hydroxy-2H-indazole-6-carboxylate (I-1e-4)

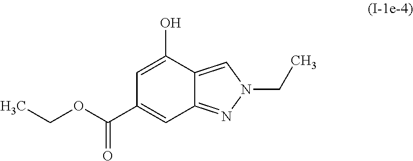

The title compound (I-1e-4) was prepared by a method analogous to that described for I-1e-1, using ethyl 2-ethyl-4-methoxy-2H-indazole-6-carboxylate (I-1d-4) and ethanol instead of methanol to form the ester.

¹HNMR (400 MHz, DMSO-d⁶) δ ppm 1.29 (t, J=7.13 Hz, 3H), 1.47 (t, J=7.33 Hz, 3H), 4.26 (d, J=7.04 Hz, 2H), 4.43 (d, J=7.43 Hz, 2H), 6.80 (d, J=0.98 Hz, 1H), 7.71 (s, 1H), 8.40 (d, J=0.98 Hz, 1H), 10.28 (s, 1H).

### Preparation of ethyl 2-isopropyl-4-methoxy-2H-indazole-6-carboxylate (I-1d-5) and ethyl 4-isopropoxy-2-isopropyl-2H-indazole-6-carboxlate (I-1e-6)

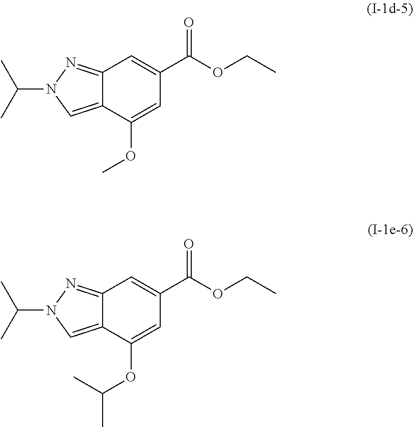

A 2:1 mixture of ethyl 4-methoxy-1H-indazole-6-carboxylate (I-1c-2) and ethyl 4-hydroxy-1H-indazole-6-carboxylate (I-1d-6) (1.57 g) was dissolved in dimethylformamide (20 mL) and a 60% dispersion of sodium hydride in mineral oil (428 mg, 10.7 mmol) was added. After 20 minutes, gas evolution was complete and isopropyl iodide (1.82 g, 10.7 mmol) was added. The reaction was stirred at room temperature for 2 hours. The reaction mixture was then poured into 100 mL of water and extracted with ethyl acetate. The organics were washed twice with water (100 mL) and once with brine (25 mL), dried over magnesium sulfate, filtered and concentrated to an oily solid. Purification by column chromatography (5-30% ethyl acetate in heptane) gave the title compounds:

ethyl 2-isopropyl-4-methoxy-2H-indazole-6-carboxylate (I-1d-5: 300 mg, 16%).

¹H NMR (400 MHz, CHLOROFORM-d) δ ppm 1.42 (t, 3H), 1.65 (d, 6H), 4.00 (s, 3H), 4.40 (m, 2H), 4.80 (m, 1H), 6.96 (m, 1H), 8.04 (d, 1H), 8.16 (m, 1H). MS (M+1): 263.4.

ethyl 4-isopropoxy-2-isopropyl-2H-indazole-6-carboxylate (I-1e-6: 135 mg, 6.5%)

¹H NMR (400 MHz, CHLOROFORM-d) δ ppm 1.45-1.38 (m, 9H), 1.66 (d, 6H), 4.39 (q, 2H), 4.88-4.72 (m, 2H), 6.96 (m, 1H), 8.04 (d, 1H), 8.11 (m, 1H). MS (M+1): 291.4.

### Preparation of ethyl 4-hydroxy-2-isopropyl-2H-indazole-6-carboxylate (I-1e-5)

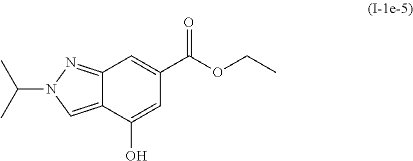

To a solution of ethyl 2-isopropyl-4-methoxy-2H-indazole-6-carboxylate (I-1d-5: 300 mg, 1.14 mmol) in dichloromethane (5.4 mL) at 0° C., was added boron tribromide (0.33 mL, 3.43 mmol). The mixture was allowed to stir and gradually warm to room temperature overnight. The reaction was quenched by the dropwise addition of ethanol and water. The mixture was then concentrated to a yellow solid. This solid was dissolved in ethanol and heated to reflux overnight. The solution was cooled to room temperature and concentrated. The resulting solid was dissolved in water and neutralized with saturated sodium bicarbonate. The solution was extracted with ethyl acetate, dried over magnesium sulfate, filtered, and concentrated. The crude was purified by column chromatography (30-100% ethyl acetate in heptane) to give the title compound ethyl 4-hydroxy-2-isopropyl-2H-indazole-6-carboxylate (I-1e-5: 186 mg, 66%) as a white solid.

¹H NMR (400 MHz, CHLOROFORM-d) δ ppm 1.38 (t, J=7.12 Hz, 3H) 1.66 (d, J=6.83 Hz, 6H) 4.37 (q, J=7.22 Hz, 2H) 4.76-4.88 (m, 1H) 7.06 (d, J=0.98 Hz, 1H) 8.08 (d, J=0.78 Hz, 1H) 8.11 (s, 1H). MS (M+1): 249.3.

### Preparation of ethyl 4-hydroxy-1H-indazole-6-carboxylate (I-1d-6)

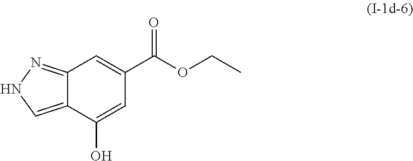

The title compound was prepared by a method analogous to that described for I-1e-1, using ethyl 4-methoxy-1H-indazole-6-carboxylate (I-1c-2) and ethanol instead of methanol to form the ester.

¹HNMR (400 MHz, DMSO-d6) δ ppm 1.32 (t, 3H), 4.28 (q, J=7.09 Hz, 2H), 6.96 (d, J=1.17 Hz, 1H), 7.57 (s, 1H), 8.09 (s, 1H), 10.43 (s, 1H), 13.22 (s, 1H). MS (M+1): 207.3.

### Preparation of (5-chloropyrazin-2-yl)(pyrrolidin-1-yl)methanone (SM-1)

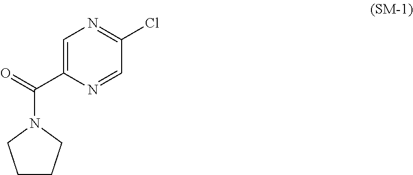

5-chloropyrazine-2-carboxylic acid (1.00 gram, 6.31 mmol) in dichloromethane (30 mL) was treated with catalytic amount of dimethylformamide, followed by (COCl)₂ (0.85 mL, 9.46 mmol). The resulting mixture was stirred over night. The reaction was concentrated and dried under vacuum to give desired 5-chloropyrazine-2-carbonyl chloride as a solid (1.05 g, 100%).

5-chloropyrazine-2-carbonyl chloride (670 mg, 3.79 mmol) was dissolved in dichloromethane (10 mL) and cooled to 0° C. Triethylamine (1.58 mL, 11.4 mmol) and pyrrolidine (0.32 mL, 3.79 mmol) were then added successively drop-wise. Following the addition, the ice bath was removed and the reaction was allowed to warm to room temperature and stir for 1 hour. The reaction was then diluted with dichloromethane and washed with 1N HCl, water, and brine. The organics were dried over sodium sulfate, filtered, and concentrated. Purification by column chromatography eluting with 30-80% ethyl acetate in hexane afforded the desired (5-chloropyrazin-2-yl)(pyrrolidin-1-yl)methanone (SM-1: 677.0 mg, 84.5%).

¹H NMR (400 MHz, CHLOROFORM-d) δ ppm 1.86-1.99 (m, 4H), 3.65-3.71 (m, 2H), 3.73-3.79 (m, 2H), 8.52 (d, J=1.37 Hz, 1H), 8.93 (d, J=1.37 Hz, 1H).

### Preparation of (4-bromo-2-fluorophenyl)(pyrrolidin-1-yl)methanone (SM-2)

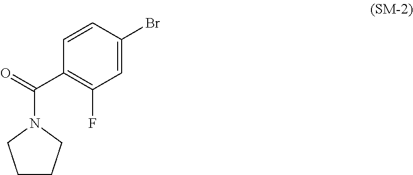

Triethylamine (4.20 mL, 30 mmol), 2-(1H-7-azabenzotriazol-1-yl)-1,1,3,3-tetramethyl uronium hexafluorophosphate methanaminium (4.18 g, 11 mmol), and pyrrolidine (0.92 mL, 11 mmol) were added to a solution of 2-fluoro-4-bromobenzoic acid (2.19 g, 10 mmol) in N,N-dimethylformamide (20 mL). The mixture was stirred at room temperature for 2 hours. The reaction was then quenched with water and extracted with ethyl acetate three times. The combined organics were dried over magnesium sulfate, filtered, and concentrated. The residue was purified by column chromatography (5-55% ethyl acetate in heptanes) to give 3.34 g of a red oil. This oil was taken up in ethyl acetate and washed with 1N HCl and brine. The organic layer was dried over sodium sulfate, filtered, and concentrated to yield (4-bromo-2-fluorophenyl)(pyrrolidin-1-yl)methanone (SM-2: 2.65 g, 97%) as a yellow oil.

¹H NMR (400 MHz, CHLOROFORM-d) δ ppm 1.81-2.02 (m, 4H), 3.28 (t, J=6.64 Hz, 2H), 3.62 (t, J=6.94 Hz, 2H), 7.19-7.42 (m, 3H).

### Preparation of 5-chloro-N,N-dimethylpyrazine-2-carboxamide (SM-3)

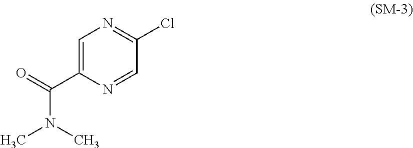

5-chloropyrazine-2-carbonyl chloride (2.13 g, 12.05 mmol) and dimethylamine HCl salt (1.06 g, 12.7 mmol) were suspended in dichloromethane (50 mL) with stirring. Triethylamine (5.04 mL, 36.2 mmol) in dichloromethane (25 mL) was added dropwise at 0° C. to the reaction mixture. The combined solution was warmed up to ambient temperature and stirred for 4 hours. The reaction was diluted with dichloromethane, washed with 1N HCl, water, and brine. The organics were dried over sodium sulfate, filtered, and concentrated. The crude product was purified by column chromatography eluting with a gradient of 30-80% ethyl acetate in heptane to provide desired 5-chloro-N,N-dimethylpyrazine-2-carboxamide (SM-3: 2.24 g, 85%).

¹H NMR (400 MHz, CHLOROFORM-d) δ ppm 3.12 (s, 3H), 3.15 (s, 3 H), 8.53 (d, J=1.37 Hz, 1H), 8.74 (d, J=1.37 Hz, 1H).

### Preparation of 1-(cyclopropylsulfonyl)-4-fluorobenzene (SM-4)

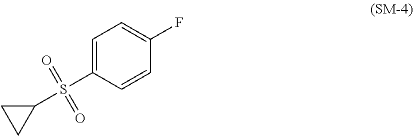

The title compound (SM-4) was prepared according to a previously reported procedure (WO2005121110, page 173).

¹H NMR (400 MHz, CHLOROFORM-d) δ ppm 1.05 (m, 2H), 1.35 (m, 2H), 2.45 (m, 1H), 7.20 (t, 2H), 7.9 (m, 2H).

### Preparation of 3,5-difluoro-N,N-dimethylpicolinamide (SM-5)

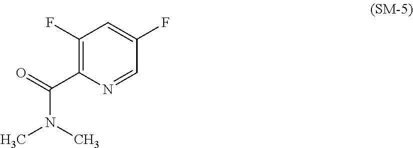

3,5-difluoropyridine-2-carboxylic acid (20.0 g, 125.7 mmol) was dissolved in dichloromethane (190 mL). Thionyl chloride (46 mL, 630 mmol) was added, followed by 5 drops of anhydrous N,N-dimethylformamide. The reaction was heated to reflux and stirred for 18 hours. After cooling to room temperature, the reaction was concentrated and azeotroped with dichloromethane to give the desired 3,5-difluoropicolinoyl chloride (22.3 g, 100%).

3,5-difluoropicolinoyl chloride (11.2 g, 62.9 mmol) was suspended in dichloromethane (60 mL) and cooled to 0° C. Dimethylamine HCl salt (5.13 g, 62.9 mmol) was added. A solution of triethylamine (27.2 mL, 195 mmol) in dichloromethane (20 mL) was then added drop-wise over a period of 3.5 hours. Following the addition, the reaction was allowed to gradually warm to room temperature and stir for 15 hours. The reaction was diluted with saturated sodium bicarbonate and extracted four times with dichloromethane. The combined extracts were dried over magnesium sulfate, filtered, and concentrated. Purification by column chromatography (30-100% ethyl acetate in heptane) gave the title compound 3,5-difluoro-N,N-dimethylpicolinamide (SM-5: 10.5 g, 89%) as an off-white oil.

¹H NMR (400 MHz, CHLOROFORM-d) δ ppm 2.92 (s, 3H), 3.14 (s, 3 H), 7.26-7.30 (m, 1H), 8.34 (s, 1H).

### Preparation of 5-bromo-N,N-dimethylpyrimidine-2-carboxamide (SM-6)

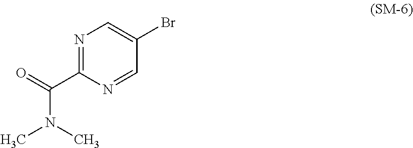

Oxalyl chloride (47.4 g, 369 mmol) was added to a suspension of 5-bromo-pyrimidine-2-carboxylic acid (50 g, 250 mmol) in dichloromethane (821 mL) at room temperature followed by 1-2 drops of N,N-dimethylformamide. The reaction mixture was stirred under nitrogen for 2 hours when LCMS in methanol indicated the presence of the methyl ester and some acid. More N,N-dimethylformamide (0.2 mL) was added to the reaction mixture. The acid dissolved after 30 minutes, at which time LCMS showed the corresponding methyl ester and no starting material peak was observed. The reaction was concentrated and dried in vacuo to afford the crude 5-bromo-pyrimidine-2-carbonyl chloride (55 g, 100%).

The 5-bromo-pyrimidine-2-carbonyl chloride (55 g, 250 mmol) was dissolved in tetrahydrofuran (828 mL) and dimethylamine (2M solution in tetrahydrofuran) (373 mL, 745 mmol) was added portion-wise at room temperature. The reaction was stirred at room temperature under nitrogen for 16 hours, after which time, LCMS indicated completion. The mixture was diluted with ethyl acetate (500 mL) and washed with water (500 mL). The water layer was further extracted with dichloromethane (5×500 mL), all organics combined, and dried over magnesium sulfate. The filtrate was concentrated and then suspended in methyl-t-butylether (650 mL). The solution was then heated to reflux. The hot solution was allowed to cool overnight to afford pink crystals. The crystals were filtered and washed with cold methyl-t-butylether (100 mL). The solid was dried in a vacuum oven at 55° C. for 12 hours to afford the title compound 5-bromo-N,N-dimethylpyrimidine-2-carboxamide (SM-6: 44 g, 77%) as a pink solid.

¹H NMR (400 MHz, CHLOROFORM-d) δ ppm 2.94 (s, 3H) 3.13 (s, 3 H) 8.85 (s, 2H).

### Preparation of azetidin-1-yl(3,5-difluoropyridin-2-yl)methanone (SM-7)

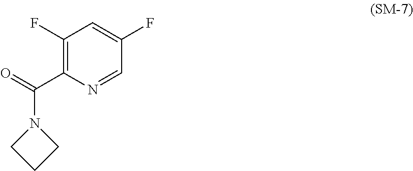

The title compound was prepared by a method analogous to that described for SM-5, using azetidine HCl.

¹H NMR (400 MHz, CHLOROFORM-d) δ ppm 2.28-2.39 (m, 2H), 4.24 (t, J=7.71 Hz, 2H), 4.32 (t, J=7.71 Hz, 2H), 7.26-7.31 (m, 1H), 8.31 (s, 1H). MS (M+1): 199.0.

### Preparation of azetidin-1-yl(5-chloropyrazin-2-yl)methanone (SM-8)

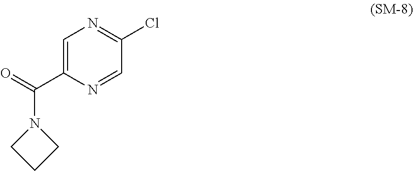

The title compound was prepared by a method analogous to that described for SM-3, using azetidine HCl.

¹H NMR (400 MHz, CHLOROFORM-d) δ ppm 2.33-2.42 (m, 2H), 4.23-4.29 (m, 2H), 4.63-4.69 (m, 2H), 8.51 (d, J=1.37 Hz, 1H), 9.08 (d, J=1.37 Hz, 1H).

### Preparation of N-(5-chloropyrazin-2-yl)-N-methylethanesulfonamide (SM-9)

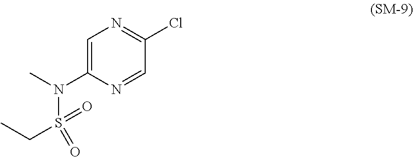

To a solution of N-methyl-ethanesulfonamide (400 mg, 3.25 mmol) in methanol (2.2 mL) was added potassium hydroxide (85%, 214 mg, 3.25 mmol). Solvent was removed and the potassium salt was suspended in dimethylsulfoxide (1.0 mL) and added to a stirred solution of 2,5-dichloropyrazine (484 mg, 3.25 mmol) in dimethylsufoxide (1.0 mL). The resulting dark colored reaction mixture was stirred at room temp for 45 minutes. The reaction mixture was quenched in ice water and the dark suspension was extracted three times with dichloromethane. The combined organics were washed with brine, dried over sodium sulfate and flash chromatographed eluting with a 0-40% gradient of ethyl acetate in heptane to afford N-(5-chloropyrazin-2-yl)-N-methyl ethanesulfonamide (SM-9: 195 mg, 25.5%).

¹H NMR (400 MHz, CHLOROFORM-d) ppm 1.32 (t, J=7.43 Hz, 3 H), 3.26 (q, J=7.30 Hz, 2H), 3.42 (s, 3H), 8.32 (d, J=1.37 Hz, 1H), 8.56 (d, J=1.17 Hz, 1H). MS (M+1) 236.0.

### Preparation of N-(5-bromopyrazin-2-yl)acetamide (SM-10)

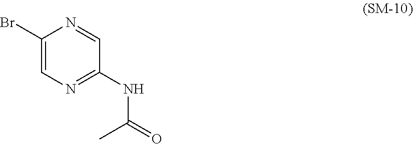

To a solution of 5-bromopyrazin-2-amine (413 mg, 2.37 mmol) in dichloromethane (11.9 mL) was added pyridine (5.82 mL, 71.2 mmol) and acetyl chloride (0.27 mL, 3.80 mmol). The reaction stirred for 1 hour and was then concentrated. Purification by column chromatography (0-20% ethyl acetate in heptane) afforded the desired product N-(5-bromopyrazin-2-yl)acetamide (SM-10: 510 mg, 99%) as a white solid.

¹H NMR (400 MHz, CHLOROFORM-d) δ ppm 2.27 (s, 3H), 8.09 (br.s., 1H), 8.32 (d, J=1.56 Hz, 1H), 9.34 (s, 1H). MS (M+1): 216.1.

### Preparation of N-(5-bromopyrazin-2-yl)-N-methylacetamide (SM-11)

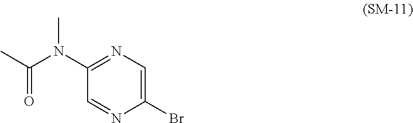

To a solution of N-(5-bromopyrazin-2-yl)acetamide (SM-10: 250 mg, 1.16 mmol) in dimethylformamide (11.6 mL) was added a 60% dispersion of sodium hydride in mineral oil (92.6 mg, 2.31 mmol). The reaction was stirred at room temperature for 15 minutes and then methyl iodide (0.288 mL, 4.63 mmol) was added. After 16 hours, the reaction was diluted with ethyl acetate, washed with water and brine, dried over sodium sulfate, filtered, and concentrated. Purification by column chromatography (20-80% ethyl acetate in heptane) gave N-(5-bromopyrazin-2-yl)-N-methylacetamide (SM-11: 68.5 mg, 26%) as a white solid.

¹H NMR (400 MHz, CHLOROFORM-d) δ ppm 2.30 (s, 3H), 3.46 (s, 3 H), 8.47-8.51 (m, 1H), 8.81 (br. s., 1H). MS (M+1): 232.2.

### Preparation of (E)-N′-(5-bromopyrazin-2-yl)-N,N-dimethylformamidine (SM-12)

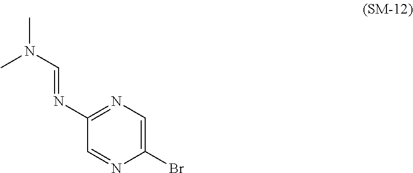

5-bromopyrazin-2-amine (250 mg, 1.44 mmol) was dissolved in N,N-dimethylformamide dimethyl acetal (1.92 mL, 14.4 mmol) and heated to 60° C. for 2 hours. The reaction was concentrated to afford (E)-N′-(5-bromopyrazin-2-yl)-N,N-dimethylformamidine (SM-12: 329 mg, 100%) as a yellow solid.

¹H NMR (400 MHz, CHLOROFORM-d) δ ppm 3.10 (d, J=6.25 Hz, 6 H) 8.03 (d, J=1.37 Hz, 1H) 8.18 (d, J=1.37 Hz, 1H) 8.42 (s, 1H). MS (M+1): 230.3.

### Preparation of 5-chloro-N,N-bis(2,4-dimethoxybenzyl)pyrazine-2-carboxamide (SM-13)

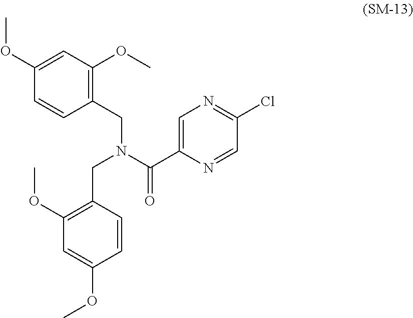

To a stirred solution of 5-chloropyrazine-2-carbonyl chloride (100 mg, 0.565 mmol) in tetrahydrofuran (2.82 mL) at 0° C., was added bis(2,4-dimethoxybenzyl)amine (215 mg, 0.678 mmol) and triethylamine (0.118 mL, 0.847 mmol). The reaction was allowed to warm to room temperature and stir for 2 hours. The reaction was diluted with 5 mL ethyl acetate, washed with saturated sodium bicarbonate (2×1 mL) and water (1 mL), dried over sodium sulfate, filtered, and concentrated. Column chromatography (10-50% ethyl acetate in heptane) gave the title compound 5-chloro-N,N-bis(2,4-dimethoxybenzyl)pyrazine-2-carboxamide (SM-13: 226 mg, 87%).

¹H NMR (400 MHz, CHLOROFORM-d) δ ppm 3.58 (s, 3H) 3.77 (d, J=1.76 Hz, 6H) 3.79 (s, 3H) 4.53 (s, 2H) 4.61 (s, 2H) 6.34 (d, J=2.35 Hz, 1 H) 6.39 (dd, J=8.31, 2.25 Hz, 1H) 6.42-6.50 (m, 2H) 6.97 (d, J=8.40 Hz, 1 H) 7.27 (d, J=8.40 Hz, 1H) 8.49 (d, J=1.37 Hz, 1H) 8.68 (d, J=1.37 Hz, 1 H). MS (M+1): 458.1.

### Preparation of N,N-bis(2,4-dimethoxybenzyl)-4-iodobenzenesulfonamide (SM-14)

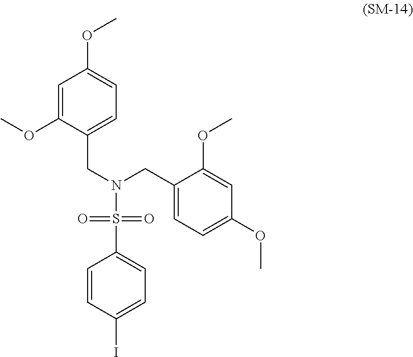

To a solution of 4-iodo-benzenesulfonyl chloride (3.02 g, 9.98 mmol) in tetrahydrofuran (50 mL) at 0° C. was added triethylamine (1.94 g, 15 mmol) followed by bis(2,4-dimethoxybenzyl)amine (3.49 g, 11.0 mmol). The solution was allowed to slowly warm to room temperature and then stirred at room temperature overnight. The resulting suspension was diluted with ethyl acetate (100 mL). The organic phase was washed with 100 mL each 0.1 M HCl and water, 50 mL saturated sodium bicarbonate solution and 25 mL brine. The organic layer was dried over magnesium sulfate, filtered and concentrated to afford the title compound N,N-bis(2,4-dimethoxybenzyl)-4-iodobenzenesulfonamide (SM-14).

¹H NMR (400 MHz, CHLOROFORM-d) ppm 3.61 (s, 6H), 3.79 (s, 6 H), 4.38 (s, 4H), 6.27 (d, 2H), 6.39 (dd, 2H), 7.15 (d, 2H), 7.38-7.32 (m, 2 H), 7.75-7.69 (m, 2H).

### Preparation of N,N-bis(2,4-dimethoxybenzyl)-4-fluorobenzenesulfonamide (SM-15)

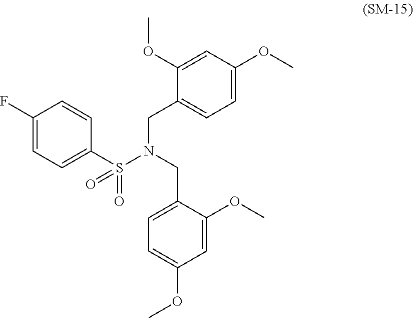

To a solution of 4-fluorobenzene-1-sulfonyl chloride (1.00 g, 5.14 mmol) in dry dichloromethane (30.0 mL) was added bis(2,4-dimethoxybenzyl)amine (2.45 g, 7.71 mmol) and triethylamine (1.56 g, 15.4 mmol). The reaction was stirred at room temperature overnight. The reaction mixture was washed with 1N HCl (2×20 mL). The organic layer was dried and concentrated under reduced pressure. Column chromatography (15-25% ethyl acetate in heptane) gave the title compound N,N-bis(2,4-dimethoxybenzyl)-4-fluorobenzenesulfonamide (SM-15: 2.12 g, 86.8%) as a white solid.

¹H NMR (400 MHz, CHLOROFORM-d) δ ppm 3.61 (s, 6H), 3.77 (s, 6 H), 4.38 (s, 4H), 6.27 (s, 2H), 6.37-6.39 (m, 2H), 7.02-7.06 (m, 2H), 7.13-7.15 (d, 2H), 7.63-7.67 (m, 2H).

### Preparation of methyl 4-(5-(dimethylcarbamoyl)pyrazin-2-yloxy)-2-methyl-2H-indazole-6-carboxylate (I-1f-1)

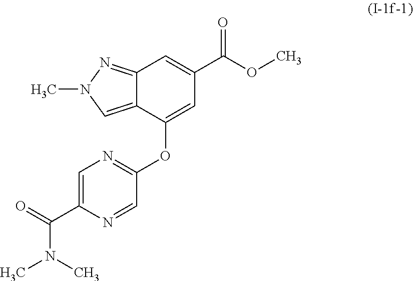

To a solution of methyl 4-hydroxy-2-methyl-2H-indazole-6-carboxylate (I-1e-1: 1.65 g, 8.01 mmol), cesium carbonate (2.86 g, 8.77 mmol), and copper iodide (390 mg, 2.05 mmol) in N,N-dimethylformamide (23 mL) was added 5-chloro-N,N-dimethylpyrazine-2-carboxamide (SM-3: 1.09 g, 5.85 mmol). The reaction was heated to 110° C. and stirred for 2 hours. The reaction was then cooled to room temperature and concentrated. The crude material was taken up in ethyl acetate (200 mL) and washed 2 times with water (2×25 mL) and once with brine (25 mL). The organic layer was dried over sodium sulfate, filtered, and concentrated to remove most of the solvent. To this mixture was added a small amount of heptane (2 mL), which caused the formation of a solid. The solid was collected by filtration and dried under vacuum to give methyl 4-(5-(dimethylcarbamoyl)pyrazin-2-yloxy)-2-methyl-2H-indazole-6-carboxylate (I-1f-1: 1.46 g, 70%) as a yellow-brown solid.

¹H NMR (400 MHz, CHLOROFORM-d) δ ppm 3.07-3.24 (m, 6H), 3.93 (s, 3H), 4.22 (s, 3H), 7.50 (s, 1H), 7.72 (s, 1H), 8.33-8.45 (m, 2H), 8.48 (s, 1H).

### Preparation of methyl 2-methyl-4-(5-(pyrrolidine-1-carbonyl)pyrazin-2-yloxy)-2H-indazole-6-carboxylate (I-1f-2)

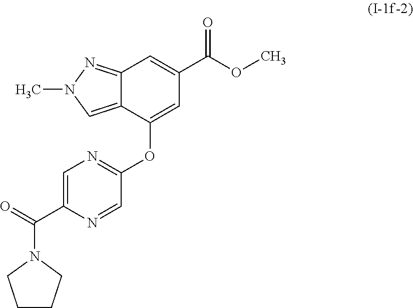

The title compound was prepared by a method analogous to that described for I-1f-1, using (5-chloropyrazin-2-yl)(pyrrolidin-1-yl)methanone (SM-1).

¹H NMR (400 MHz, CHLOROFORM-d) δ ppm 1.83-1.98 (m, 4H), 3.65 (m, 2H), 3.77 (m, 2H), 3.90 (s, 3H), 4.19 (s, 3H), 7.47 (d, J=0.78 Hz, 1H), 7.70 (s, 1H), 7.97 (s, 2H), 8.34-8.40 (m, 1H).

### Preparation of methyl 4-(5-(dimethylcarbamoyl)pyrazin-2-yloxy)-2-ethyl-2H-indazole-6-carboxylate (I-1f-3)

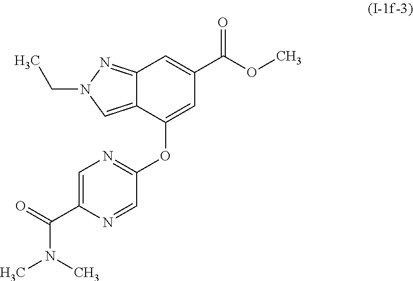

The title compound was prepared by a method analogous to that described for I-1f-1, using methyl 2-ethyl-4-hydroxy-2H-indazole-6-carboxylate (I-1e-2).

¹H NMR (400 MHz, CHLOROFORM-d) δ ppm 1.63 (t, J=7.6 Hz, 3H), 3.14 (s, 3H), 3.16 (s, 3H), 3.93 (s, 3H), 4.47 (q, J=7.6 Hz, 2H), 7.49 (s, 1 H), 7.76 (s, 1H), 8.42 (s, 2H), 8.49 (s, 1H).

### Preparation of methyl 4-(3-fluoro-4-(pyrrolidine-1-carbonyl)phenoxy)-2-methyl-2H-indazole-6-carboxylate (I-1f-4)

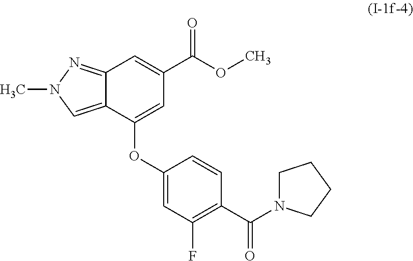

To a solution of methyl 4-hydroxy-2-methyl-2H-indazole-6-carboxylate (I-1e-1: 155 mg, 0.752 mmol) in toluene (2.5 mL), was added (4-bromo-2-fluorophenyl)(pyrrolidin-1-yl)methanone (SM-2: 205 mg, 0.752 mmol) and potassium phosphate (319 mg, 1.50 mmol). The reaction mixture was degassed with nitrogen for approximately 10 minutes. Then added palladium(II) acetate (13.5 mg, 0.06 mmol) and 2-di-t-butylphosphino-2′,4′,6′-tri-isopropyl-1,1′-biphenyl (25.5 mg, 0.06 mmol). Heated reaction to reflux. After 48 hours, LCMS showed unreacted starting material remained. Added additional palladium(II) acetate (13.5 mg, 0.06 mmol) and 2-di-t-butylphosphino-2′,4′,6′-tri-isopropyl-1,1′-biphenyl (25.5 mg, 0.06 mmol) in toluene (1 mL), and continued to heat at reflux for another 18 hours. Cooled reaction to room temperature and concentrated. Purification by column chromatography eluting with 30-100% ethyl acetate in heptane, then 0-9% methanol in ethyl acetate gave the desired methyl 4-(3-fluoro-4-(pyrrolidine-1-carbonyl)phenoxy)-2-methyl-2H-indazole-6-carboxylate (I-1f-4: 40 mg, 13%) as a yellow solid.

¹H NMR (400 MHz, CHLOROFORM-d) δ ppm 1.80-1.98 (m, 4H), 3.32 (t, J=6.55 Hz, 2H), 3.55-3.63 (m, 2H), 3.89 (s, 3H), 4.18 (s, 3H), 6.72 (dd, J=10.75, 2.35 Hz, 1H), 6.83 (dd, J=8.50, 2.25 Hz, 1H), 7.23 (d, J=0.98 Hz, 1H), 7.36 (t, J=8.11 Hz, 1H), 7.74 (s, 1H), 8.27 (s, 1H).

### Preparation of methyl 2-methyl-4-(5-(trifluoromethyl)pyrazin-2-yloxy)-2H-indazole-6-carboxylate (I-1f-5)

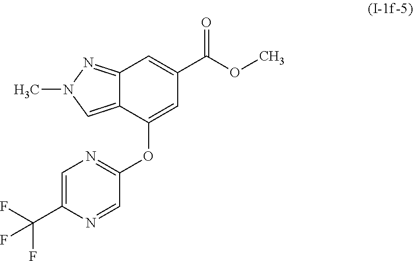

To a solution of methyl 4-hydroxy-2-methyl-2H-indazole-6-carboxylate (I-1e-1: 150 mg, 0.72 mmol) and cesium carbonate (261 mg, 0.8 mmol) in N,N-dimethylformamide (1.5 mL) was added 2-chloro-5-(trifluoromethyl)pyrazine (265 mg, 1.45 mmol). The reaction was heated to 100° C. for 0.5 hour, then cooled to room temperature and concentrated. The crude reaction mixture was partitioned between ethyl acetate and water. The layers were separated, the organics washed with brine, dried over sodium sulfate, filtered, and concentrated. Purification by column chromatography (30-75% ethyl acetate in heptane) afforded the title compound methyl 2-methyl-4-(5-(trifluoromethyl)pyrazin-2-yloxy)-2H-indazole-6-carboxylate (I-1f-5: 220 mg, 86%) as a yellow gum.

¹H NMR (400 MHz, CHLOROFORM-d) δ ppm 3.93 (s, 3H), 4.22 (s, 3 H), 7.52 (d, J=1.17 Hz, 1H), 7.75 (s, 1H), 8.41 (s, 1H), 8.42 (t, J=1.07 Hz, 1 H), 8.57 (d, J=0.98 Hz, 1H).

### Preparation of methyl 2-methyl-4-(4-(methylsulfonyl)phenoxy)-2H-indazole-6-carboxylate (I-1f-6)

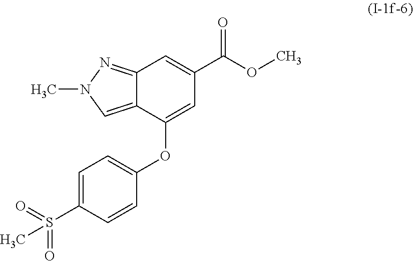

The title compound was prepared by a method analogous to that described for I-1f-5, using 1-fluoro-4-(methylsulfonyl)benzene.

¹H NMR (400 MHz, CHLOROFORM-d) δ ppm 3.07 (s, 3H), 3.93 (s, 3 H), 4.22 (s, 3H), 7.14 (s, 1H), 7.17 (s, 1H), 7.30 (d, J=1.17 Hz, 1H), 7.76 (s, 1H), 7.89 (s, 1H), 7.92 (s, 1H), 8.32-8.36 (m, 1H).

### Preparation of methyl 2-ethyl-4-(4-(methylsulfonyl)phenoxy)-2H-indazole-6-carboxylate (I-1f-7)

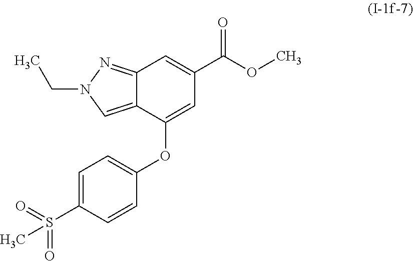

The title compound was prepared by a method analogous to that described for I-1f-5, using methyl 2-ethyl-4-hydroxy-2H-indazole-6-carboxylate (I-1e-2) and 1-fluoro-4-(methylsulfonyl)benzene.

¹H NMR (400 MHz, CHLOROFORM-d) δ ppm 1.63 (t, J=7.2 Hz, 3H), 3.07 (s, 3H), 3.93 (s, 3H), 4.47 (q, J=7.2 Hz, 2H), 7.15-7.17 (d, J=9.2 Hz, 2H), 7.28 (s, 1H), 7.81 (s, 1H), 7.92-7.89 (d, J=9.2 Hz, 2H), 8.36 (s, 1H).

### Preparation of ethyl 4-(4-(ethylsulfonyl)phenoxy)-2-methyl-2H-indazole-6-carboxylate (I-1f-8)

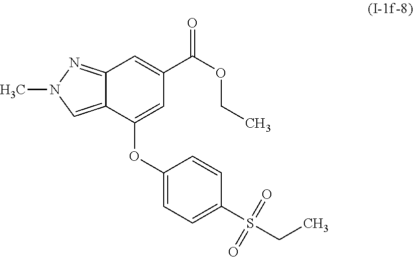

The title compound was prepared by a method analogous to that described for I-1f-5, using ethyl 4-hydroxy-2-methyl-2H-indazole-6-carboxylate (I-1e-3) and 1-fluoro-4-(ethylsulfonyl)benzene.

¹H NMR (400 MHz, CHLOROFORM-d) δ ppm 1.24 (m, 3H), 1.35 (m, 3H), 3.07 (q, J=7.30 Hz, 2H), 4.17 (s, 3H), 4.34 (m, 2H), 7.10 (d, J=7.62 Hz, 2H), 7.27 (s, 1H), 7.71 (s, 1H), 7.80 (d, J=7.82 Hz, 2H), 8.30 (s, 1H).

### Preparation of ethyl 4-(4-(cyclopropylsulfonyl)phenoxy)-2-methyl-2H-indazole-6-carboxylate (I-1f-9)

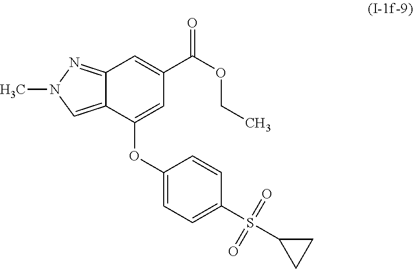

The title compound was prepared by a method analogous to that described for I-1f-5, using ethyl 4-hydroxy-2-methyl-2H-indazole-6-carboxylate (I-1e-3) and 1-(cyclopropylsulfonyl)-4-fluorobenzene (SM-4).

¹H NMR (400 MHz, CHLOROFORM-d) δ ppm 1.04 (dd, J=7.82, 1.95 Hz, 2H), 1.34 (dd, J=4.69, 1.95 Hz, 2H), 1.39 (t, J=7.13 Hz, 3H), 2.41-2.53 (m, 1H), 4.22 (s, 3H), 4.39 (q, J=7.23 Hz, 2H), 7.11-7.14 (m, 1H), 7.14-7.16 (m, 1H), 7.32 (d, J=1.17 Hz, 1H), 7.74 (s, 1H), 7.82-7.85 (m, 1H), 7.85-7.88 (m, 1H), 8.32-8.38 (m, 1H).

### Preparation of ethyl 4-(4-(cyclopropylsulfonyl)phenoxy)-2-ethyl-2H-indazole-6-carbon/late (I-1f-10)

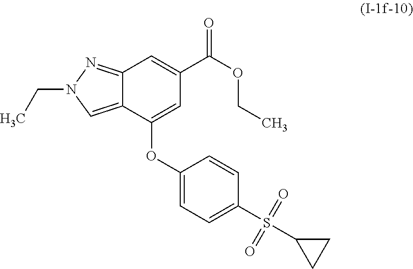

The title compound was prepared by a method analogous to that described for (I-1f-5), using ethyl 2-ethyl-4-hydroxy-2H-indazole-6-carboxylate (I-1e-4) and 1-(cyclopropylsulfonyl)-4-fluorobenzene (SM-4).

MS (M+1): 414.8.

### Preparation of methyl 4-(2-(dimethylcarbamoyl)pyrimidin-5-yloxy)-2-methyl-2H-indazole-6-carboxylate (I-1f-11)

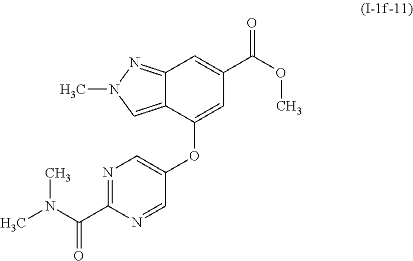

The title compound was prepared by a method analogous to that described for (I-1f-5), using 5-bromo-N,N-dimethylpyrimidine-2-carboxamide (SM-6).

¹H NMR (400 MHz, CHLOROFORM-d) δ ppm 2.99 (s, 3H), 3.15 (s, 3 H), 3.92 (s, 3H), 4.24 (s, 3H), 7.26 (s, 1H), 7.84 (s, 1H), 8.36 (s, 1H), 8.56 (br. s, 2H).

### Preparation of methyl 4-(6-(dimethylcarbamoyl)-5-fluoropyridin-3-yloxy)-2-methyl-2H-indazole-6-carboxylate (I-1f-12)

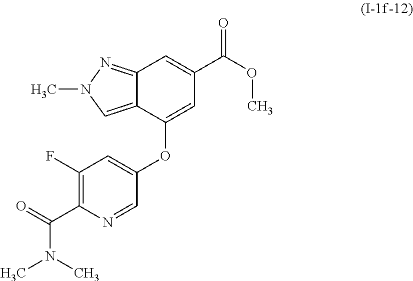

The title compound was prepared by a method analogous to that described for (I-1f-5), using 3,5-difluoro-N,N-dimethylpicolinamide (SM-5).

¹H NMR (400 MHz, CHLOROFORM-d) δ ppm 2.98 (s, 3H), 3.15 (s, 3 H), 3.94 (s, 3H), 4.25 (s, 3H), 7.09 (dd, J=9.86, 2.25 Hz, 1H), 7.32 (s, 1H), 7.81 (s, 1H), 8.30 (s, 1H), 8.37 (s, 1H).

### Preparation of methyl 2-ethyl-4-(4-(ethylsulfonyl)phenoxy)-2H-indazole-6-carbon/late (I-1f-13)

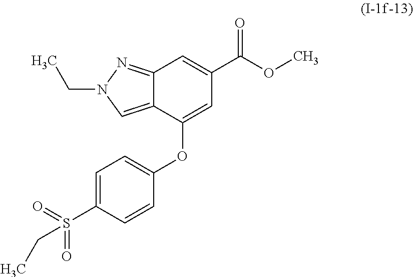

The title compound was prepared by a method analogous to that described for (I-1f-5), using methyl 2-ethyl-4-hydroxy-2H-indazole-6-carboxylate (I-1e-2) and 1-fluoro-4-(ethylsulfonyl)benzene.

¹H NMR (400 MHz, CHLOROFORM-d) δ ppm 1.30 (q, J=7.6 Hz, 3H), 1.63 (t, J=7.6 Hz, 3H), 3.12 (q, J=7.6 Hz, 2H), 3.93 (s, 3H), 4.47 (q, J=7.6 Hz, 2H), 7.15-7.17 (d, J=8.8 Hz, 2H), 7.30 (s, 1H), 7.79 (s, 1H), 7.85-7.87 (d, J=8.8 Hz, 2H), 8.36 (s, 1H).

### Preparation of methyl 4-(6-(dimethylcarbamoyl)-5-fluoropyridin-3-yloxy)-2-ethyl-2H-indazole-6-carboxylate (I-1f-14)

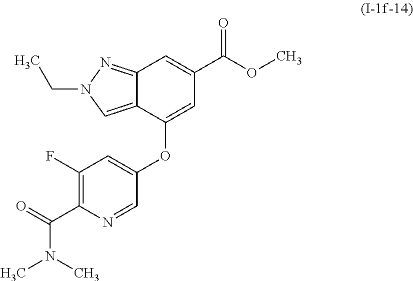

The title compound was prepared by a method analogous to that described for (I-1f-5), using methyl 2-ethyl-4-hydroxy-2H-indazole-6-carboxylate (I-1e-2) and 3,5-difluoro-N,N-dimethylpicolinamide (SM-5).

¹H NMR (400 MHz, CHLOROFORM-d) δ ppm 1.63 (t, J=7.2 Hz, 3H), 2.97 (s, 3H), 3.14 (s, 3H), 3.92 (s, 3H), 4.48 (q, J=7.2 Hz, 2H), 7.06-7.10 (m, 1H), 7.29 (s, 1H), 7.84 (s, 1H), 8.29-8.30 (m, 1H), 8.37 (s, 1H).

### Preparation of methyl 2-methyl-4-(5-(N-methylacetamido)pyrazin-2-yloxy)-2H-indazole-6-carboxylate (I-1f-15)

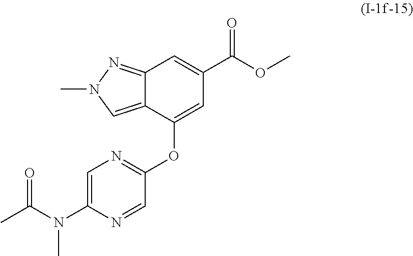

Methyl 4-hydroxy-2-methyl-2H-indazole-6-carboxylate (I-1e-1: 55.7 mg, 0.27 mmol) was dissolved in dimethylformamide (0.68 mL). Added N-(5-bromopyrazin-2-yl)-N-methylacetamide (SM-11: 68.1 mg, 0.296 mmol), potassium carbonate (112 mg, 0.808 mmol), and copper chloride (0.800 mg, 0.0080 mmol). Purged reaction flask with nitrogen and then added 2,2,6,6-tetramethylheptane-3,5-dione (6.00 L, 0.027 mmol). The reaction was heated in a sealed tube at 120° C. for 18 hours. The reaction mixture was then filtered and concentrated. The crude was absorbed onto silica gel and purified using column chromatography (0-5% methanol in ethyl acetate) to give the title compound methyl 2-methyl-4-(5-(N-methylacetamido)pyrazin-2-yloxy)-2H-indazole-6-carboxylate (I-1f-15: 45 mg, 47%) as an orange oil.

¹H NMR (400 MHz, CHLOROFORM-d) δ ppm 2.88 (s, 3H), 2.95 (s, 3 H), 3.94 (s, 3H), 4.24 (s, 3H), 7.47 (s, 1H), 7.83 (s, 1H), 8.01 (s, 1H), 8.33 (s, 1H), 8.37-8.41 (m, 1H). MS (M+1): 356.4.

### Preparation of (E)-ethyl 4-(5-((dimethylamino)methyleneamino)pyrazin-2-yloxy)-2-ethyl-2H-indazole-6-carboxylate (I-1f-16)

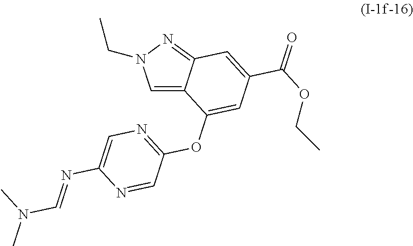

Ethyl 2-ethyl-4-hydroxy-2H-indazole-6-carboxylate (I-1e-4: 50 mg, 0.22 mmol) was dissolved in dimethylformamide (0.54 mL). Added (E)-N′-(5-bromopyrazin-2-yl)-N,N-dimethylformamidine (SM-12: 54.3 mg, 0.237 mmol), potassium carbonate (89.2 mg, 0.037 mmol), and copper chloride (2.20 mg, 0.022 mmol). Purged reaction flask with nitrogen and then added 2,2,6,6-tetramethylheptane-3,5-dione (9.00 uL, 0.043 mmol). The reaction was heated in a sealed tube at 120° C. for 18 hours. The reaction was then cooled to room temperature, diluted with ethyl acetate, and washed with water and brine. The organics were dried over sodium sulfate, filtered, and concentrated. Purification by column chromatography (0-10% methanol in ethyl acetate) gave the desired (E)-ethyl 4-(5-((dimethylamino)methyleneamino)pyrazin-2-yloxy)-2-ethyl-2H-indazole-6-carboxylate (I-1f-16: 50 mg, 61%) as an orange gum.

¹H NMR (400 MHz, CHLOROFORM-d) δ ppm 1.36 (t, J=7.13 Hz, 3 H), 1.60 (t, J=7.43 Hz, 3H), 3.10 (d, J=5.28 Hz, 6H), 4.35 (q, J=7.23 Hz, 2 H), 4.44 (q, J=7.23 Hz, 2H), 7.27 (s, 1H), 7.79 (s, 1H), 7.91 (d, J=1.37 Hz, 1H), 8.07 (d, J=1.17 Hz, 1H), 8.30 (s, 1H), 8.38 (s, 1H). MS (M+1): 383.5.

### Preparation of ethyl 4-(5-aminopyrazin-2-yloxy)-2-ethyl-2H-indazole-6-carboxylate (I-1g-1)

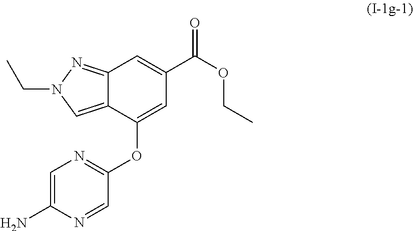

(E)-ethyl 4-(5-((dimethylamino)methyleneamino)pyrazin-2-yloxy)-2-ethyl-2H-indazole-6-carboxylate (I-1f-16: 580 mg, 1.52 mmol) was dissolved in ethanol (15.2 mL). Added 30% ammonium hydroxide (10.0 mL) and heated reaction to 60° C. for 40 hours. The reaction was then concentrated and the crude was adsorbed onto silica gel and purified by column chromatography (0-5% methanol in ethyl acetate) to give the desired ethyl 4-(5-aminopyrazin-2-yloxy)-2-ethyl-2H-indazole-6-carboxylate (I-1g-1: 247 mg, 50%) as a yellow solid.

¹H NMR (400 MHz, CHLOROFORM-d) δ ppm 1.36 (t, J=7.13 Hz, 3 H), 1.62 (t, J=7.33 Hz, 3H), 4.35 (q, J=7.04 Hz, 2H), 4.40-4.51 (m, 4H), 7.16 (s, 1H), 7.65 (d, J=1.37 Hz, 1H), 7.87 (s, 1H), 7.96 (d, J=1.37 Hz, 1 H), 8.28 (s, 1H). MS (M+1): 328.4.

### Preparation of ethyl 4-(5-acetamidopyrazin-2-yloxy)-2-ethyl-2H-indazole-6-carboxylate (I-1h-1)

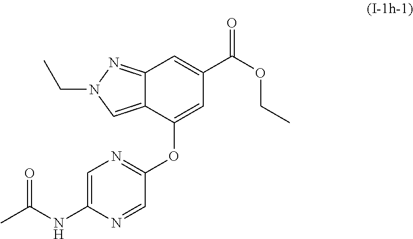

Ethyl 4-(5-aminopyrazin-2-yloxy)-2-ethyl-2H-indazole-6-carboxylate (I-1g-1: 55 mg, 0.17 mmol) was dissolved in dichloromethane (1.68 mL) and pyridine (0.40 mL, 4.00 mmol). Added acetyl chloride (14 uL, 0.202 mmol) and let stir for 2 hours. The reaction was then concentrated, adsorbed onto silica gel and purified by column chromatography (80-100% ethyl acetate in heptane) to afford ethyl 4-(5-acetamidopyrazin-2-yloxy)-2-ethyl-2H-indazole-6-carboxylate (I-1h-1: 57 mg, 92%) as a white solid.

¹H NMR (400 MHz, CHLOROFORM-d) δ ppm 1.38 (t, J=7.13 Hz, 3 H), 1.62 (t, J=7.33 Hz, 3H), 2.23 (s, 3H), 4.37 (q, J=7.10 Hz, 2H), 4.46 (q, J=7.43 Hz, 2H), 7.37 (d, J=0.98 Hz, 1H), 7.75 (br. s., 1H), 7.79 (d, J=0.78 Hz, 1H), 8.12 (d, J=1.37 Hz, 1H), 8.37 (t, J=0.98 Hz, 1H), 9.07 (s, 1H). MS (M+1): 370.5.

### Preparation of ethyl 2-ethyl-4-({5-[(methylsulfonyl)amino]pyrazin-2-yl}oxy)-2H-indazole-6-carboxylate (I-1h-2)

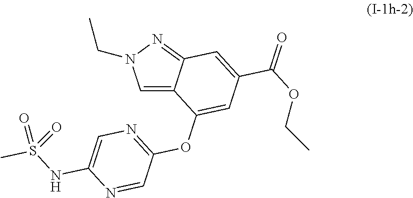

To a solution of ethyl 4-[(5-aminopyrazin-2-yl)oxy]-2-ethyl-2H-indazole-6-carboxylate (I-1g-1: 80 mg, 0.24 mmol) in dichloromethane (2.44 mL) containing pyridine (0.5 mL, 30 mmol) at room temperature was added methanesulfonyl chloride (0.047 mL, 0.610 mmol) in one portion. The reaction was stirred at room temperature overnight under nitrogen. The suspension was solubilized with methanol. Silica gel was added and solvents were concentrated. The material was flash chromatographed eluting with a 80-100% gradient of ethyl acetate in heptane to afford the desired ethyl 2-ethyl-4-({5-[(methylsulfonyl)amino]pyrazin-2-yl}oxy)-2H-indazole-6-carboxylate (I-1h-2: 51 mg, 52%).

¹H NMR (400 MHz, CHLOROFORM-d) ppm 1.39 (t, J=7.04 Hz, 3 H), 1.63 (t, J=7.33 Hz, 3H), 3.26 (s, 3H), 4.38 (q, J=7.04 Hz, 2H), 4.48 (q, J=7.23 Hz, 2H), 7.39 (d, J=0.98 Hz, 1H), 7.83 (s, 1H), 8.09 (d, J=1.37 Hz, 1H), 8.22 (d, J=1.37 Hz, 1H), 8.38-8.41 (m, 1H). MS (M+1): 406.4

### Preparation of ethyl 2-ethyl-4-({5-[methyl(methylsulfonyl)amino]pyrazin-2-yl}oxy)-2H-indazole-6-carboxylate (I-1i-1)

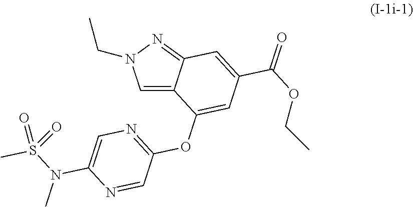

To a solution of ethyl 2-ethyl-4-({5-[(methylsulfonyl)amino]pyrazin-2-yl}oxy)-2H-indazole-6-carboxylate (I-1h-2: 50 mg, 0.12 mmol) in N,N-dimethylformamide (1.23 mL) was added sodium hydride (60% dispersion in mineral oil, 8.8 mg, 0.22 mmol). After 30 minutes a single portion of methyl iodide (0.038 mL, 0.615 mmol) was added and the mixture was stirred at room temperature overnight. The mixture was diluted with ethyl acetate, washed three times with water, once with brine, dried over sodium sulfate, filtered, concentrated, and flash chromatographed eluting with a 70-100% gradient of ethyl acetate in heptane to afford the title compound ethyl 2-ethyl-4-({5-[methyl(methylsulfonyl)amino]pyrazin-2-yl}oxy)-2H-indazole-6-carboxylate (I-1i-1: 52 mg, 84%).

¹H NMR (400 MHz, CHLOROFORM-d) ppm 1.41 (m, 3H) 1.65 (t, J=7.33 Hz, 3H) 3.03 (s, 3H) 3.39 (s, 3H) 4.41 (q, J=7.10 Hz, 2H) 4.50 (q, J=7.43 Hz, 2H) 7.43-7.49 (m, 1H) 7.84-7.88 (m, 1H) 8.25-8.28 (m, 1H) 8.27-8.30 (m, 1H) 8.40-8.46 (m, 1H).

### Preparation of methyl 4-(6-(azetidine-1-carbonyl)-5-fluoropyridin-3-yloxy)-2-ethyl-2H-indazole-6-carboxylate (I-1f-17)

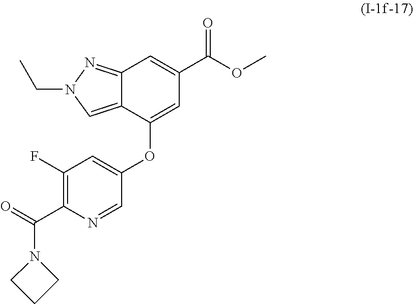

The title compound was prepared by a method analogous to that described for I-1f-5, using methyl 2-ethyl-4-hydroxy-2H-indazole-6-carboxylate (I-1e-2) and azetidin-1-yl(3,5-difluoropyridin-2-yl)methanone (SM-7).

¹H NMR (400 MHz, CHLOROFORM-d) δ ppm 1.66 (t, 3H), 2.36 (quin, J=7.7 Hz, 2H), 3.95 (s, 3H), 4.27 (t, J=7.8 Hz, 2H), 4.35-4.41 (m, 2 H), 4.51 (q, J=7.4 Hz, 2H), 7.10 (dd, J=10.5, 2.3 Hz, 1H), 7.33 (d, J=1.0 Hz, 1H), 7.84 (d, J=0.8 Hz, 1H), 8.27-8.29 (m, 1H), 8.41 (t, 1H). MS (M+1): 399.5.

### Preparation of methyl 2-ethyl-4-(5-(pyrrolidine-1-carbonyl)pyrazin-2-yloxy)-2H-indazole-6-carboxylate (I-1f-18)

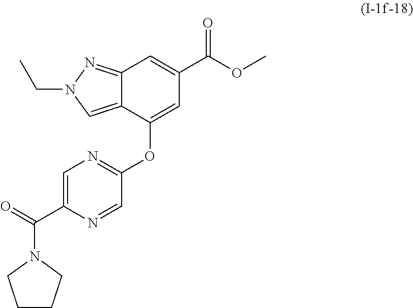

The title compound was prepared by a method analogous to that described for I-1f-5, using methyl 2-ethyl-4-hydroxy-2H-indazole-6-carboxylate (I-1e-2) and (5-chloropyrazin-2-yl)(pyrrolidin-1-yl)methanone (SM-1).

¹H NMR (400 MHz, CHLOROFORM-d) δ ppm 1.65 (t, 3H), 1.93-2.00 (m, 4H), 3.71 (t, J=6.8 Hz, 2H), 3.82 (t, J=6.6 Hz, 2H), 3.95 (s, 3H), 4.49 (q, J=7.4 Hz, 2H), 7.51 (d, J=1.0 Hz, 1H), 7.77 (d, J=1.0 Hz, 1H), 8.42 (d, J=1.4 Hz, 1H), 8.44 (t, J=1.1 Hz, 1H), 8.70 (d, J=1.4 Hz, 1H). MS (M+1): 396.0.

### Preparation of methyl 4-(6-(azetidine-1-carbonyl)-5-fluoropyridin-3-yloxy)-2-methyl-2H-indazole-6-carboxylate (I-1f-19)

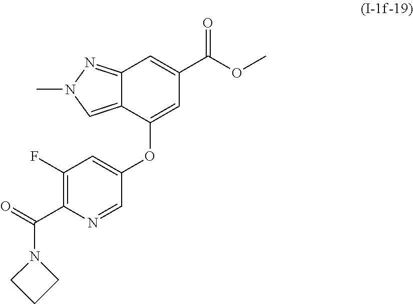

The title compound was prepared by a method analogous to that described for I-1f-5, using methyl 4-hydroxy-2-methyl-2H-indazole-6-carboxylate (I-1e-1) and azetidin-1-yl(3,5-difluoropyridin-2-yl)methanone (SM-7).

¹H NMR (400 MHz, CHLOROFORM-d) δ ppm 2.31-2.41 (m, 2H), 3.96 (s, 3H), 4.23-4.30 (m, 5H), 4.35-4.42 (m, 2H), 7.09 (dd, J=10.5, 2.3 Hz, 1H), 7.34 (d, J=1.0 Hz, 1H), 7.79 (s, 1H), 8.26-8.29 (m, 1H), 8.39 (t, J=1.1 Hz, 1H). MS (M+1): 385.5.

### Preparation of methyl 4-(2-(dimethylcarbamoyl)pyrimidin-5-yloxy)-2-ethyl-2H-indazole-6-carboxylate (I-1f-20)

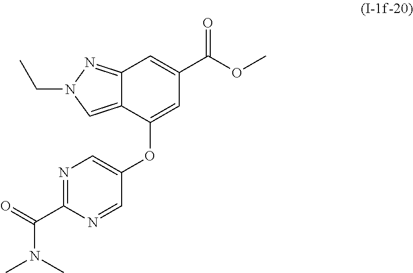

Methyl 2-ethyl-4-hydroxy-2H-indazole-6-carboxylate (I-1e-2: 60 mg, 0.27 mmol) was dissolved in dimethylformamide (2.0 mL) and a 60% dispersion of sodium hydride in mineral oil (16.3 mg, 0.408 mmol) was added. The mixture was stirred at room temperature for 15 minutes before adding 5-bromo-N,N-dimethylpyrimidine-2-carboxamide (SM-6: 75.0 mg, 0.326 mmol). The reaction was then heated to 100° C. for 5 days. The reaction was cooled to room temperature and partitioned between ethyl acetate and water. The layers were separated and the aqueous was extracted 2 more times with ethyl acetate. The combined organics were dried over magnesium sulfate, filtered, and concentrated. Purification by column chromatography (0-20% methanol in ethyl acetate) gave the desired methyl 4-(2-(dimethyl carbamoyl)pyrimidin-5-yloxy)-2-ethyl-2H-indazol e-6-carboxylate (I-1f-20: 21.2 mg, 21%) as a tan oil.

¹H NMR (400 MHz, CHLOROFORM-d) δ ppm 1.67 (t, 3H), 3.02 (s, 3 H), 3.18 (s, 3H), 3.95 (s, 3H), 4.52 (q, J=7.4 Hz, 2H), 7.28 (d, J=1.0 Hz, 1 H), 7.90 (d, 1H), 8.41 (t, 1H), 8.58 (s, 2H). MS (M+1): 370.5.

### Preparation of ethyl 4-(5-(azetidine-1-carbonyl)pyrazin-2-yloxy)-2-ethyl-2H-indazole-6-carboxylate (I-1f-21)

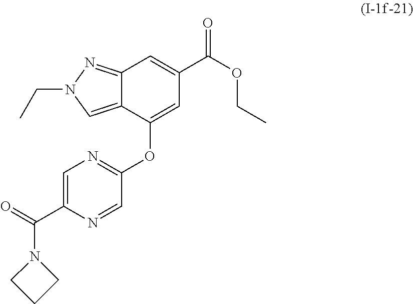

The title compound was prepared by a method analogous to that described for I-1f-5, using ethyl 2-ethyl-4-hydroxy-2H-indazole-6-carboxylate (I-1e-4) and azetidin-1-yl(5-chloropyrazin-2-yl)methanone (SM-8).

¹H NMR (400 MHz, CHLOROFORM-d) δ ppm 1.38 (m, 3H), 1.61 (t, J=7.32 Hz, 3H), 2.36 (quin, J=7.80 Hz, 2H), 4.24 (t, J=7.80 Hz, 2H), 4.34-4.49 (m, 4H), 4.66 (t, J=7.71 Hz, 2H), 7.48 (d, J=0.98 Hz, 1H), 7.71 (d, J=0.78 Hz, 1H), 8.36 (d, J=1.17 Hz, 1H), 8.42 (t, J=0.98 Hz, 1H), 8.82 (d, J=1.37 Hz, 1H). MS (M+1): 396.1.

### Preparation of ethyl 4-(4-(dimethylcarbamoyl)phenoxy)-2-methyl-2H-indazole-6-carboxylate (I-1f-22)

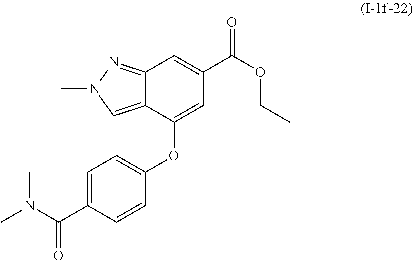

To a solution of ethyl 2-methyl-4-(trifluoromethylsulfonyloxy)-2H-indazole-6-carboxylate (I-1f-33: 220 mg, 0.624 mmol) and 4-hydroxy-N,N-dimethylbenzamide (113 mg, 0.686 mmol) in toluene (7.3 mL) was added potassium phosphate (265 mg, 1.25 mmol). After stirring under nitrogen for 5 minutes, a solution of palladium (II) acetate (21.1 mg, 0.094 mmol) and 2-di-t-butylphosphino-2′,4′,6′-tri-isopropyl-1,1′-biphenyl (79.4 mg, 0.187 mmol) in toluene (1.0 mL) was added to the mixture. The reaction was heated to 110° C. for 18 hours. The reaction was then concentrated, the crude dissolved in dichloromethane and washed with water. The organics were dried over magnesium sulfate, filtered, and concentrated. Purification by column chromatography, eluting with methanol in dichloromethane, gave the desired ethyl 4-(4-(dimethylcarbamoyl)phenoxy)-2-methyl-2H-indazole-6-carboxylate (I-1f-22: 27 mg, 12%) as a yellow solid.

MS (M+1): 367.9 RT 2.22 min.

### Preparation of methyl 4-(5-(azetidine-1-carbonyl)pyrazin-2-yloxy)-2-ethyl-2H-indazole-6-carboxylate (I-1f-23)

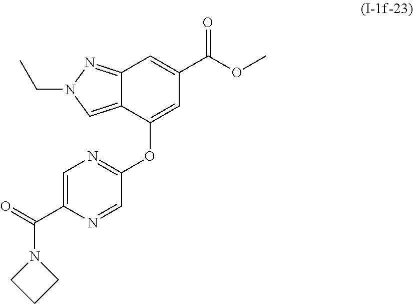

The title compound was prepared by a method analogous to that described for I-1f-5, using methyl 2-ethyl-4-hydroxy-2H-indazole-6-carboxylate (I-1e-2) and azetidin-1-yl(5-chloropyrazin-2-yl)methanone (SM-8).

¹H NMR (400 MHz, CHLOROFORM-d) δ ppm 1.64 (t, 3H), 2.34-2.45 (m, 2H), 3.95 (s, 3H), 4.27 (dd, J=8.2, 7.4 Hz, 2H), 4.49 (q, J=7.2 Hz, 2H), 4.70 (t, J=7.7 Hz, 2H), 7.51 (d, J=1.2 Hz, 1H), 7.75 (d, J=0.8 Hz, 1H), 8.40 (d, J=1.4 Hz, 1H), 8.43-8.46 (m, 1H), 8.85 (d, J=1.2 Hz, 1H). MS (M+1): 382.0.

### Preparation of methyl 4-(5-(azetidine-1-carbonyl)pyrazin-2-yloxy)-2-methyl-2H-indazole-6-carboxylate (I-1f-24)

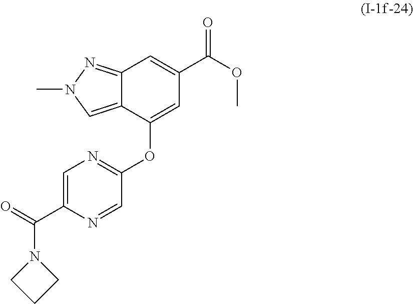

The title compound was prepared by a method analogous to that described for I-1f-5, using methyl 4-hydroxy-2-methyl-2H-indazole-6-carboxylate (I-1e-1) and azetidin-1-yl(5-chloropyrazin-2-yl)methanone (SM-8).

¹H NMR (400 MHz, CHLOROFORM-d) δ ppm 2.31-2.43 (m, 2H), 3.93 (s, 3H), 4.23 (s, 3H), 4.27 (br. s., 2H), 4.67 (br. s., 2H), 7.50-7.53 (m, 1H), 7.70 (s, 1H), 8.38 (d, J=1.17 Hz, 1H), 8.39-8.42 (m, 1H), 8.82 (d, J=1.37 Hz, 1H). MS (M+1): 368.4.

### Preparation of ethyl 4-(5-(azetidine-1-carbonyl)pyrazin-2-yloxy)-2-isopropyl-2H-indazole-6-carboxylate (I-1f-25)

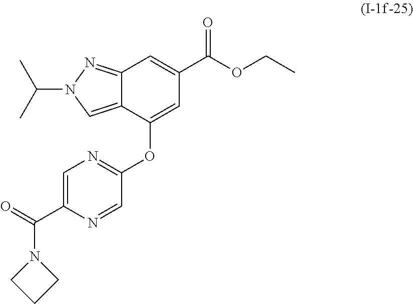

The title compound was prepared by a method analogous to that described for I-1f-5, using ethyl 4-hydroxy-2-isopropyl-2H-indazole-6-carboxylate (I-1e-5) and azetidin-1-yl(5-chloropyrazin-2-yl)methanone (SM-8).

¹H NMR (400 MHz, CHLOROFORM-d) δ ppm 1.38 (t, J=7.13 Hz, 3 H), 1.63 (d, J=6.64 Hz, 6H), 2.31-2.43 (m, 2H), 4.24 (t, 2H), 4.38 (q, J=7.17 Hz, 2H), 4.67 (t, 2H), 4.74-4.84 (m, 1H), 7.49 (s, 1H), 7.76 (s, 1 H), 8.37 (s, 1H), 8.45 (s, 1H), 8.83 (s, 1H). MS (M+1): 410.4.

### Preparation of ethyl 4-(5-(dimethylcarbamoyl)pyrazin-2-yloxy)-2-ethyl-2H-indazole-6-carboxylate (I-1f-26)

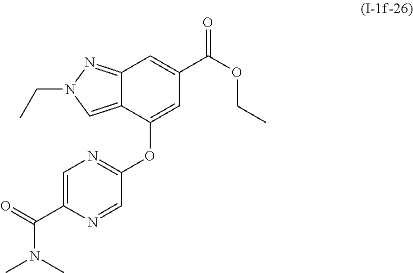

The title compound was prepared by a method analogous to that described for I-1f-5, using ethyl 2-ethyl-4-hydroxy-2H-indazole-6-carboxylate (I-1e-4) and 5-chloro-N,N-dimethylpyrazine-2-carboxamide (SM-3).

¹H NMR (400 MHz, CHLOROFORM-d) δ ppm 1.39 (t, 3H), 1.59-1.67 (m, 3H), 3.15 (d, J=9.56 Hz, 6H), 4.38 (q, J=7.22 Hz, 2H), 4.46 (q, J=7.35 Hz, 2H), 7.49 (d, J=0.98 Hz, 1H), 7.75 (s, 1H), 8.41 (d, J=1.37 Hz, 1H), 8.43 (s, 1H), 8.49 (d, J=1.37 Hz, 1H). MS (M+1): 384.1.

### Preparation of ethyl 4-(5-(dimethylcarbamoyl)pyrazin-2-yloxy)-2-isopropyl-2H-indazole-6-carboxylate (I-1f-27)

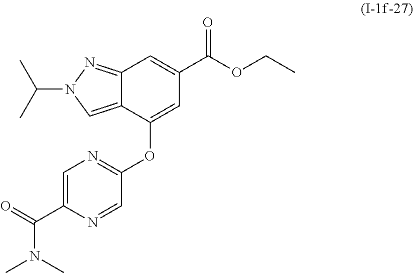

The title compound was prepared by a method analogous to that described for I-1f-5, using ethyl 4-hydroxy-2-isopropyl-2H-indazole-6-carboxylate (I-1e-5) and 5-chloro-N,N-dimethylpyrazine-2-carboxamide (SM-3).

¹H NMR (400 MHz, CHLOROFORM-d) δ ppm 1.38 (t, J=7.13 Hz, 3 H), 1.64 (d, J=6.64 Hz, 6H), 3.15 (d, J=8.79 Hz, 6H), 4.38 (q, J=7.04 Hz, 2 H), 4.73-4.85 (m, 1H), 7.49 (s, 1H), 7.79 (s, 1H), 8.41 (s, 1H), 8.45 (s, 1 H), 8.49 (s, 1H). MS (M+1): 398.4.

### Preparation of ethyl 4-(5-(bis(2,4-dimethoxybenzyl)carbamoyl)pyrazin-2-yloxy)-2-ethyl-2H-indazole-6-carboxylate (I-1f-28)

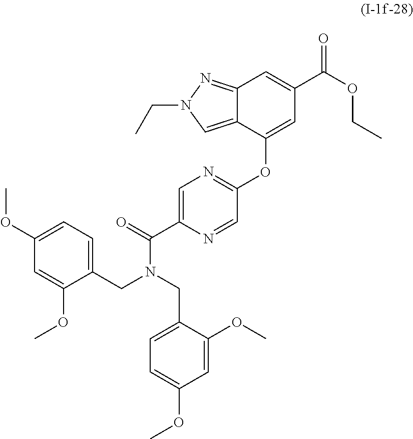

To a solution of ethyl 2-ethyl-4-hydroxy-2H-indazole-6-carboxylate (I-1e-4: 50 mg, 0.21 mmol) in dimethylformamide (1.06 mL) was added 5-chloro-N,N-bis(2,4-dimethoxybenzyl)pyrazine-2-carboxamide (SM-13: 97.5 mg, 0.213 mmol) and cesium carbonate (76.2 mg, 0.234 mmol). The reaction was heated to 110° C. for 5 hours. The reaction was diluted with 2 mL of ethyl acetate, washed with water (3×1 mL), dried over sodium sulfate, filtered, and concentrated. Column chromatography (10-100% ethyl acetate in heptane) afforded the desired ethyl 4-(5-(bis(2,4-dimethoxybenzyl)carbamoyl)pyrazin-2-yloxy)-2-ethyl-2H-indazole-6-carboxylate (I-1f-28: 107.9 mg, 77%).

¹H NMR (400 MHz, CHLOROFORM-d) δ ppm 1.39 (t, 3H) 1.62 (t, J=7.32 Hz, 3H) 3.61 (s, 3H) 3.76 (s, 3H) 3.77 (s, 3H) 3.79 (s, 3H) 4.38 (q, J=7.02 Hz, 2H) 4.46 (q, J=7.35 Hz, 2H) 4.62 (d, J=2.54 Hz, 4H) 6.35 (d, J=2.15 Hz, 1H) 6.40 (dd, J=8.29, 2.24 Hz, 1H) 6.44 (d, J=2.15 Hz, 1H) 6.47 (dd, J=8.39, 2.34 Hz, 1H) 7.01 (d, J=8.19 Hz, 1H) 7.28 (d, J=8.19 Hz, 1H) 7.49 (d, J=0.98 Hz, 1H) 7.71 (s, 1H) 8.38 (d, J=1.17 Hz, 1H) 8.43 (s, 1 H) 8.47 (d, J=1.37 Hz, 1H). MS (M+1): 656.4

### Preparation of 4-(5-(bis(2,4-dimethoxybenzyl)carbamoyl)pyrazin-2-yloxy)-2-ethyl-N-(5-methylpyridin-2-yl)-2H-indazole-6-carboxamide (I-1g-2)

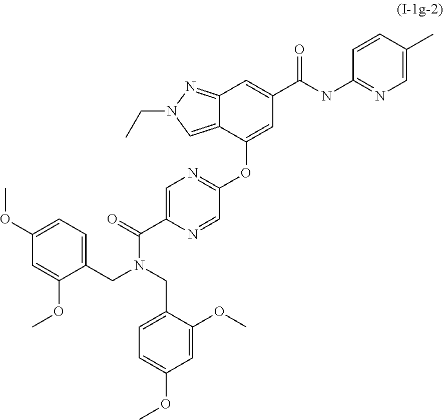

To a solution of 5-methylpyridin-2-amine (30.5 mg, 0.282 mmol) in 1,2-dimethoxyethane (0.5 mL) at 0° C. was added dimethylaluminum chloride (1.0M in hexanes) (0.564 mL, 0.564 mmol). The mixture was allowed to warm to room temperature and stir for 1 hour. The reaction mixture was then added to a solution of ethyl 4-(5-(bis(2,4-dimethoxybenzyl)carbamoyl)pyrazin-2-yloxy)-2-ethyl-2H-indazole-6-carboxylate (I-1f-28: 105 mg, 0.160 mmol) in 1,2-dimethoxyethane (0.9 mL). The reaction was heated to 80° C. for 4 hours. The reaction was then cooled to room temperature and methanol (0.5 mL) and saturated ammonium chloride (1.0 mL) were added. The mixture was stirred for 1 hour, then left standing overnight. The solution was filtered to remove the yellow solid that had formed. The filtrate was concentrated and some solid remained. This material was diluted with 10% methanol in dichloromethane and again filtered. The filtrate was concentrated and purified by column chromatography (10-100% ethyl acetate in heptane) to afford the title compound 4-(5-(bis(2,4-dimethoxybenzyl)carbamoyl)pyrazin-2-yloxy)-2-ethyl-N-(5-methylpyridin-2-yl)-2H-indazole-6-carboxamide (I-1g-2: 78.6 mg, 39%).

¹H NMR (400 MHz, CHLOROFORM-d) δ ppm 1.25 (t, J=7.22 Hz, 3 H), 1.63 (t, J=7.41 Hz, 3H), 2.31 (s, 3H), 3.77 (s, 6H), 3.80 (s, 3H), 4.47 (q, J=7.35 Hz, 2H), 4.62 (d, J=3.90 Hz, 4H), 6.35 (d, J=2.34 Hz, 1H), 6.40 (dd, J=8.29, 2.24 Hz, 1H), 6.44 (d, J=2.15 Hz, 1H), 6.47 (dd, J=8.19, 2.34 Hz, 1H), 7.01 (d, J=8.19 Hz, 1H), 7.28 (d, J=8.19 Hz, 1H), 7.43 (d, J=1.17 Hz, 1H), 7.56 (dd, J=8.49, 2.24 Hz, 1H), 7.73 (s, 1H), 8.12 (d, J=2.15 Hz, 1 H), 8.17 (s, 1H), 8.25 (d, J=8.58 Hz, 1H), 8.41 (d, J=1.37 Hz, 1H), 8.48 (d, J=1.37 Hz, 1H), 8.52 (s, 1H). MS (M+1): 718.7.

### Preparation of ethyl 4-[(1,1-dioxido-2,3-dihydro-1-benzothien-5-yl)oxy]-2-methyl-2H-indazole-6-carboxylate (I-1f-29)

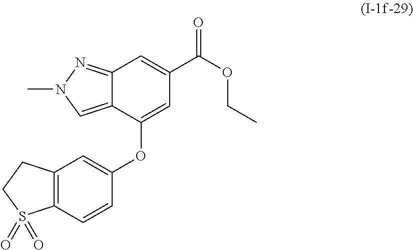

Ethyl 4-hydroxy-2-methyl-2H-indazole-6-carboxylate (I-1e-3: 100 mg, 0.454 mmol) was dissolved in dimethylformamide. Added 5-bromo-2,3-dihydro-bezo[b]thiophene dioxide (WO2004009086) (112 mg, 0.454 mmol), followed by cesium carbonate (296 mg, 0.908 mmol). Next added copper chloride (22.5 mg, 0.227 mmol) and 2,2,6,6-tetramethylheptane-3,5-dione (41.8 mg, 0.227 mmol). Added another equivalent of cesium carbonate (148 mg, 0.454 mmol) and heated the reaction to 120° C. in a sealed tube for 18 hours. The reaction was cooled to room temperature and diluted with water (1.0 mL) and ethyl acetate (5 mL). The organic layer was separated, washed with water, dried over sodium sulfate, filtered and concentrated. Purification by column chromatography (0-5% methanol in dichloromethane) afforded the title compound ethyl 4-[(1,1-dioxido-2,3-dihydro-1-benzothien-5-yl)oxy]-2-methyl-2H-indazole-6-carboxylate (I-1f-29: 98 mg, 56%) as a yellow solid.

¹H NMR (400 MHz, CHLOROFORM-d) δ ppm 1.41 (t, J=7.13 Hz, 3 H), 3.31 (t, J=6.93 Hz, 2H), 3.51 (t, 2H), 4.22 (s, 3H), 4.40 (q, J=7.03 Hz, 2 H), 6.91 (d, J=1.95 Hz, 1H), 7.13 (dd, J=8.59, 2.15 Hz, 1H), 7.31 (d, J=0.98 Hz, 1H), 7.62-7.78 (m, 2H), 8.37 (s, 1H). MS (M+1): 387.2.

### Preparation of ethyl 4-[(1,1-dioxido-2,3-dihydro-1-benzothien-5-yl)oxy]-2-ethyl-2H-indazole-6-carboxylate (I-1f-30)

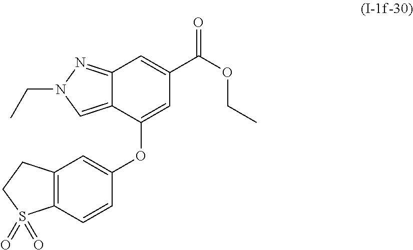

The title compound was prepared by a method analogous to that described for (I-1f-29), using ethyl 2-ethyl-4-hydroxy-2H-indazole-6-carboxylate (I-1e-4).

¹H NMR (400 MHz, CHLOROFORM-d) δ ppm 1.40 (t, J=7.13 Hz, 3 H), 1.64 (t, J=7.32 Hz, 3H), 3.31 (t, J=6.93 Hz, 2H), 3.52 (t, J=6.93 Hz, 2 H), 4.40 (q, J=7.22 Hz, 2H), 4.48 (q, J=7.35 Hz, 2H), 6.92 (d, J=1.56 Hz, 1 H), 7.14 (dd, J=8.59, 1.95 Hz, 1H), 7.30 (s, 1H), 7.71 (d, J=8.59 Hz, 1H), 7.78 (s, 1H), 8.39 (s, 1H). MS (M+1): 401.3.

### Preparation of methyl 2-ethyl-4-(4-(methylsulfonyl)phenoxy)-2H-indazole-6-carboxylate (I-1f-31)

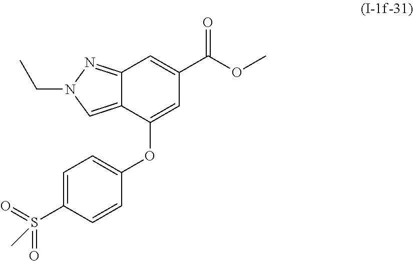

To a solution of methyl 2-ethyl-4-hydroxy-2H-indazole-6-carboxylate (I-1e-2: 2.0 g, 9.1 mmol) in dimethylformamide (20 mL) was added 1-fluoro-4-(methylsulfonyl)benzene (1.90 g, 10.9 mmol) and cesium carbonate (5.92 g, 18.2 mmol). The mixture was stirred at 100° C. for 18 hours. The reaction mixture was diluted with water (10 mL) and extracted with EtOAc. The combined organic layers were dried and concentrated under reduced pressure. Column chromatography (2-7% methanol in dichloromethane) gave the title compound methyl 2-ethyl-4-(4-(methylsulfonyl)phenoxy)-2H-indazole-6-carboxylate (I-1f-31: 1.93 g, 57%) as a white solid.

¹HNMR (400 MHz, DMSO-d6) δ ppm 1.49-1.52 (t, 3H), 3.24 (s, 3H), 3.87 (s, 3H), 4.47-4.52 (q, 2H), 7.07 (d, 1H), 7.30-7.32 (d, 2H), 7.95-7.97 (d, 2H), 8.19 (s, 1H), 8.50 (s, 1H).

### Preparation of methyl 4-(4-(N,N-bis(2,4-dimethoxybenzyl)sulfamoyl)phenoxy)-2-ethyl-2H-indazole-6-carboxylate (I-1f-32)

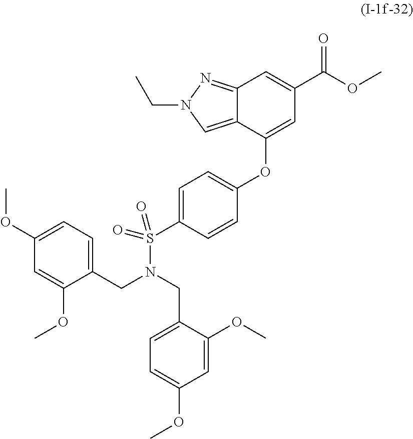

The title compound was prepared by a method analogous to that described for (I-1f-31), using N,N-bis(2,4-dimethoxybenzyl)-4-fluorobenzenesulfonamide (SM-15).

¹H NMR (400 MHz, CHLOROFORM-d) δ ppm 1.54-1.57 (m, 3H), 3.68 (s, 6H), 3.73 (s, 6H), 3.86 (s, 3H), 4.32 (s, 4H), 4.37-4.43 (m, 2H), 6.22 (d, 2H), 6.30-6.32 (d, 2H), 6.93-6.95 (d, 2H), 7.07-7.09 (d, 2H), 7.17-7.19 (d, 1H), 7.56-7.58 (d, 2H), 7.74 (s, 1H), 8.26 (s, 1H).

### Preparation of ethyl 2-methyl-4-(trifluoromethylsulfonyloxy)-2H-indazole-6-carboxylate (I-1f-33)

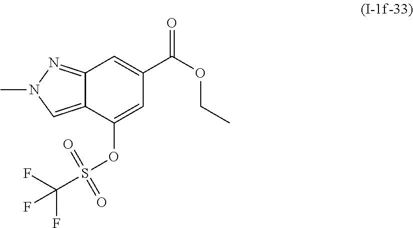

To a stirred solution of ethyl 4-hydroxy-2-methyl-2H-indazole-6-carboxylate (I-1e-3: 220.2 mg, 1.000 mmol) and N,N-diisopropylethylamine (0.261 mL, 1.50 mmol) in dichloromethane (5.0 mL) at 0° C., was added a solution of 1,1,1-trifluoro-N-phenyl-N-(trifluoromethylsulfonyl)methanesulfonamide (429 mg, 1.20 mmol) in 1 mL of dichloromethane. The reaction was left to stir and gradually warm to room temperature over 16 hours. The reaction mixture was then concentrated and purified by column chromatography (50-100% ethyl acetate in heptane) to afford the title compound ethyl 2-methyl-4-(trifluoromethylsulfonyloxy)-2H-indazole-6-carboxylate (I-1f-33: 220 mg, 62%) as a white solid.

¹H NMR (400 MHz, CHLOROFORM-d) δ ppm 1.42 (t, 3H) 4.29 (s, 3 H) 4.42 (q, 2H) 7.63 (s, 1H) 8.04 (s, 1H) 8.50 (d, J=0.98 Hz, 1H). MS (M+1): 352.8.

### Preparation of ethyl 2-ethyl-4-[4-(methylsulfonyl)phenoxy]-2H-indazole-6-carboxylate (I-1f-34)

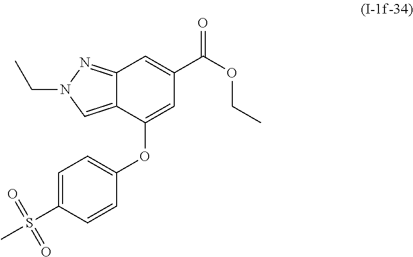

The title compound was prepared by heating a mixture of ethyl 2-ethyl-4-hydroxy-2H-indazole-6-carboxylate (I-1e-4, 200 mg, 0.854 mmol), 4-fluorophenyl methylsulfone (178 mg, 1.02 mmol) and cesium carbonate (334 mg, 2.85 mmol) in N,N-dimethylformamide (2.85 mL) at 80° C. overnight. To the mixture was added additional 4-fluorophenyl methylsulfone (178 mg, 1.02 mmol) and potassium carbonate (236 mg, 1.71 mmol). This mixture was then heated to 90° C. overnight. The reaction mixture was then cooled, diluted with ethyl acetate and washed 3 times with water. The organic layer was dried over sodium sulfate, filtered and concentrated. The crude product was purified by flash chromatography eluting with a 10-100% gradient of ethyl acetate in heptane to afford ethyl 2-ethyl-4-[4-(methylsulfonyl)phenoxy]-2H-indazole-6-carboxylate (I-1f-34, 61 mg, 18%)

¹H NMR (400 MHz, CHLOROFORM-d) ppm 1.39 (t, J=7.12 Hz, 3 H), 1.63 (t, J=7.32 Hz, 3H), 3.07 (s, 3H), 4.38 (q, 2H), 4.48 (q, 2H), 7.14 (s, 1H), 7.17 (s, 1H), 7.31 (s, 1H), 7.79 (s, 1H), 7.89 (s, 1H), 7.92 (s, 1H), 8.38 (s, 1H). MS (M+1): 389.1.

### Preparation of 2-ethyl-4-[4-(methylsulfonyl)phenoxy]-2H-indazole-6-carboxylic acid (I-1g-3)

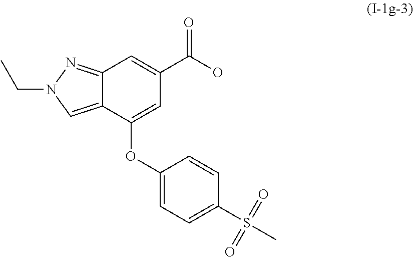

A solution of ethyl 2-ethyl-4-[4-(methylsulfonyl)phenoxy]-2H-indazole-6-carboxylate (I-1f-34, 32 mg, 0.082 mmol) and 1M LiOH solution (0.246 mL) in 1,4-dioxane (0.273 mL), was heated to 50° C. for 2 hours. The pH of the reaction mixture was adjusted to between 4 and 5 with 1N HCl and extracted with ethyl acetate. The organic solution was washed with water and dried over sodium sulfate to afford the title compound 2-ethyl-4-[4-(methylsulfonyl)phenoxy]-2H-indazole-6-carboxylic acid (I-1g-3: 6 mg, 10%).

¹H NMR (400 MHz, METHANOL-d4) ppm 2.28 (t, 3H), 4.00 (s, 3 H), 5.27 (t, 2H), 7.84 (d, J=0.78 Hz, 1H), 8.04-8.14 (m, 2H), 8.68-8.78 (m, 2H), 8.94 (s, 1H), 9.26 (s, 1H), 13.84 (br. s., 1H). MS (M+1): 389.1.

### Preparation of ethyl 4-{4-[(dimethylamino)sulfonyl]phenoxy}-2-methyl-2H-indazole-6-carboxylate (I-1f-35)

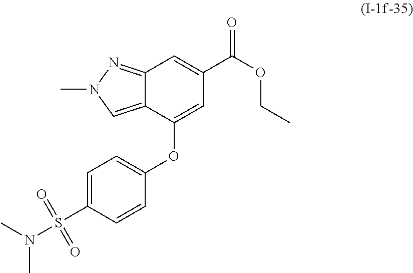

The title compound was prepared by heating a mixture of ethyl 4-hydroxy-2-methyl-2H-indazole-6-carboxylate (I-1e-3, 70 mg, 0.32 mmol), 4-fluoro-N,N-dimethylbenzenesulfonamide (71.1 mg, 0.35 mmol), and cesium carbonate (114 mg, 0.35 mmol) in N,N-dimethylformamide (0.795 mL) at 100° C. overnight. The reaction mixture was then cooled, concentrated, dissolved in water, and extracted three times with dichloromethane. The combined organic layers were concentrated and the crude product was purified by flash chromatography eluting with a 20-65% gradient of ethyl acetate in heptane to afford ethyl 4-{4-[(dimethylamino)sulfonyl]phenoxy}-2-methyl-2H-indazole-6-carboxylate (I-1f-35, 68 mg, 53%).

¹H NMR (400 MHz, CHLOROFORM-d) ppm 1.39 (t, J=7.13 Hz, 3 H), 2.72 (s, 6H), 4.22 (s, 3H), 4.39 (q, 2H), 7.13 (d, J=8.99 Hz, 2H), 7.31 (d, J=1.17 Hz, 1H), 7.69-7.78 (m, 3H), 8.35 (s, 1H). MS (M+1) 403.8.

### Preparation of ethyl 2-methyl-4-{4-[(methylamino)sulfonyl]phenoxy}-2H-indazole-6-carboxylate (I-1f-36)

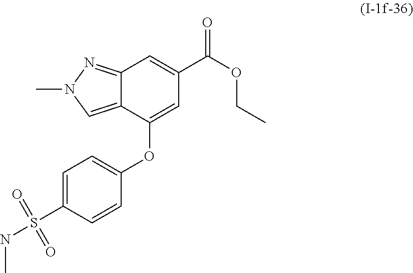

The title compound was prepared by a method analogous to that described for I-1f-35, using 4-fluoro-N-methylbenzenesulfonamide.

¹H NMR (400 MHz, CHLOROFORM-d) ppm 1.39 (t, J=7.13 Hz, 3 H), 2.69 (d, J=5.28 Hz, 3H), 4.22 (s, 3H), 4.24-4.32 (m, 1H), 4.38 (q, J=7.04 Hz, 2H), 7.09-7.12 (m, 1H), 7.12-7.14 (m, 1H), 7.30 (d, J=0.98 Hz, 1H), 7.74 (s, 1H), 7.79-7.82 (m, 1H), 7.82-7.85 (m, 1H), 8.34 (t, J=0.98 Hz, 1H). MS (M+1) 390.1.

### Preparation of methyl 4-(4-{[bis(2,4-dimethoxybenzyl)amino]sulfonyl}phenoxy)-2-methyl-2H-indazole-6-carboxylate (I-1f-37)

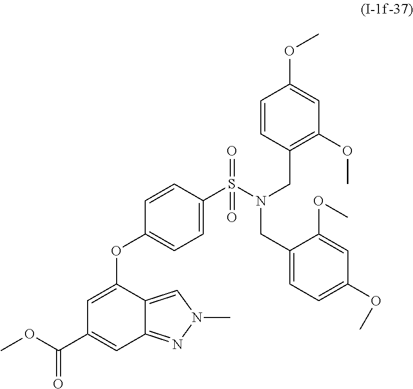

A flask was charged with methyl 4-hydroxy-2-methyl-2H-indazole-6-carboxylate (I-1e-1: 309 mg, 1.50 mmol), N,N-bis(2,4-dimethoxybenzyl)-4-iodobenzenesulfonamide (SM-14: 875 mg, 1.50 mmol), cesium carbonate (740 mg, 2.2 mmol) and 2,2,6,6-tetramethyl 3,5-heptanedione (141 mg, 0.75 mmol). 1-Methyl-2-pyrrolidinone (3 mL) was added to the flask and the mixture was purged by bubbling nitrogen with stirring for 15 minutes. Copper (I) chloride (75 mg, 0.75 mmol) was added and the mixture was heated to 110° C. under an atmosphere of nitrogen overnight. The mixture was partitioned between ethyl acetate and concentrated ammonium chloride solution, and suction filtered to remove some insoluble material. The filtrate was separated, and the aqueous phase was extracted with ethyl acetate. The organic solution was washed with brine, dried over sodium sulfate, filtered, evaporated, and flash chromatographed eluting with 75% ethyl acetate in heptane to afford the desired methyl 4-(4-{[bis(2,4-dimethoxybenzyl)amino]sulfonyl}phenoxy)-2-methyl-2H-indazole-6-carboxylate (I-1f-37: 506 mg, 51%).

¹H NMR (400 MHz, CHLOROFORM-d) δ 3.61 (s, 6H), 3.74 (s, 6H), 3.93 (s, 3H), 4.21 (s, 3H), 4.38 (s, 4H), 6.28 (s, 2H), 6.37 (dd, 2H, J=8.0, 2.0), 6.98 (d, 2H, J=8.8), 7.14 (d, 2H, J=8.4), 7.25 (s, 1H), 7.62 (d, 2H, J=8.8), 7.72 (s, 1H), 8.31 (s, 1H). MS (M+23) 684.4.

### Preparation of methyl 4-[4-(aminosulfonyl)phenoxy]-2-methyl-2H-indazole-6-carboxylate (I-1g-4)

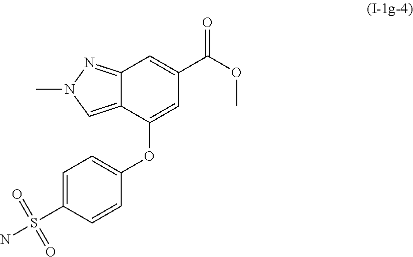

To a stirred solution of I-1f-37 (506 mg, 0.765 mmol) in dichloromethane (4 mL) was added trifluoroacetic acid (0.5 mL) and triethylsilane (0.37 mL). The resulting mixture was stirred at room temperature for 4 hours. The mixture was diluted with dichloromethane, and saturated sodium bicarbonate was added. There was considerable material not in solution. 1N HCl was added to neutrality. The solids were collected by vacuum filtration, washed with water and dichloromethane, and dried under high vacuum to afford the title compound methyl 4-[4-(aminosulfonyl)phenoxy]-2-methyl-2H-indazole-6-carboxylate (I-1g-4: 253 mg, 91%).

¹H NMR (400 MHz, DMSO-d₆) ppm 3.82 (s, 3H), 4.18 (s, 3H), 6.95 (d, J=1.17 Hz, 1H), 7.16-7.34 (m, 4H), 7.76-7.88 (m, 2H), 8.11 (s, 1H), 8.41 (s, 1H). MS (M+1) 362.1.

### Preparation of methyl 4-({6-[(dimethylamino)sulfonyl]pyridin-3-yl}oxy)-2-methyl-2H-indazole-6-carboxylate (I-1f-38)

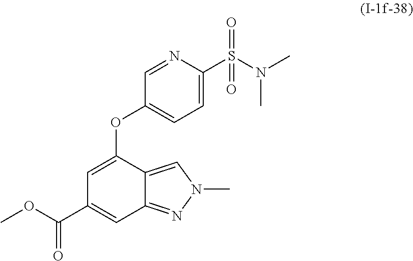

The title compound was prepared by a method analogous to that described for I-1f-37, using 5-bromo-N,N-dimethylpyridine-2-sulfonamide (WO 2008002244).

¹H NMR (400 MHz, CHLOROFORM-d) δ ppm 2.70 (s, 6H), 3.93 (s, 3H), 4.21 (s, 3H), 7.11 (d, 1H, J=8.4), 7.49 (s, 1H), 7.72 (s, 1H), 8.06 (dd, 1H, J=8.4, 2.4), 8.40 (s, 1H), 8.52 (s, 1H). MS (M+1): 391.1

### Preparation of methyl 4-({5-[(ethylsulfonyl)(methyl)amino]pyrazin-2-yl}oxy)-2-methyl-2H-indazole-6-carboxylate (I-1f-39)

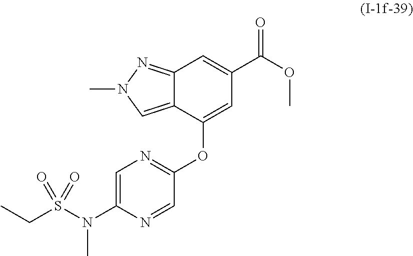

The title compound was prepared by a method analogous to that described for I-1f-37 using N-(5-chloropyrazin-2-yl)-N-methylethanesulfonamide (SM-9).

LCMS (M+1) 406.2, RT 1.17 min

### Preparation of methyl 2-methyl-4-{[6-(methylsulfonyl)pyridin-3-yl]oxy}-2H-indazole-6-carboxylate (I-1f-40)

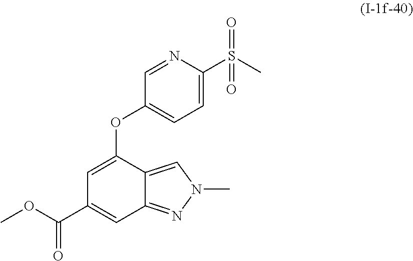

To a solution of methyl 4-hydroxy-2-methyl-2H-indazole-6-carboxylate (I-1e-1: 155 mg, 0.75 mmol) and potassium carbonate (207 mg, 1.5 mmol) in N,N-dimethylformamide (1.0 mL) was added 5-bromo-2-(methylsulfonyl)pyridine (177 mg, 0.75 mmol). The reaction was heated to 120° C. overnight, then cooled to room temperature and concentrated. The crude reaction mixture was partitioned between ethyl acetate and water. The layers were separated, the organics washed with brine, dried over sodium sulfate, filtered, and concentrated. Purification by column chromatography (50-80% ethyl acetate in heptane) afforded the title compound methyl 2-methyl-4-{[6-(methylsulfonyl)pyridin-3-yl]oxy}-2H-indazole-6-carboxylate (I-1f-40: 81 mg, 30%).

¹H NMR (400 MHz, CHLOROFORM-d) δ ppm 3.21 (s, 3H), 3.92 (s, 3 H), 4.22 (s, 3H), 7.30 (s, 1H), 7.42 (dd, 1H, J=8.4, 2.4), 7.78 (s, 1H), 8.02 (d, 1H, J=8.8), 8.36 (s, 1H), 8.49 (s, 1H). MS (M+1) 362.0.

### Preparation of ethyl 2-methyl-4-[4-(methylsulfonyl)-2-(trifluoromethyl)phenoxy]-2H-indazole-6-carboxylate (I-1f-41)

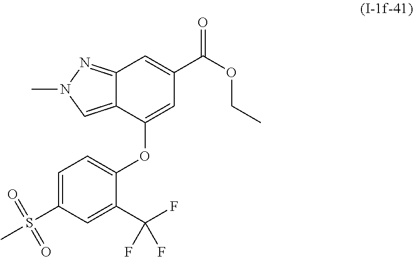

To a solution of ethyl 4-hydroxy-2-methyl-2H-indazole-6-carboxylate (I-1e-3: 66 mg, 0.30 mmol), copper (I) iodide (11.4 mg, 0.06 mmol), and cesium carbonate (117 mg, 0.36 mmol) in N,N-dimethylformamide (1.2 mL) was added 1-chloro-4-(methylsulfonyl)-2-(trifluoromethyl)benzene (93.1 mg, 0.36 mmol). The reaction was heated to 90° C. overnight, then cooled to room temperature and purified by column chromatography (20-80% ethyl acetate in heptane) to afford the title compound ethyl 2-methyl-4-[4-(methylsulfonyl)-2-(trifluoromethyl)phenoxy]-2H-indazole-6-carboxylate (I-1f-41: 13 mg, 10%).

¹H NMR (400 MHz, METHANOL-d4) ppm 2.28 (t, 3H), 4.00 (s, 3 H), 5.27 (t, 2H), 7.84 (d, J=0.78 Hz, 1H), 8.04-8.14 (m, 2H), 8.68-8.78 (m, 2H), 8.94 (s, 1H), 9.26 (s, 1H), 13.84 (br. s., 1H). MS (M+1) 442.8.

### Preparation of ethyl 4-{4-[N-(tert-butoxycarbonyl)-S-methylsulfonimidoyl]phenoxy}-2-ethyl-2H-indazole-6-carboxylate (I-1f-42)

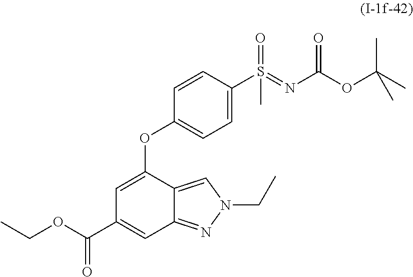

The title compound was prepared by mixing ethyl 2-ethyl-4-hydroxy-2H-indazole-6-carboxylate (I-1e-4, 100 mg, 0.42 mmol), tert-butyl [(4-bromophenyl)(methyl)oxido-lambda-4-sulfanylidene]carbamate (JOC 2005, 70, 2346-2349; 171 mg, 0.23712 mmol), cesium carbonate (211 mg, 0.640 mmol) and 2,2,6,6-tetramethyl 3,5-heptanedione (40 mg, 0.21 mmol) in 1-methyl-2-pyrrolidinone (2.0 mL). The mixture was purged with nitrogen. Copper (I) chloride (22 mg, 0.22 mmol) was added and the mixture was heated to 120° C. under an atmosphere of nitrogen for 6 hours. The mixture was partitioned between ethyl acetate and water. The organic layer was washed with water (3 times, slightly acidic) and brine, dried over sodium sulfate, filtered, evaporated, and purified by preparative thin layer chromatographed eluting with 90% ethyl acetate in heptane to afford ethyl 4-{4-[N-(tert-butoxycarbonyl)-S-methylsulfonimidoyl]phenoxy}-2-ethyl-2H-indazole-6-carboxylate (I-1f-42: 174 mg, 84%).

¹H NMR (400 MHz, CHLOROFORM-d) δ ppm 1.41 (t, 3H), 1.43 (s, 9 H), 1.64 (t, 3H), 3.28 (s, 2H), 4.41 (q, 2H), 4.48 (q, 2H), 7.21-7.16 (m, 2 H), 7.34 (d, 1H), 7.80 (d, 1H), 7.96-7.92 (m, 2H), 8.40-8.39 (m, 1H).

### Preparation of 2-ethyl-4-hydroxy-N-(5-methylpyridin-2-yl)-2H-indazole-6-carboxamide (I-2f-1)

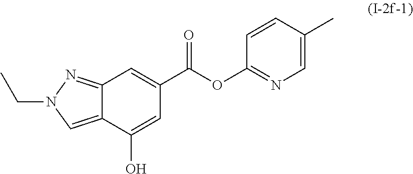

To a solution of 5-methylpyridin-2-amine (74.0 mg, 0.684 mmol) in 1,2-dimethoxyethane (1.0 mL) at 0° C., was added dimethylaluminum chloride (1.0M in hexanes) (1.37 mL, 1.37 mmol). After stirring at room temperature for 0.5 hour, this mixture was added to a solution of ethyl 2-ethyl-4-hydroxy-2H-indazole-6-carboxylate (I-1e-4: 80 mg, 0.34 mmol) in 1,2-dimethoxyethane (1.0 mL) and heated at 85° C. for 16 hours. The reaction was cooled to room temperature and methanol (2 mL) and saturated ammonium chloride (3 mL) were added. The solution was stirred for 1 hour and left standing overnight. The mixture was concentrated to remove methanol and the remaining solution was extracted with dichloromethane (5×2 mL). The combined organics were dried over sodium sulfate, filtered, and concentrated. Purification by column chromatography (10-50% ethyl acetate in heptane) gave the title compound 2-ethyl-4-hydroxy-N-(5-methylpyridin-2-yl)-2H-indazole-6-carboxamide (I-2f-1: 27 mg, 27%).

¹H NMR (400 MHz, CHLOROFORM-d) δ ppm 1.62 (t, J=7.41 Hz, 3 H), 2.31 (s, 3H), 4.46 (q, J=7.35 Hz, 2H), 6.95 (d, J=0.98 Hz, 1H), 7.56 (dd, J=8.58, 1.95 Hz, 1H), 7.77 (s, 1H), 8.05 (s, 1H), 8.12 (s, 1H), 8.25 (d, J=8.39 Hz, 1H), 8.78 (br. s., 1H). MS (M+1): 297.3.

### Example 1

### Preparation of 2-methyl-N-(5-methylpyridin-2-yl)-4-(5-(pyrrolidine-1-carbonyl)pyrazin-2-yloxy)-2H-indazole-6-carboxamide (1A)

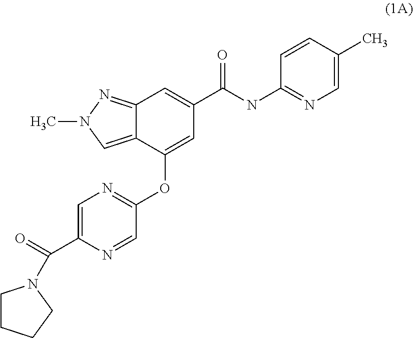

To a solution of 5-methylpyridin-2-amine (59.7 mg, 0.55 mmol) in 1,2-dimethoxyethane (0.5 mL), was added dimethylaluminum chloride (1.0M in hexanes) (1.10 mL). After stirring at room temperature for 0.5 hour, this mixture was added to a solution of methyl 2-methyl-4-(5-(pyrrolidine-1-carbonyl)pyrazin-2-yloxy)-2H-indazole-6-carboxylate (I-1f-2: 70 mg, 0.18 mmol) in 1,2-dimethoxyethane (1.30 mL) and heated at 90° C. for 16 hours. The reaction was cooled to room temperature and diluted with ethyl acetate and aqueous Rochelles salt. After stirring for 1 hour, the phases were separated and the aqueous phase was extracted two more times with ethyl acetate. The combined organics were dried over sodium sulfate, filtered, and concentrated. The crude was purified by column chromatography eluting with a gradient of 0-10% methanol in ethyl acetate to afford the title compound 2-methyl-N-(5-methylpyridin-2-yl)-4-(5-(pyrrolidine-1-carbonyl)pyrazin-2-yloxy)-2H-indazole-6-carboxamide (1A: 64 mg, 76%) as a yellow solid.

¹H NMR (400 MHz, CHLOROFORM-d) δ ppm 1.86-2.01 (m, 4H), 2.31 (s, 3H), 3.60-3.74 (m, 2H), 3.74-3.86 (m, 2H), 4.23 (s, 3H), 7.44 (d, J=1.17 Hz, 1H), 7.56 (dd, J=8.78, 2.15 Hz, 1H), 7.73 (s, 1H), 8.12 (d, J=2.34 Hz, 1H), 8.18 (s, 1H), 8.26 (d, J=8.58 Hz, 1H), 8.42 (d, J=1.37 Hz, 1H), 8.58 (s, 1H), 8.68 (d, J=1.17 Hz, 1H). MS (M+1): 458.1.

The compounds listed in Table 1 below were prepared using procedures analogous to those for the preparation of Compound (1A) above using the appropriate starting materials and intermediates described above.

TABLE 1

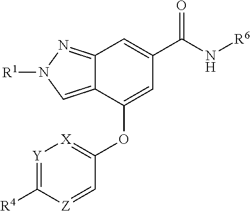

| Example | | | | | | |
|---|---|---|---|---|---|---|
| No. | R¹ | X | Y | R⁴ | Z | R⁶ |
| 1B | —CH₃ | CH | N | 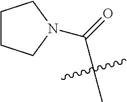 | N | pyrazin-2-yl |
| | ¹H NMR (400 MHz, CHLOROFORM-d) δ ppm 1.88-2.01 (m, 4 H), 3.69 (s, 2 H), 3.82 (t, 2 H), 4.24 (s, 3 H), 7.46 (d, J = 1.17 | | | | | |
| | Hz, 1 H), 7.75 (s, 1 H), 8.19 (t, J = 1.07 Hz, 1 H), 8.29 (dd, J = 2.73, 1.56 Hz, 1 H), 8.39 (d, J = 2.54 Hz, 1 H), 8.45 (d, J = 1.37 | | | | | |
| | Hz, 1 H), 8.55 (s, 1 H), 8.69 (d, J = 1.37 Hz, 1 H), 9.70 (d, J = 1.56 Hz, 1 H). MS (M + 1): 445.1 | | | | | |
| 1C | —CH₃ | CH | CH | 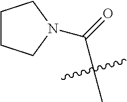 | CF | 5-methyl- pyridin-2-yl |
| | MS (M + 1): 474.1, retention time: 1.88 minutes | | | | | |
| 1D | —CH₃ | CH | CH | 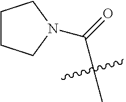 | CF | pyrazin-2-yl |
| | ¹H NMR (400 MHz, CHLOROFORM-d) δ ppm 1.84-2.02 (m, 4 H), 3.36 (t, J = 6.54 Hz, 2 H), 3.63 (t, J = 6.93 Hz, 2 H), 4.23 | | | | | |
| | (s, 3 H), 6.78 (dd, J = 10.54, 2.15 Hz, 1 H), 6.89 (dd, J = 8.49, 2.44 Hz, 1 H), 7.21 (d, J = 1.17 Hz, 1 H), 7.37-7.45 (m, 1 H), | | | | | |
| | 7.80 (s, 1 H), 8.09 (s, 1 H), 8.28 (dd, J = 2.73, 1.56 Hz, 1 H), 8.37 (d, J = 2.54 Hz, 1 H), 8.63 (s, 1 H), 9.68 (d, J = 1.56 Hz, 1 H). | | | | | |
| | MS (M + 1): 461.1 | | | | | |
| 1E | —CH₃ | CH | N | —C(O)N(CH₃)₂ | N | pyrazin-2-yl |
| | ¹H NMR (400 MHz, CHLOROFORM-d) δ ppm 3.14 (s, 3 H), 3.17 (s, 3 H), 4.24 (s, 3 H), 7.47 (s, 1 H), 7.76 (s, 1 H), 8.20 (s, 1 H), | | | | | |
| | 8.28 (br. s., 1 H), 8.38 (d, J = 1.95 Hz, 1 H), 8.45 (s, 1 H), 8.49 (s, 1 H), 8.65 (br. s., 1 H), 9.69 (s, 1 H). MS (M + 1): 419.3 | | | | | |
| 1F | —CH₃ | CH | N | —C(O)N(CH₃)₂ | N | 5-methyl- |
| | | | | | | pyrazin-2-yl |
| | ¹H NMR (400 MHz, CHLOROFORM-d) δ ppm 2.54 (s, 3 H), 3.14 (s, 3 H), 3.17 (s, 3 H), 4.23 (s, 3 H), 7.45 (s, 1 H), 7.76 (s, 1 H), | | | | | |
| | 8.14 (s, 1 H), 8.18 (s, 1 H), 8.45 (d, J = 1.17 Hz, 1 H), 8.49 (s, 1 H), 8.60 (s, 1 H), 9.56 (d, J = 0.98 Hz, 1 H). MS (M + 1): 433.1 | | | | | |
| 1G | —CH₃ | CH | N | —C(O)N(CH₃)₂ | N | 5-methyl- |
| | | | | | | pyridin-2-yl |
| | ¹H NMR (400 MHz, CHLOROFORM-d) δ ppm 2.31 (s, 3 H), 3.15 (s, 3 H), 3.17 (s, 3 H), 4.23 (s, 3 H), 7.45 (d, J = 0.98 Hz, | | | | | |
| | 1 H), 7.57 (dd, J = 8.50, 2.05 Hz, 1 H), 7.74 (s, 1 H), 8.12 (s, 1 H), 8.18 (s, 1 H), 8.26 (d, J = 8.40 Hz, 1 H), 8.44 (d, J = 1.37 | | | | | |
| | Hz, 1 H), 8.49 (d, J = 1.17 Hz, 1 H), 8.62 (s, 1 H). MS (M + 1): 432.1 | | | | | |
| 1H | —CH₃ | CH | N | —C(O)N(CH₃)₂ | N | 5-methoxy- |
| | | | | | | pyrazin-2-yl |
| | ¹H NMR (400 MHz, CHLOROFORM-d) δ ppm 3.15 (d, J = 9.97 Hz, 6 H), 3.97 (s, 3 H), 4.22 (s, 3 H), 7.45 (br. s., 1 | | | | | |
| | H), 7.75 (br. s., 1 H), 7.94 (br. s., 1 H), 8.16 (br. s., 1 H), 8.42 (s, 1 H), 8.48 (br. s., 2 H), 9.18 (br. s., 1 H). MS (M + 1): 449.3 | | | | | |
| 1I | —CH₃ | CH | N | —CF₃ | N | 5-methyl- |
| | | | | | | pyrazin-2-yl |
| | ¹H NMR (400 MHz, CHLOROFORM-d) δ ppm 2.54 (s, 3 H), 4.24 (s, 3 H), 7.49 (d, J = 0.98 Hz, 1 H), 7.79 (s, 1 H), 8.13 (s, | | | | | |
| | 1 H), 8.20 (s, 1 H), 8.42 (s, 1 H), 8.60 (d, J = 0.98 Hz, 1 H), 8.65 (s, 1 H), 9.56 (s, 1 H). MS (M + 1): 430.1 | | | | | |
| 1J | —CH₃ | CH | N | —CF₃ | N | 1-methyl-1H- |
| | | | | | | pyrazol-3-yl |
| | ¹H NMR (400 MHz, CHLOROFORM-d) δ ppm 3.82 (s, 3 H), 4.20 (s, 3 H), 6.60 (s, 1 H), 7.47 (br. s., 1 H), 7.51 (s, 1 H), | | | | | |
| | 8.13 (br. s., 1 H), 8.20 (br. s., 1 H), 8.49 (br. s., 1 H), 8.68 (s, 1 H). MS (M + 1): 418.0 | | | | | |
| 1K | —CH₃ | CH | N | —CF₃ | N | 2-methyl-2H- |
| | | | | | | 1,2,3-triazol-4-yl |
| | ¹H NMR (400 MHz, DMSO-d6) δ ppm 4.08 (s, 3 H), 4.14 (s, 3 H), 7.49 (s, 1 H), 7.97 (s, 1 H), 8.36 (s, 2 H), 8.69 (s, 1 H), | | | | | |
| | 8.86 (d, J = 0.98 Hz, 1 H), 11.36 (s, 1 H). MS (M + 1): 419.0 | | | | | |
| 1L | —CH₃ | CH | CH | —SO₂CH₃ | CH | 5-methyl- |
| | | | | | | pyrazin-2-yl |
| | ¹H NMR (400 MHz, CHLOROFORM-d) δ ppm 2.54 (s, 3 H), 3.08 (s, 3 H), 4.24 (s, 3 H), 7.14-7.23 (m, 2 H), 7.78-7.98 | | | | | |
| | (m, 4 H), 8.12 (d, J = 18.96 Hz, 2 H), 8.61 (s, 1 H), 9.54 (s, 1 H). MS (M + 1): 438.1 | | | | | |
| 1M | —CH₃ | CH | CH | —SO₂CH₃ | CH | 5-methyl- |
| | | | | | | pyridin-2-yl |
| | ¹H NMR (400 MHz, CHLOROFORM-d) δ ppm 2.30 (s, 3 H), 3.07 (s, 3 H), 4.23 (s, 3 H), 6.44 (d, 1 H), 7.11-7.23 (m, 2 | | | | | |
| | H), 7.57 (d, 1 H), 7.79 (s, 1 H), 7.90 (s, 1 H), 7.92 (s, 1 H), 8.09 (d, 2 H), 8.22 (d, 1 H), 8.64 (s, 1 H). MS (M + 1): 437.0 | | | | | |
| 1N | —CH₃ | CH | CH | —SO₂CH₃ | CH | 1-methyl-1H- |
| | | | | | | pyrazol-3-yl |
| | ¹H NMR (400 MHz, CHLOROFORM-d) δ ppm 3.06 (s, 3 H), 3.76 (s, 3 H), 4.21 (s, 3 H), 5.54 (d, J = 1.76 Hz, 1 H), 6.79 (d, | | | | | |
| | J = 1.56 Hz, 1 H), 7.16 (s, 1 H), 7.17 (s, 1 H), 7.27 (d, J = 1.95 Hz, 1 H), 7.79 (s, 1 H), 7.89 (s, 1 H), 7.91 (s, 1 H), 8.02 (s, 1 | | | | | |
| | H), 8.85 (s, 1 H). MS (M + 1): 426.0 | | | | | |
| 1O | —CH₂CH₃ | CH | N | —C(O)N(CH₃)₂ | N | 5-methyl- |
| | | | | | | pyridin-2-yl |
| | ¹H NMR (400 MHz, CHLOROFORM-d) δ ppm 1.63 (t, J = 7.2 Hz, 3 H), 2.30 (s, 3 H), 3.14 (s, 3 H), 3.19 (s, 3 H), 4.48 (q, | | | | | |
| | J = 7.2 Hz, 2 H), 7.44 (s, 1 H), 7.55-7.57 (m, 1 H), 7.79 (s, 1 H), 8.11 (s, 1 H), 8.20 (s, 1 H), 8.26 (d, J = 8.4 Hz, 1 H), 8.44 | | | | | |
| | (s, 1 H), 8.49 (s, 1 H), 8.67 (s, 1 H). MS (M + 1): 446.1 | | | | | |
| 1P | —CH₂CH₃ | CH | N | —C(O)N(CH₃)₂ | N | 5-methyl- |
| | | | | | | pyrazin-2-yl |
| | ¹H NMR (400 MHz, CHLOROFORM-d) δ ppm 1.63 (t, J = 7.2 Hz, 3 H), 2.55 (s, 3 H), 3.15 (s, 3 H), 3.17 (s, 3 H), 4.49 (q, | | | | | |
| | J = 7.2 Hz, 2 H), 7.45 (s, 1 H), 7.81 (s, 1 H), 8.14 (s, 1 H), 8.20 (s, 1 H), 8.45 (s, 1 H), 8.50 (s, 1 H), 8.54 (s, 1 H), 9.57 | | | | | |
| | (s, 1 H). MS (M + 1): 447.1 | | | | | |
| 1Q | —CH₂CH₃ | CH | CH | —C(O)N(CH₃)₂ | CH | pyridin-2-yl |
| | ¹H NMR (400 MHz, CHLOROFORM-d) δ ppm 1.65 (t, J = 7.2 Hz, 3 H), 3.08 (s, 3 H), 4.49 (q, J = 7.2 Hz, 2 H), 7.06-7.09 (m, | | | | | |
| | 1 H), 7.19-7.22 (m, 3 H), 7.73-7.78 (m, 1 H), 7.85 (s, 1 H), 7.93 (d, J = 8.8 Hz, 2 H), 8.13 (s, 1 H), 8.30-8.31 (m, 1 H), | | | | | |
| | 8.33-8.36 (m, 1 H), 8.66 (s, 1 H). MS (M + 1): 437.0 | | | | | |
| 1R | —CH₂CH₃ | CH | CH | —C(O)N(CH₃)₂ | CH | 5-methyl- |
| | | | | | | pyridin-2-yl |
| | ¹H NMR (400 MHz, CHLOROFORM-d) δ ppm 1.64 (t, J = 7.6 Hz, 3 H), 2.30 (s. 3 H), 3.07 (s, 3 H), 4.48 (q, J = 7.2 Hz, 2 H), | | | | | |
| | 7.16-7.21 (m, 3 H), 7.54-7.57 (m, 1 H), 7.84 (s, 1 H), 7.92 (d, J = 9.2 Hz, 2 H), 8.10 (s, 1 H), 8.13 (s, 1 H), 8.23 (d, J = 8.4 Hz, | | | | | |
| | 1 H), 8.62 (s, 1 H). MS (M + 1): 451.0 | | | | | |
| 1S | —CH₃ | CH | CH | —SO₂CH₂CH₃ | CH | pyridin-2-yl |
| | ¹H NMR (400 MHz, CHLOROFORM-d) δ ppm 1.30 (t, J = 7.43 Hz, 3 H), 3.13 (q, J = 7.36 Hz, 2 H), 4.23 (s, 3 H), 7.07 (dd, | | | | | |
| | J = 6.55, 4.98 Hz, 1 H), 7.18 (d, J = 8.79 Hz, 2 H) 7.21-7.28 (m, 1 H), 7.71-7.80 (m, 2 H), 7.88 (d, J = 8.79 Hz, 2 H), 8.11 | | | | | |
| | (s, 1 H), 8.31 (br. s., 1 H), 8.34 (d, J = 8.40 Hz, 1 H), 8.64 (br. s., 1 H). MS (M + 1): 437.9. | | | | | |
| 1T | —CH₃ | CH | CH | —SO₂CH₂CH₃ | CH | 5-methyl- |
| | | | | | | pyrazin-2-yl |
| | ¹H NMR (400 MHz, CHLOROFORM-d) δ ppm 1.30 (m, 3 H), 2.54 (s, 3 H), 3.12 (m, 2 H), 4.23 (s, 3 H), 7.2 (m, 3 H), 7.79 | | | | | |
| | (s, 1 H), 7.86 (m, 2 H), 8.10 (m, 2 H), 8.56 (s, 1 H), 9.54 (s, 1 H). MS (M + 1): 451.8. | | | | | |
| 1U | —CH₃ | CH | CH | 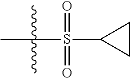 | CH | 5-methyl- pyrazin-2-yl |
| | ¹H NMR (400 MHz, CHLOROFORM-d) δ ppm 0.97-1.12 (m, 2 H), 1.30-1.44 (m, 2 H), 2.43-2.52 (m, 1 H), 2.55 (s, | | | | | |
| | 3 H), 4.24 (s, 3 H), 7.21 (dd, J = 20.03, 9.09 Hz, 3 H), 7.82 (s, 1 H), 7.88 (d, J = 8.60 Hz, 2 H), 8.08 (s, 1 H), 8.17 (s, 1 H), | | | | | |
| | 8.49 (br. s, 1 H), 9.54 (s, 1 H). MS (M + 1): 463.9. | | | | | |
| 1V | —CH₃ | CH | CH | 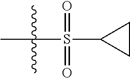 | CH | pyridin-2-yl |
| | ¹H NMR (400 MHz, CHLOROFORM-d) δ ppm 1.06 (br. s, 2 H), 1.29-1.45 (m, 2 H), 2.48 (br. s, 1 H), 4.23 (br. s, 3 H), 7.06 (s, 1 H), | | | | | |
| | 7.12-7.30 (m, 3 H), 7.68-7.82 (m, 2 H), 7.83-7.93 (m, 2 H), 8.10 (s, 1 H), 8.19-8.40 (m, 2 H), 8.63 (s, 1 H). MS (M + 1): 448.9. | | | | | |
| 1W | —CH₂CH₃ | CH | CH | 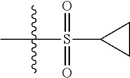 | CH | 5-methyl- pyrazin-2-yl |
| | ¹H NMR (400 MHz, CHLOROFORM-d) δ ppm 1.06 (dd, J = 7.91, 2.05 Hz, 2 H), 1.36 (dd, J = 4.79, 1.86 Hz, 2 H), 1.65 | | | | | |
| | (t, J = 7.33 Hz, 3 H), 2.43-2.53 (m, 1 H), 2.55 (s, 3 H), 4.50 (q, J = 7.43 Hz, 2 H), 7.17-7.20 (m, 1 H), 7.20-7.23 (m, 2 | | | | | |
| | H), 7.85-7.88 (m, 2 H), 7.88-7.91 (m, 1 H), 8.10 (s, 1 H), 8.15 (d, J = 0.98 Hz, 1 H), 8.46 (s, 1 H), 9.54 (d, J = 1.37 Hz, 1 | | | | | |
| | H). MS (M + 1): 477.9. | | | | | |
| 1X | —CH₂CH₃ | CH | CH | 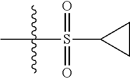 | CH | pyridin-2-yl |
| | ¹H NMR (400 MHz, CHLOROFORM-d) δ ppm 1.06 (dd, J = 7.82, 1.95 Hz, 2 H), 1.34 (br. s, 2 H), 1.64 (t, J = 7.33 Hz, | | | | | |
| | 3H), 2.48 (s, 1 H), 4.49 (q, J = 7.30 Hz, 2 H), 7.07 (d, J = 7.04 Hz, 1 H), 7.16-7.23 (m, 2 H), 7.25 (s, 1 H), 7.70-7.80 (m, | | | | | |
| | 1 H), 7.83-7.91 (m, 3 H), 8.11 (s, 1 H), 8.26-8.39 (m, 2 H), 8.62 (br. s, 1 H). MS (M + 1): 462.9. | | | | | |
| 1Y | —CH₃ | N | CH | —C(O)N(CH₃)₂ | N | 5-methyl- |
| | | | | | | pyrazin-2-yl |
| | ¹H NMR (400 MHz, CHLOROFORM-d) δ ppm 2.55 (s, 3 H), 3.00 (s, 3 H), 3.15 (s, 3 H), 4.26 (s, 3 H), 7.86 (s, 1 H), 8.13 | | | | | |
| | (d, J = 14.64 Hz, 2 H), 8.49 (s, 1 H), 8.58 (s, 2 H), 9.54 (d, J = 1.37 Hz, 1 H). MS (M + 1): 433.4. | | | | | |
| 1Z | —CH₃ | CH | N | —C(O)N(CH₃)₂ | N | 5-methyl- |
| | | | | | | pyridin-2-yl |
| | ¹H NMR (400 MHz, CHLOROFORM-d) δ ppm 2.33 (s, 3 H), 3.00 (s, 3 H), 3.15 (s, 3 H), 4.25 (s, 3 H), 7.30 (s, 1 H), 7.65 | | | | | |
| | (d, J = 8.59 Hz, 1 H), 7.84 (s, 1 H), 8.10 (br. s, 1 H), 8.20 (s, 1 H), 8.34 (d, J = 8.39 Hz, 1 H), 8.58 (s, 2 H), 9.22 (br. s, 1 H). | | | | | |
| | MS (M + 1): 432.4. | | | | | |
| 1AA | —CH₃ | CH | N | —C(O)N(CH₃)₂ | N | 5-chloro- |
| | | | | | | pyridin-2-yl |
| | ¹H NMR (400 MHz, CHLOROFORM-d) δ ppm 3.01 (s, 3 H), 3.17 (s, 3 H), 4.27 (s, 3 H), 7.25 (s, 1 H), 7.74 (dd, J = 8.98, | | | | | |
| | 2.54 Hz, 1 H), 7.87 (s, 1 H), 8.14 (s, 1 H), 8.28 (br. s, 1 H), 8.36 (d, J = 8.79 Hz, 1 H), 8.59 (s, 2 H), 8.72 (br. s, 1 H). MS | | | | | |
| | (M + 1): 452.0. | | | | | |
| 1AB | —CH₃ | CH | N | —C(O)N(CH₃)₂ | N | 5- |
| | | | | | | (trifluoromethyl)- |
| | | | | | | pyridin-2-yl |
| | ¹H NMR (400 MHz, CHLOROFORM-d) δ ppm 3.01 (s, 3 H), 3.17 (s, 3 H), 4.28 (s, 3 H), 7.25 (s, 1 H), 7.88 (s, 1 H), 7.99 | | | | | |
| | (dd, J = 8.69, 2.05 Hz, 1 H), 8.15 (s, 1 H), 8.50 (d, J = 8.59 Hz, 1 H), 8.60 (br. s, 3 H) 8.83 (s, 1 H). MS (M + 1): 486.0. | | | | | |
| 1AC | —CH₃ | CH | CF | —C(O)N(CH₃)₂ | N | 5-methyl- |
| | | | | | | pyrazin-2-yl |
| | ¹H NMR (400 MHz, CHLOROFORM-d) δ ppm 2.54 (s, 3 H), 2.99 (s, 3 H), 3.16 (s, 3 H), 4.26 (s, 3 H), 7.13 (dd, J = 9.76, | | | | | |
| | 2.34 Hz, 1 H), 7.28 (s, 1 H), 7.85 (s, 1 H), 8.15 (d, J = 8.98 Hz, 2 H), 8.32 (d, J = 1.76 Hz, 1 H), 8.59 (s, 1 H), 9.55 (d, | | | | | |
| | J = 1.37 Hz, 1 H). MS (M + 1): 450.5. | | | | | |
| 1AD | —CH₂CH₃ | CH | CH | —SO₂CH₂CH₃ | CH | 5-methyl- |
| | | | | | | pyridin-2-yl |
| | ¹H NMR (400 MHz, CHLOROFORM-d) δ ppm 1.30 (t, J = 7.6 Hz, 3 H), 1.63 (t, J = 7.2 Hz, 3 H), 2.28 (s, 3 H), 3.12 (q, J = 7.6 | | | | | |
| | Hz, 2 H), 4.47 (q, J = 7.6 Hz, 2 H), 7.16-7.21 (m, 3 H), 7.54-7.56 (m, 1 H), 7.82 (s, 1 H), 7.87 (d, J = 8.8 Hz, 2 H), 8.09- | | | | | |
| | 8.11 (m, 2 H), 8.22 (d, J = 8.4 Hz, 1 H), 8.66 (s, 1 H). MS (M + 1): 465.0. | | | | | |
| 1AE | —CH₂CH₃ | CH | CF | —C(O)N(CH₃)₂ | N | 5-methyl- |
| | | | | | | pyrazin-2-yl |
| | ¹H NMR (400 MHz, CHLOROFORM-d) δ ppm 1.61-1.65 (m, 3 H), 2.51 (s, 3 H), 2.96 (s, 3 H), 3.12 (s, 3 H), 4.45-4.51 (m, 2 H), | | | | | |
| | 7.10 (dd, J = 10, 2.4 Hz, 1 H), 7.24 (s, 1 H), 7.87 (s, 1 H), 8.08-8.17 (m, 2 H), 8.29 (s, 1 H), 8.69 (s, 1 H), 9.52 (s, 1 H). MS | | | | | |
| | (M + 1): 464.0. | | | | | |
| 1AF | —CH₂CH₃ | N | CH | —C(O)N(CH₃)₂ | N | 5-methoxy- |
| | | | | | | pyrazin-2-yl |
| | ¹H NMR (400 MHz, CHLOROFORM-d) δ ppm 1.66 (t, 3 H), 3.17 (s, 3 H), 3.20 (s, 3 H), 4.00 (s, 3 H), 4.51 (q, J = 7.3 Hz, 2 | | | | | |
| | H), 7.47 (d, J = 1.2 Hz, 1 H), 7.82 (d, J = 0.8 Hz, 1 H), 7.97 (d, J = 1.6 Hz, 1 H), 8.20 (t, J = 1.1 Hz, 1 H), 8.43-8.46 (m, 1 H), | | | | | |
| | 8.47 (d, J = 1.4 Hz, 1 H), 8.52 (d, J = 1.4 Hz, 1 H), 9.21 (d, J = 1.4 Hz, 1 H). MS (M + 1): 463.5 | | | | | |
| 1AG | —CH₃ | CH | CH | C(O)N(CH₃)₂ | CH | 5-methyl- |
| | | | | | | pyrazin-2-yl |
| | ¹H NMR (400 MHz, CHLOROFORM-d) δ ppm 2.38 (s, 3 H), 3.04 (m, 6 H), 4.22 (s, 3 H), 7.09 (d, J = 8.58 Hz, 2 H), 7.45 (d, | | | | | |
| | J = 8.58 Hz, 2 H), 7.80 (s, 1 H), 7.85 (s, 1 H), 7.90 (s, 1 H), 8.03 (s, 1 H), 8.14 (s, 1 H), 8.48 (s, 1 H). MS (M + 1): 430.9 | | | | | |
| 1AH | —CH₃ | N | CH | 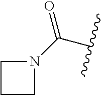 | N | 2-methyl-2H- 1,2,3-triazol-4-yl |
| | ¹H NMR (400 MHz, CHLOROFORM-d) δ ppm 2.31-2.42 (m, 2 H), 4.09 (s, 3 H), 4.22 (s, 3 H), 4.25 (t, 2 H), 4.67 (t, J = 7.80 | | | | | |
| | Hz, 2 H), 6.89 (d, J = 6.63 Hz, 1 H), 7.48 (d, J = 1.17 Hz, 1 H), 7.72 (s, 1 H), 8.16 (s, 1 H), 8.39 (d, J = 1.37 Hz, 1 H), 8.83 (d, | | | | | |
| | J = 1.37 Hz, 1 H), 9.51 (s, 1 H). MS (M + 1): 434.4 | | | | | |
| 1AI | —CH₃ | CH | CH | 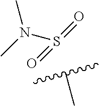 | CH | 5-methyl- pyrazin-2-yl |
| | ¹H NMR (400 MHz, CHLOROFORM-d) δ ppm 2.39 (s, 3 H), 2.74 (s, 6 H), 4.24 (s, 3 H), 7.15-7.18 (m, 1 H), 7.17-7.20 (m, | | | | | |
| | 1 H), 7.23 (d, J = 1.17 Hz, 1 H), 7.74-7.77 (m, 1 H), 7.77- 7.79 (m, 1 H), 7.80 (s, 1 H), 8.09 (s, 1 H), 8.15 (d, J = 0.98 Hz, 1 H), | | | | | |
| | 8.47 (s, 1 H), 9.55 (d, J = 1.37 Hz, 1 H). MS (M + 1): 466.9. | | | | | |
| 1AJ | —CH₃ | CH | CH | 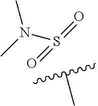 | CH | 2-methyl-triazol- 4-yl |
| | ¹H NMR (400 MHz, CHLOROFORM-d) δ ppm 2.73 (s, 6 H), 4.11 (s, 3 H), 4.23 (s, 3 H), 7.08-7.23 (m, 3 H), 7.78 (d, J = 9.17 | | | | | |
| | Hz, 3 H), 8.06 (d, J = 24.78 Hz, 2 H), 8.55 (br. s., 1 H). MS (M + 1): 455.9. | | | | | |
| 1AK | —CH₃ | CH | CH | 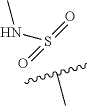 | CH | 5-methyl- pyrazin-2-yl |
| | ¹H NMR (400 MHz, CHLOROFORM-d) δ ppm 2.16 (s, 3 H), 2.70 (d, J = 3.51 Hz, 3 H), 4.24 (s, 3 H), 4.49 (br. s., 1 H), 7.15 | | | | | |
| | (s, 1 H), 7.17 (s, 1 H), 7.20 (d, J = 1.17 Hz, 1 H), 7.82 (s, 1 H), 7.83-7.84 (m, 1 H), 7.84-7.86 (m, 1 H) 8.07-8.11 (m, 1 H) | | | | | |
| | 8.15 (d, J = 0.98 Hz, 1 H) 8.56 (s, 1 H), 9.54 (d, J = 1.56 Hz, 1 H). MS (M + 1): 453.4. | | | | | |
| 1AL | —CH₃ | CH | N | 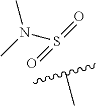 | CH | 5-methyl- pyrazin-2-yl |
| | ¹H NMR (400 MHz, CHLOROFORM-d) δ ppm 2.55 (s, 3 H), 2.75 (s, 6 H), 4.24 (s, 3 H), 7.16 (d, 1 H, J = 8.8), 7.45 (d, 1 H, J = | | | | | |
| | 2.1), 7.77 (s, 1 H), 8.09 (dd, 1 H, J = 8.4, 1.8), 8.15 (s, 1 H), 8.19 (s, 1 H), 8.53 (d, 1 H, J = 2.0), 8.60 (s, 1 H), 9.60 (s, 1 H). | | | | | |
| | MS (M + 1): 468.2. | | | | | |
| 1AM | —CH₃ | CH | N | 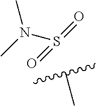 | N | 1-methyl- pyrazol-3-yl |
| | ¹H NMR (400 MHz, CHLOROFORM-d) δ ppm 2.74 (s, 6 H), 3.79 (s, 3 H), 4.21 (s, 3 H), 6.81 (s, 1 H), 7.12 (d, 1 H, J = 8.4), | | | | | |
| | 7.28 (s, 1 H), 7.40 (s, 1 H), 7.75 (s, 1 H), 8.06 (dd, 1 H, J = 8.8, 1.8), 8.12 (s, 1 H), 8.53 (d, 1 H, J = 2.4), 8.70 (s, 1 H). MS | | | | | |
| | (M + 1): 456.1. | | | | | |
| 1AN | —CH₂CH₃ | N | CH | 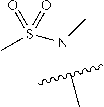 | N | 5-methyl-pyridin- 2-yl |
| | ¹H NMR (400 MHz, DMSO-d6) δ ppm 1.52 (t, J = 7.41 Hz, 3 H), 2.29 (s, 3 H), 3.16 (s, 3 H), 3.30 (s, 3 H), 4.48 (q, J = 7.22 Hz, 2 | | | | | |
| | H), 7.39 (d, J = 0.98 Hz, 1 H), 7.66 (dd, J = 8.49, 2.05 Hz, 1 H), 8.07 (d, J = 8.58 Hz, 1 H), 8.23 (d, J = 2.54 Hz, 1 H), 8.25 (d, | | | | | |
| | J = 1.37 Hz, 1 H), 8.32-8.36 (m, 1 H), 8.42 (d, J = 0.78 Hz, 1 H), 8.55 (d, J = 1.37 Hz, 1 H) 10.79 (s, 1 H). MS (M + 1): 482.4. | | | | | |
| 1AO | —CH₃ | CH | N | 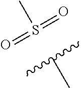 | CH | 1-methyl- pyrazol-3-yl |
| | ¹H NMR (400 MHz, CHLOROFORM-d) δ ppm 3.24 (s, 3 H), 3.82 (s, 3 H), 4.24 (s, 3 H), 6.79 (s, 1 H), 7.23 (s, 1 H), 7.29 (s, | | | | | |
| | 1 H), 7.48 (dd, 1 H, J = 8.4, 2.8), 7.82 (s, 1 H), 8.05-8.07 (m, 2 H), 8.53 (d, 1 H, J = 2.4), 8.62 (s, 1 H). MS (M + 1): 427.7. | | | | | |
| 1AP | —CH₃ | CH | N | 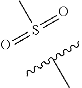 | CH | 5-methyl-pyridin- 2yl |
| | ¹H NMR (400 MHz, CHLOROFORM-d) δ ppm 2.31 (s, 3 H), 3.24 (s, 3 H), 4.25 (s, 3 H), 7.26 (s, 1 H), 7.46-7.53 (m, 2 H), | | | | | |
| | 7.56 (dd, 1 H, J = 8.4, 2.0), 7.81 (s, 1 H), 8.06 (d, 1 H, J = 8.4), 8.12-8.14 (m, 2 H), 8.23 (d, 1 H, J = 8.8), 8.53 (d, 1 H, J = 2.4), | | | | | |
| | 8.63 (s, 1 H). MS (M + 1): 438.7. | | | | | |
| 1AQ | —CH₃ | CH | N | 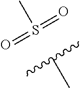 | CH | 5-methyl- pyrazin-2-yl |
| | ¹H NMR (400 MHz, CHLOROFORM-d) δ ppm 2.55 (s, 3 H), 3.24 (s, 3 H), 4.25 (s, 3 H), 7.26 (s, 1 H), 7.49 (dd, 1 H, J = 8.8, | | | | | |
| | 2.8), 7.84 (s, 1 H), 8.06 (d, 1 H, J = 8.4), 8.13 (s, 1 H), 8.15 (s, 1 H), 8.50 (s, 1 H), 8.53 (d, 1 H, J = 2.0), 9.54 (s, 1 H). MS | | | | | |
| | (M + 1): 439.7. | | | | | |
| 1AR | —CH₃ | C—CF3 | CH | 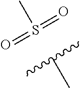 | CH | 5-methyl-pyridin- 2-yl |
| | ¹H NMR (400 MHz, CHLOROFORM-d) δ ppm 2.31 (s, 3 H), 3.11 (s, 3 H), 4.24 (s, 3 H), 7.12 (d, J = 8.60 Hz, 1 H), 7.30 (s, 1 | | | | | |
| | H), 7.58 (d, 1 H), 7.82 (s, 1 H), 7.99 (d, 1 H), 8.16 (d, 2 H), 8.24 (d, 1 H), 8.31 (s, 1 H), 8.58 (br. s., 1 H). MS (M + 1): 504.8. | | | | | |
| 1AS | —CH₃ | CH | N | 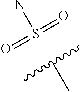 | CH | 1-methyl- pyrazol-3-yl |
| | ¹H NMR (400 MHz, DMSO-d6) δ ppm 2.44 (s, 3 H), 4.17 (s, 3 H), 7.12 (s, 1 H), 7.24 (d, 2 H, J = 8.8), 7.32 (s, 2 H), 7.82 (d, 2 | | | | | |
| | H, J = 8.8), 8.31 (s, 1 H), 8.37 (s, 1 H), 8.72 (s, 1 H), 9.21 (s, 1 H), 11.06 (s, 1 H). MS (M + 1): 439.1. | | | | | |
| 1AT | —CH₂CH₃ | N | CH | 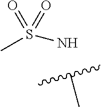 | N | 5-methyl-pyridin- 2-yl |
| | 1H NMR (400 MHz, DMSO-d6) ppm 1.47 (t, J = 7.33 Hz, 3 H), 2.25 (s, 3 H), 3.24 (br. s., 3 H), 4.33-4.55 (m, 2 H), 7.23 (d, | | | | | |
| | J = 1.17 Hz, 1 H), 7.63 (s, 1 H), 7.92 (d, J = 1.37 Hz, 1 H), 7.99-8.07 (m, 1 H), 8.19 (d, J = 2.35 Hz, 1 H), 8.25 (s, 1 H), 8.34- | | | | | |
| | 8.44 (m, 2 H), 10.73 (s, 1 H), 10.81 (s, 1 H). MS (M + 1): 468.4. | | | | | |
| 1AU | —CH₃ | CH | N | 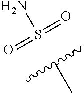 | CH | 1-methyl- pyrazol-3-yl |

¹H NMR (400 MHz, DMSO-d6) δ ppm 3.73 (s, 3 H), 4.15 (s, 3 H), 6.59 (s, 1 H), 7.09 (s, 1 H), 7.22 (d, 2 H, J = 8.8), 7.31 (s, 2
H), 7.65 (s, 1 H), 7.82 (d, 2 H, J = 8.8), 8.21 (s, 1 H), 8.33 (s, 1 H), 10.85 (s, 1 H). MS (M + 1): 427.1.

### Example 1AV

### Preparation of 4-[(1,1-dioxido-2,3-dihydro-1-benzothien-5-yl)oxy]-2-methyl-N-(5-methylpyridin-2-yl)-2H-indazole-6-carboxamide (1AV)

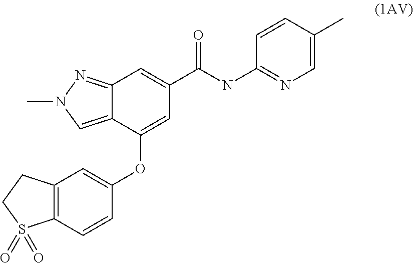

The title compound was prepared by a method analogous to that described for Example (1A), using ethyl 4-[(1,1-dioxido-2,3-dihydro-1-benzothien-5-yl)oxy]-2-methyl-2H-indazole-6-carboxylate (I-1f-29).

¹H NMR (400 MHz, CHLOROFORM-d) δ ppm 2.31 (s, 3H), 3.31 (t, J=6.93 Hz, 2H), 3.50 (t, J=6.93 Hz, 2H), 4.23 (s, 3H), 6.93 (d, J=1.56 Hz, 1 H), 7.10-7.21 (m, 2H), 7.56 (dd, J=8.49, 2.05 Hz, 1H), 7.71 (d, J=8.59 Hz, 1H), 7.76 (s, 1H), 8.01-8.16 (m, 2H), 8.23 (d, J=8.39 Hz, 1H), 8.50 (s, 1 H). MS (M+1): 449.3.

### Example 1AW

### Preparation of 4-[(1,1-dioxido-2,3-dihydro-1-benzothien-5-yl)oxy]-2-ethyl-N-(5-methylpyridin-2-yl)-2H-indazole-6-carboxamide (1AW)

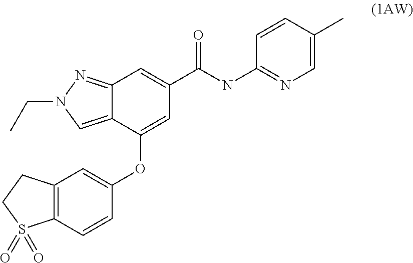

The title compound was prepared by a method analogous to that described for Example (1A), using ethyl 4-[(1,1-dioxido-2,3-dihydro-1-benzothien-5-yl)oxy]-2-ethyl-2H-indazole-6-carboxylate (I-1f-30).

¹H NMR (400 MHz, CHLOROFORM-d) δ ppm 1.62 (t, J=7.42 Hz, 3 H), 2.26 (s, 3H), 3.29 (t, J=6.93 Hz, 2H), 3.48 (t, J=6.93 Hz, 2H), 4.46 (q, J=7.35 Hz, 2H), 6.92 (d, J=1.76 Hz, 1H), 7.05-7.20 (m, 2H), 7.53 (dd, J=8.49, 2.05 Hz, 1H), 7.68 (d, J=8.39 Hz, 1H), 7.80 (s, 1H), 7.96-8.13 (m, 2H), 8.21 (d, J=8.39 Hz, 1H), 8.78 (s, 1H). MS (M+1): 463.3.

### Example 1AX

### Preparation of 2-ethyl-N-(5-methoxypyrazin-2-yl)-4-[4-(methylsulfonyl)phenoxy]-2H-indazole-6-carboxamide (1AX)

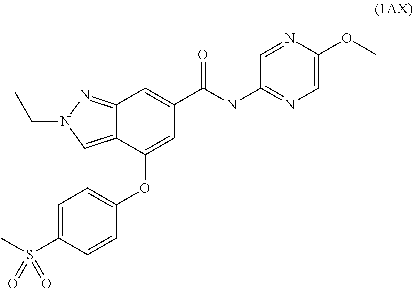

To a solution of 5-methoxypyrazin-2-amine (167 mg, 1.34 mmol) in dry dichloroethane (2.0 mL) was added dimethylaluminum chloride (2.67 mL, 2.67 mmol) slowly at 0° C. under nitrogen. After addition, the mixture was stirred for 1 hour at room temperature and then added methyl 2-ethyl-4-(4-(methylsulfonyl)phenoxy)-2H-indazole-6-carboxylate (I-1f-7, 100.0 mg, 0.267 mmol). Then the mixture was heated to 60-70° C. overnight. The reaction mixture was cooled to room temperature, poured into potassium sodium tartrate (aq., 10 mL), and extracted with ethyl acetate (10 mL 3 times). The combined organic layers were dried and concentrated under reduced pressure and purified by preparative HPLC to afford the title compound (1AX, 20.0 mg, yield: 16%) as a white solid.

¹H NMR (400 MHz, DMSO-d6): δ 1.46-1.49 (t, 3H), 3.15 (s, 3H), 3.88 (s, 3H), 4.43-4.49 (q, 2H), 7.20 (s, 1H), 7.17-7.31 (d, 2H), 7.91-7.93 (d, 2H), 8.15 (s, 1H), 8.32 (s, 1H), 8.44 (s, 1H), 8.88 (s, 1H), 10.97 (s, 1H). MS (M+1): 468.2.

The compounds listed in Table 2 below were prepared using procedures analogous to those for the preparation of Compound (1AX) above using the appropriate starting materials and intermediates described above.

TABLE 2

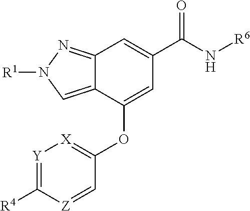

| Example No. | R¹ | X | Y | R⁴ | Z | R⁶ |
|---|---|---|---|---|---|---|
| 1AY | —CH₃ | N | CH | 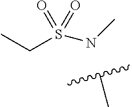 | N | 5-methyl-pyridin- 2-yl |
| | MS (M + 1): 482.13. RT: 2.34 min. Column: Waters Atlantis C18 4.6 × 50 mm, 5 μm. Modifier: TFA 0.05%. Gradient: 95% | | | | | |
| | H2O/5% MeCN linear to 5% H2O/95% MeCN over 4.0 min and held to 5.0 min. Flow rate: 2.0 mL/min | | | | | |
| 1AZ | —CH₂CH₃ | CH | CF | 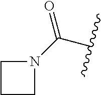 | N | 5-methyl- pyrazin-2-yl |
| | ¹H NMR (400 MHz, CHLOROFORM-d) δ ppm 1.68 (t, 3 H), 2.32-2.42 (m, 2 H), 2.58 (s, 3 H), 4.27 (t, J = 7.8 Hz, 2 H), | | | | | |
| | 4.36-4.42 (m, 2 H), 4.52 (q, J = 7.3 Hz, 2 H), 7.14 (dd, J = 10.5, 2.3 Hz, 1 H), 7.29 (d, J = 1.2 Hz, 1 H), 7.87 (d, J = 0.8 | | | | | |
| | Hz, 1 H), 8.16 (t, J = 1.1 Hz, 1 H), 8.17 (d, J = 1.0 Hz, 1 H), 8.30 (dd, 1 H), 8.55 (s, 1 H), 9.57 (d, J = 1.4 Hz, 1 H). MS | | | | | |
| | (M + 1): 476.2 | | | | | |
| 1BA | —CH₂CH₃ | CH | CF | 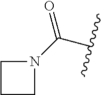 | N | 1-methyl-1H- pyrazol-3-yl |
| | ¹H NMR (400 MHz, CHLOROFORM-d) δ ppm 1.67 (t, 3 H), 2.36 (quin, J = 7.8 Hz, 2 H), 3.84 (s, 3 H), 4.27 (t, J = 7.8 Hz, 2 | | | | | |
| | H), 4.36-4.42 (m, 2 H), 4.51 (q, J = 7.4 Hz, 2 H), 6.82 (d, J = 2.1 Hz, 1 H), 7.13 (dd, J = 10.5, 2.3 Hz, 1 H), 7.26 (d, J = 1.2 | | | | | |
| | Hz, 1 H), 7.31 (d, J = 2.3 Hz, 1 H), 7.85 (d, 1 H), 8.08 (t, 1 H), 8.29 (dd, 1 H), 8.46 (s, 1 H). MS (M + 1): 464.5 | | | | | |
| 1BB | —CH₂CH₃ | CH | CF | —C(O)N(CH₃)₂ | N | pyrazin-2-yl |
| | 1H NMR (400 MHz, CHLOROFORM-d) δ ppm 1.69 (t, 3 H), 3.01 (s, 3 H), 3.18 (s, 3 H), 4.54 (q, J = 7.3 Hz, 2 H), 7.16 (dd, | | | | | |
| | J = 9.8, 2.3 Hz, 1 H), 7.29 (d, J = 1.2 Hz, 1 H), 7.91 (s, 1 H), 8.17 (t, 1 H), 8.32 (dd, J = 2.5, 1.6 Hz, 1 H), 8.35 (dd, J = 2.3, | | | | | |
| | 0.8 Hz, 1 H), 8.42 (d, J = 2.5 Hz, 1 H), 8.57 (s, 1 H), 9.70 (d, J = 1.6 Hz, 1 H). MS (M + 1): 450.2 | | | | | |
| 1BC | —CH₃ | CH | CF | 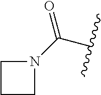 | N | 5-methyl- pyrazin-2-yl |
| | ¹H NMR (400 MHz, CHLOROFORM-d) δ ppm 2.32-2.41 (m, 2 H), 2.58 (s, 3 H), 4.24-4.30 (m, 5 H), 4.39 (t, J = 7.6 Hz, 2 | | | | | |
| | H), 7.13 (dd, J = 10.5, 2.3 Hz, 1 H), 7.30 (d, J = 1.2 Hz, 1 H), 7.83 (s, 1 H), 8.14 (t, J = 1.1 Hz, 1 H), 8.17-8.19 (m, 1 H), | | | | | |
| | 8.28-8.31 (m, 1 H), 8.47 (s, 1 H), 9.57 (d, J = 1.6 Hz, 1 H). MS (M + 1): 462.2 | | | | | |
| 1BD | —CH₂CH₃ | CH | N | —C(O)N(CH₃)₂ | N | 5-methyl- |
| | | | | | | pyridin-2-yl |
| | ¹H NMR (400 MHz, CHLOROFORM-d) δ ppm 1.67 (t, 3 H), 2.32 (s, 3 H), 3.02 (s, 3 H), 3.17 (s, 3 H), 4.52 (q, J = 7.3 Hz, 2 | | | | | |
| | H), 7.25 (d, J = 1.2 Hz, 1 H), 7.58 (dd, J = 8.6, 2.3 Hz, 1 H), 7.91 (s, 1 H), 8.13 (d, J = 2.3 Hz, 1 H), 8.16 (t, 1 H), 8.25 (d, | | | | | |
| | J = 8.4 Hz, 1 H), 8.60 (s, 2 H), 8.71 (s, 1 H). MS (M + 1): 446.5 | | | | | |
| 1BE | —CH₃ | CH | CF | —C(O)N(CH₃)₂ | N | 5-methoxy- |
| | | | | | | pyrazin-2-yl |
| | ¹H NMR (400 MHz, CHLOROFORM-d) δ ppm 3.01 (s, 3 H), 3.18 (s, 3 H), 4.01 (s, 3 H), 4.28 (s, 3 H), 7.14 (dd, J = 9.9, 2.2 | | | | | |
| | Hz, 1 H), 7.29 (d, J = 1.0 Hz, 1 H), 7.85 (s, 1 H), 7.98 (d, J = 1.6 Hz, 1 H), 8.12 (s, 1 H), 8.34 (d, J = 2.3 Hz, 1 H), 8.39 (s, 1 H), | | | | | |
| | 9.19 (d, J = 1.4 Hz, 1 H). MS (M + 1): 466.5 | | | | | |
| 1BG | —CH₂CH₃ | CH | CH | 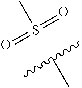 | CH | 5-ethoxy- pyrazin-2-yl |
| | ¹H NMR (400 MHz, METHANOL-d4) δ ppm 1.37-1.43 (m, 3 H), 1.54-1.63 (m, 3 H), 2.78-2.84 (m, 2 H), 3.12 (s, 3 H), | | | | | |
| | 4.35-4.45(m, 2 H), 4.45-4.55 (m, 2 H), 7.24 (s, 1 H), 7.30 (d, 2 H), 7.95-8.03 (m, 3 H), 8.20 (d, 2 H), 8.92 (s, 1 H). MS | | | | | |
| | (M + 1): 482.3 | | | | | |
| 1BH | —CH₂CH₃ | CH | CH | 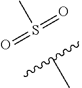 | CH | 5-ethyl- pyrazin-2-yl |
| | ¹H NMR (400 MHz, DMSO-d6) δ ppm 1.24-1.32 (t, 3 H), 1.62-1.66 (t, 3 H), 2.78-2.84 (q, 2 H), 3.08 (s, 3 H), 4.46-4.52 (q, | | | | | |
| | 2 H), 7.19 (s, 1 H), 7.21-7.26 (d, 2 H), 7.91-7.93 (d, 2 H), 8.11 (s, 2 H), 8.71 (s, 1 H), 9.56 (s, 1 H). MS (M + 1): 466.3 | | | | | |
| 1BI | —CH₂CH₃ | CH | CH | 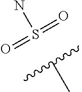 | CH | 5-ethoxy- pyrazin-2-yl |

¹H NMR (400 MHz, DMSO-d6) δ ppm 1.29-1.32 (m, 3 H), 1.46-1.50 (m, 3 H), 4.28-4.33 (m, 2 H), 4.43-4.49 (m, 2 H),
7.08 (s, 1 H), 7.24-7.26 (d, 2 H), 7.34 (s, 2 H), 7.82-7.84 (d, 2 H), 8.11 (d, 1 H), 8.28 (s, 1 H), 8.45 (s, 1 H), 8.84 (s, 1 H),
10.92 (s, 1 H). MS (M + 1): 483.2

### Example 1BK

### Preparation of 4-{4-[(dimethylamino)sulfonyl]phenoxy}-2-methyl-N-(1-methyl-1H-pyrazol-3-yl)-2H-indazole-6-carboxamide (1BK)

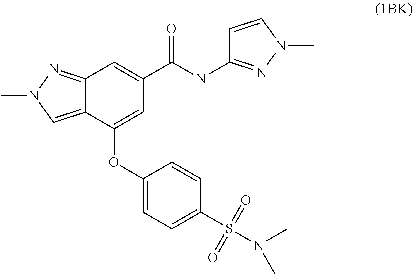

To a solution of 1-methyl-1H-pyrazol-3-ylamine (49.2 mg, 0.51 mmol) in 0.7 mL of dimethoxyethane at room temperature was added trimethylaluminum (2.0M in toluene, 0.51 mL, 1 mmol). After stirring for 1 hour at room temperature under nitrogen, the clear yellow solution was poured into a solution of ethyl 4-{4-[(dimethylamino)sulfonyl]phenoxy}-2-methyl-2H-indazole-6-carboxylate (I-1f-35, 68 mg, 0.17 mmol) in 1 mL of dimethoxyethane under nitrogen and was heated at 90° C. overnight. The crude reaction mixture was taken up in dichloromethane and washed with saturated Rochelle salt solution. After stirring 1 hr, the mixture was separated, the aqueous layer was re-extracted twice with dichloromethane, and the combined organic layers dried over sodium sulfate, filtered, concentrated, and flash chromatographed eluting with a 0 to 8% gradient of methanol in dichloromethane to afford the title compound (1BK: 65.4 mg, 85%).

¹H NMR (400 MHz, CHLOROFORM-d): 6 ppm 2.72 (s, 6H), 3.81 (s, 3H), 4.22 (s, 3H), 6.79 (d, J=1.95 Hz, 1H), 7.16 (d, J=8.79 Hz, 2H), 7.20 (s, 1H), 7.28 (d, J=2.15 Hz, 1H), 7.75 (d, J=8.79 Hz, 2H), 7.79 (s, 1H), 8.02 (s, 1H), 8.51 (br. s., 1H). MS (M+1): 454.9.

The compounds listed in Table 3 below were prepared using procedures analogous to those for the preparation of Compound (1 BK) above using the appropriate starting materials and intermediates described above.

TABLE 3

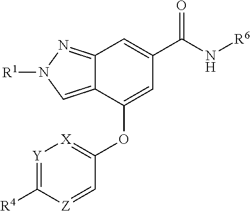

| Example No. | R¹ | X | Y | R⁴ | Z | R⁶ |
|---|---|---|---|---|---|---|
| 1BL | —CH₃ | CH | CH | 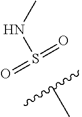 | CH | 1-methyl- pyrazol-3-yl |
| | ¹H NMR (400 MHz, CHLOROFORM-d): δ ppm 2.68 (d, J = 5.47 Hz, 3 H), 3.77 (s, 3 H), 4.22 (s, 3 H), 4.73 (d, J = 5.47 Hz, 1 H), | | | | | |
| | 6.79 (d, J = 2.15 Hz, 1 H), 7.11-7.18 (m, 3 H), 7.28 (d, J = 2.15 Hz, 1 H), 7.77-7.86 (m, 3 H), 8.02 (s, 1 H), 8.73 (s, 1 H). MS | | | | | |
| | (M + 1): 440.9. | | | | | |
| 1BM | —CH₂CH₃ | CH | CH | 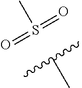 | CH | 1-ethyl-pyrazol- 3-yl |
| | ¹H NMR (400 MHz, METHANOL-d4) δ ppm 1.43 (t, J = 7.33 Hz, 3 H), 1.57 (t, J = 7.33 Hz, 3 H), 3.11 (s, 3 H), 4.10 (q, J = 7.23 Hz, | | | | | |
| | 2 H), 4.49 (q, J = 7.23 Hz, 2 H), 6.58 (d, J = 2.35 Hz, 1 H), 7.19 (d, J = 0.78 Hz, 1 H), 7.23-7.31 (m, 2 H), 7.52 (d, J = 2.35 Hz, 1 | | | | | |
| | H), 7.91-7.99 (m, 2 H), 8.14 (s, 1 H), 8.19 (s, 1 H), NH proton was exchanged. MS (M + 1): 454.4. | | | | | |
| 1BN | —CH₃ | CH | CH | 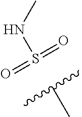 | CH | 5- methoxypyrazin- 2-yl |
| | ¹H NMR (400 MHz, CHLOROFORM-d) ppm 2.70 (br. s., 3 H), 3.98 (s, 3 H), 4.23 (s, 3 H), 4.33 (br. s., 1 H), 7.14-7.16 (m, | | | | | |
| | 1 H), 7.16-7.18 (m, 1 H), 7.20 (d, J = 1.17 Hz, 1 H), 7.81 (s, 1 H), 7.82-7.85 (m, 1 H), 7.84-7.86 (m, 1 H), 7.95 (d, J = 0.98 | | | | | |
| | Hz, 1 H), 8.05-8.09 (m, 1 H), 8.40 (s, 1 H), 9.16 (d, J = 1.37 Hz, 1 H). MS (M + 1): 466.9. | | | | | |
| 1BO | —CH₃ | N | CH | 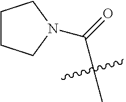 | N | 1-methyl-1H- pyrazol-3-yl |
| | ¹H NMR (400 MHz, CHLOROFORM-d) δ ppm 1.88-2.01 (m, 4 H) 3.69 (t, 2 H) 3.80 (t, 2 H) 3.86 (s, 3 H) 4.22 (s, 3 H) 6.87 | | | | | |
| | (s, 1 H) 7.32 (br. s., 1 H) 7.44 (s, 1 H) 7.73 (s, 1 H) 8.17 (s, 1 H) 8.42 (d, J = 0.98 Hz, 1 H) 8.68 (s, 1 H) 8.95 (br. s., 1 H). | | | | | |
| | MS (M + 1): 447.4 | | | | | |
| 1BP | —CH₂CH₃ | N | CH | 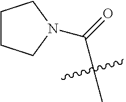 | N | 5-methyl- pyrazin-2-yl |
| | ¹H NMR (400 MHz, CHLOROFORM-d) δ ppm 1.67 (t, J = 7.4 Hz, 3 H), 1.93-2.02 (m, 4 H), 2.58 (s, 3 H), 3.68-3.74 (m, 2 | | | | | |
| | H), 3.80-3.87 (m, 2 H), 4.51 (q, J = 7.3 Hz, 2 H), 7.47 (d, J = 1.2 Hz, 1 H), 7.81 (s, 1 H), 8.16-8.19 (m, 1 H), 8.20-8.23 | | | | | |
| | (m, 1 H), 8.47 (d, J = 1.4 Hz, 1 H), 8.49-8.53 (m, 1 H), 8.72 (d, J = 1.4 Hz, 1 H), 9.59 (d, J = 1.6 Hz, 1 H). MS (M + 1): 473.2 | | | | | |
| 1BQ | —CH₂CH₃ | N | CH | 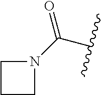 | N | 1-methyl-1H- pyrazol-3-yl |
| | ¹H NMR (400 MHz, CHLOROFORM-d) δ ppm 1.59-1.65 (m, 3 H), 2.37 (quin, J = 7.71 Hz, 2 H), 3.80 (s, 3 H), 4.25 (t, | | | | | |
| | J = 7.80 Hz, 2 H), 4.47 (q, J = 7.28 Hz, 2 H), 4.68 (t, J = 7.61 Hz, 2 H), 6.81 (d, J = 2.15 Hz, 1 H), 7.28 (d, J = 2.34 Hz, 1 H), 7.41 | | | | | |
| | (s, 1 H), 7.75 (s, 1 H), 8.12 (s, 1 H), 8.39 (s, 1 H), 8.58 (s, 1 H), 8.84 (s, 1 H). MS (M + 1): 447.4 | | | | | |
| 1BR | —CH₂CH₃ | N | CH | 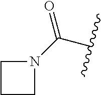 | N | 5-methyl- pyrazin-2-yl |
| | ¹H NMR (400 MHz, CHLOROFORM-d) δ ppm 1.66 (t, J = 7.3 Hz, 3 H), 2.39-2.45 (m, 2 H), 2.58 (s, 3 H), 4.24-4.32 (m, 2 | | | | | |
| | H), 4.51 (q, J = 7.2 Hz, 2 H), 4.67-4.74 (m, 2 H), 7.47 (d, J = 1.2 Hz, 1 H), 7.90-7.92 (m, 1 H), 8.17 (s, 1 H), 8.20-8.24 | | | | | |
| | (m, 1 H), 8.44 (d, J = 1.4 Hz, 1 H), 8.48-8.54 (m, 1 H), 8.86 (d, J = 1.4 Hz, 1 H), 9.59 (d, J = 1.6 Hz, 1 H). MS (M + 1): 459.2 | | | | | |
| 1BS | —CH₃ | N | CH | 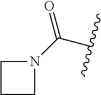 | N | 1-methyl-1H- pyrazol-3-yl |
| | ¹H NMR (400 MHz, CHLOROFORM-d) δ ppm 2.38 (d, J = 7.41 Hz, 2 H), 3.80 (s, 3 H), 4.21 (s, 3 H), 4.22-4.31 (m, 2 | | | | | |
| | H), 4.61-4.75 (m, 2 H), 6.81 (s, 1 H), 7.28 (d, J = 1.95 Hz, 1 H), 7.41 (s, 1 H), 7.70 (s, 1 H), 8.11 (s, 1 H), 8.39 (s, 1 H), | | | | | |
| | 8.60 (br. s., 1 H), 8.83 (d, J = 0.98 Hz, 1 H). MS (M + 1): 433.4 | | | | | |
| 1BT | —CH(CH₃)₂ | N | CH | 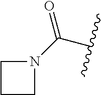 | N | 1-methyl-1H- pyrazol-3-yl |
| | ¹H NMR (400 MHz, CHLOROFORM-d) δ ppm 1.64 (d, J = 6.63 Hz, 6 H), 2.31-2.44 (m, 2 H), 3.84 (s, 3 H), 4.24 (t, 2 | | | | | |
| | H), 4.68 (t, 2 H), 4.73-4.84 (m, 1 H), 6.85 (s, 1 H), 7.31 (s, 1 H), 7.41 (s, 1 H), 7.79 (s, 1 H), 8.17 (s, 1 H), 8.40 (s, 1 H), | | | | | |
| | 8.84 (s, 1 H). MS (M + 1): 461.4 | | | | | |
| 1BU | —CH₂CH₃ | N | CH | —C(O)N(CH₃)₂ | N | 1-methyl-1H- |
| | | | | | | pyrazol-3-yl |
| | ¹H NMR (400 MHz, CHLOROFORM-d) δ ppm 1.62 (t, 3 H), 3.15 (d, J = 10.34 Hz, 6 H), 3.77 (s, 3 H), 4.47 (q, J = 7.28 Hz, | | | | | |
| | 2 H), 6.81 (d, J = 2.15 Hz, 1 H), 7.27 (d, J = 2.15 Hz, 1 H), 7.41 (d, J = 1.17 Hz, 1 H), 7.77 (s, 1 H), 8.13 (s, 1 H), 8.42 (d, | | | | | |
| | J = 1.37 Hz, 1 H), 8.49 (d, J = 1.17 Hz, 1 H), 8.77 (s, 1 H). MS (M + 1): 435.3 | | | | | |
| 1BV | —CH(CH₃)₂ | N | CH | —C(O)N(CH₃)₂ | N | 1-methyl-1H- |
| | | | | | | pyrazol-3-yl |
| | ¹H NMR (400 MHz, CHLOROFORM-d) δ ppm 1.63 (d, J = 6.64 Hz, 6 H), 3.15 (d, J = 9.97 Hz, 6 H), 3.78 (s, 3 H), 4.71- | | | | | |
| | 4.84 (m, 1 H), 6.80 (br. s., 1 H), 7.27 (s, 1 H), 7.39 (s, 1 H), 7.81 (s, 1 H), 8.12 (s, 1 H), 8.42 (s, 1 H), 8.49 (s, 1 H), 8.58 | | | | | |
| | (s, 1 H). MS (M + 1): 449.4 | | | | | |
| 1BW | —CH₃ | N | CH | 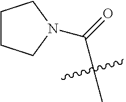 | N | 2-methyl-2H- 1,2,3-triazol-4-yl |
| | ¹H NMR (400 MHz, CHLOROFORM-d) δ ppm 1.95 (br. s., 4 H), 3.69 (br. s., 2 H), 3.81 (br. s., 2 H), 4.10 (s, 3 H), 4.23 (s, | | | | | |
| | 3 H), 7.48 (s, 1 H), 7.74 (s, 1 H), 8.16 (s, 1 H), 8.25 (s, 1 H), 8.42 (s, 1 H), 8.68 (s, 1 H), 9.43 (s, 1 H). MS (M + 1): 448.4 | | | | | |
| IBX | —CH₂CH₃ | N | CH | 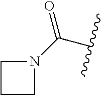 | N | 2-methyl-2H- 1,2,3-triazol-4-yl |
| | ¹H NMR (400 MHz, CHLOROFORM-d) δ ppm 1.62 (t, 3 H), 2.36 (quin, J = 7.76 Hz, 2 H), 4.06 (s, 3 H), 4.24 (t, J = 7.80 Hz, | | | | | |
| | 2 H), 4.47 (q, J = 7.28 Hz, 2 H), 4.66 (t, J = 7.71 Hz, 2 H), 7.50 (d, J = 1.17 Hz, 1 H), 7.75 (s, 1 H), 8.16 (s, 1 H), 8.31 (s, 1 H), | | | | | |
| | 8.38 (d, J = 1.37 Hz, 1 H), 8.83 (d, J = 1.37 Hz, 1 H), 9.84 (s, 1 H). MS (M + 1): 448.3 | | | | | |
| 1BY | —CH(CH₃)₂ | N | CH | 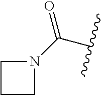 | N | 2-methyl-2H- 1,2,3-triazol-4-yl |
| | ¹H NMR (400 MHz, CHLOROFORM-d) δ ppm 1.65 (d, J = 6.63 Hz, 6 H), 2.31-2.45 (m, 2 H), 4.12 (s, 3 H), 4.26 (t, 2 | | | | | |
| | H), 4.68 (t, 2 H), 4.75-4.85 (m, 1 H), 7.44 (s, 1 H), 7.80 (s, 1 H), 8.18 (s, 1 H), 8.26 (s, 1 H), 8.41 (s, 1 H), 8.85 (s, 1 H), | | | | | |
| | 9.24 (s, 1 H). MS (M + 1): 462.4 | | | | | |
| 1BZ | —CH₂CH₃ | N | CH | —C(O)N(CH₃)₂ | N | 2-methyl-2H- |
| | | | | | | 1,2,3-triazol-4-yl |

¹H NMR (400 MHz, CHLOROFORM-d) δ ppm 1.62 (t, 3 H), 3.14 (d, J = 8.58 Hz, 6 H), 4.06 (s, 3 H), 4.47 (q, J = 7.41 Hz, 2
H), 7.51 (d, J = 1.17 Hz, 1 H), 7.78 (s, 1 H), 8.17 (s, 1 H), 8.33 (d, J = 0.98 Hz, 1 H), 8.42 (d, J = 1.37 Hz, 1 H), 8.49 (d, J = 1.37
Hz, 1 H), 10.01 (s, 1 H). MS (M + 1): 436.1

### Example 1CA

### Preparation of 2-ethyl-4-[4-(methylsulfonyl)phenoxy]-N-(2-methyl-2H-1,2,3-triazol-4-yl)-2H-indazole-6-carboxamide (1CA)

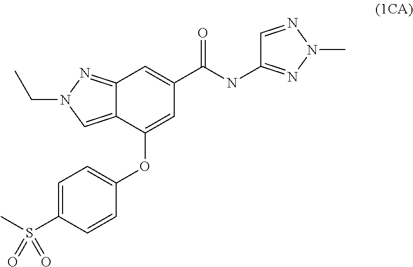

To a suspension of 2-methyl-2H-1,2,3-triazol-4-amine (157 mg, 1.60 mmol) in dry dichloroethane (2.0 mL) was added trimethylaluminum (0.8 mL, 1.6 mmol) slowly at 0° C. under nitrogen. After addition, the mixture was stirred for 1 h at room temperature and then added methyl 2-ethyl-4-(4-(methylsulfonyl)phenoxy)-2H-indazole-6-carboxylate (I-1f-7, 120 mg, 0.32 mmol). The mixture was heated to 60-70° C. overnight. The reaction mixture was cooled to room temperature and poured into potassium sodium tartrate (aqueous, 10 mL). Then the mixture was extracted with ethyl acetate (3×10 mL). The combined organic layers were dried and concentrated under reduced pressure and purified by preparative HPLC to afford the title compound (1CA: 83.1 mg, 58.9%) as a white solid.

¹H NMR (400 MHz, DMSO-d6): δ 1.46-1.50 (m, 3H), 3.20 (s, 3H), 4.02 (s, 3H), 4.43-4.49 (m, 2H), 7.25-7.29 (m, 3H), 7.91-7.93 (d, 2H), 8.24 (s, 1H), 8.33 (s, 1H), 8.42 (s, 1H), 11.36 (s, 1H). MS (M+23): 463.3.

The compounds listed in Table 4 below were prepared using procedures analogous to those for the preparation of Compound (1CA) above using the appropriate starting materials and intermediates described above.

TABLE 4

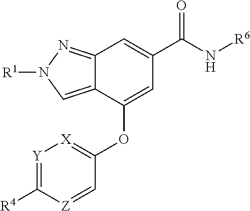

| Example No. | R¹ | X | Y | R⁴ | Z | R⁶ |
|---|---|---|---|---|---|---|
| 1CB | —CH₂CH₃ | CH | CH | 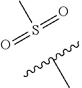 | CH | 5-methyl-pyrazin- 2-yl |
| | ¹H NMR (400 MHz, DMSO d6): δ 1.47-1.51 (m, 3 H), 2.45 (s, 3 H), 3.20 (s, 3 H), 4.44-4.50 (m, 2 H), 7.22 (s, 1 H), 7.28- | | | | | |
| | 7.30 (d, 2 H), 7.92-7.94 (d, 2 H), 8.34 (d, 2 H), 8.44 (s, 1 H), 9.23 (s, 1 H), 11.07 (s, 1 H). MS (M + 1): 452.0. | | | | | |
| 1CC | —CH₂CH₃ | CH | CH | 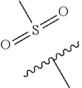 | CH | 1-methyl-pyrazol- 3-yl |
| | ¹H NMR (400 MHz, DMSO d6): 6 1.45-1.48 (m, 3 H), 3.17 (s, 3 H), 3.74 (s, 3 H), 4.41-4.47 (m, 2 H), 6.53 (d, 2 H), 7.18 | | | | | |
| | (s, 1 H), 7.26-7.28 (d, 2 H), 7.56 (d, 2 H), 7.90-7.92 (d, 2 H), 8.24 (s, 1 H), 8.40 (s, 1 H), 10.87 (s, 1 H). MS (M + 1): 440.2. | | | | | |
| 1CD | —CH₂CH₃ | CH | CH | 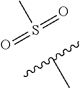 | CH | 5-carboxamido- pyridin-2-yl |
| | ¹H NMR (400 MHz, DMSO d6): δ 1.47-1.50 (m, 3 H), 3.20 (s, 3 H), 4.40-4.93 (m, 2 H), 7.22 (s, 1 H), 7.28-7.30 (d, 2 H), | | | | | |
| | 7.46 (s, 1 H), 7.92-7.94 (d, 2 H), 8.05 (s, 1 H), 8.20-8.26 (m, 2 H), 8.34 (s, 1 H), 8.43 (s, 1 H), 8.84 (d, 1 H), 11.50 (s, 1 H). MS | | | | | |
| | (M + 1): 480.2. | | | | | |
| 1CE | —CH₂CH₃ | CH | CH | 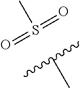 | CH | 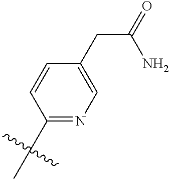 |
| | ¹H NMR (400 MHz, DMSO d6): δ 1.45-1.49 (m, 3 H), 3.18 (s, 3 H), 3.36 (s, 2 H), 4.42-4.48 (m, 2 H), 6.92 (s, 1 H), 7.21 | | | | | |
| | (s, 1 H), 7.26-7.28 (d, 2 H), 7.50 (s, 1 H), 7.65-7.68 (m, 1 H), 7.90-7.93 (d, 2 H), 8.04-8.07 (d, 1 H), 8.21 (s, 1 H), 8.31 (s, 1 H), | | | | | |
| | 8.41 (d, 1 H), 10.84 (s, 1 H). MS (M + 1): 494.2. | | | | | |
| 1CG | —CH₂CH₃ | CH | CH | 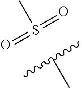 | CH | 5-fluoro-pyridin- 2-yl |
| | ¹H NMR (400 MHz, DMSO d6): δ 1.46 (t, J = 7.2 Hz, 3 H), 3.16 (s, 3 H), 4.43 (q, J = 7.2 Hz, 2 H), 4.62 (s, 2 H), 6.55 (d, J = | | | | | |
| | 2.4 Hz, 1 H), 7.16 (s, 1 H), 7.20 (s, 1 H), 7.26 (d, J = 8.8 Hz, 2 H), 7.40 (s, 1 H), 7.57 (d, J = 2.4 Hz, 1 H), 7.90 (d, J = 8.8 | | | | | |
| | Hz, 2 H), 8.23 (s, 1 H), 8.38 (s, 1 H), 10.92 (s, 1 H). MS (M + 1): 483.2. | | | | | |
| 1CI | —CH₂CH₃ | CH | CH | 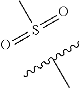 | CH | 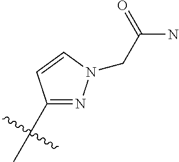 |
| | ¹H NMR (400 MHz, DMSO-d6) δ ppm 1.49-1.52 (t, 3 H), 3.13 (s, 3 H), 4.39-4.40 (q, 2 H), 7.14 (s, 1 H), 7.20-7.23 (d, 2 H), | | | | | |
| | 7.70-7.71 (m, 1 H), 7.85-7.87 (d, 2 H), 8.08-8.11 (m, 1 H), 8.25 (s, 1 H), 8.31-8.32 (d, 1 H), 8.36 (s, 1 H), 10.93 (s, 1 H. MS | | | | | |
| | (M + 1): 455.2 | | | | | |
| 1CJ | —CH₃ | CH | CH | 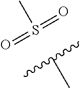 | CH | 5-fluoropyridin-2- yl |

¹H NMR (400 MHz, DMSO-d6) δ ppm 3.21 (s, 3 H), 4.04 (s, 3 H), 4.18 (s, 3 H), 7.94 (d, 2 H), 8.26 (s, 1 H), 8.35 (s, 2 H),
11.42 (s, 1 H). MS (M + 1): 427.1

### Example 1CK

### Preparation of methyl 6-[({2-ethyl-4-[4-(methylsulfonyl)phenoxy]-2H-indazol-6-yl}carbonyl)amino]nicotinate (1CK)

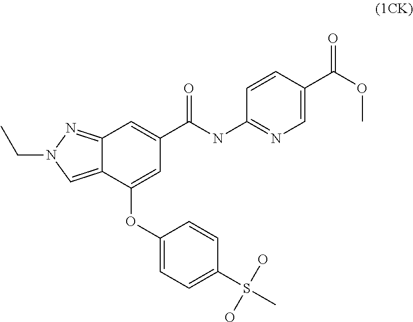

To a solution of 2-ethyl-4-[4-(methylsulfonyl)phenoxy]-2H-indazole-6-carboxylic acid (I-1g-3: 30 mg, 0.083 mmol), methyl-6-aminonicotinate (18.9 mg, 0.124 mmol), 2-(1H-benzotriazol-1-yl)1,1,3,3-tetramethyluronium tetrafluoroborate (82 mg, 0.248 mmol), and triethylamine (50.4 mg, 0.500 mmol) in N,N-dimethylformamide (0.28 mL), was heated to 50° C. for 2 hours. The reaction mixture was diluted with ethyl acetate (1 ml), washed twice with water (0.5 ml), dried over sodium sulfate, filtered and concentrated. The crude material was separated by flash chromatography eluting with a gradient of 10-100% ethyl acetate in heptane followed by a second to give flash chromatography eluting with a gradient of 2-20% methanol in dichloromethane to afford the title compound methyl 6-[({2-ethyl-4-[4-(methylsulfonyl)phenoxy]-2H-indazol-6-yl}carbonyl)amino]nicotinate (1CK: 6 mg, 10%) as a white solid.

¹H NMR (400 MHz, CHLOROFORM-d) ppm 1.64 (t, J=7.33 Hz, 3 H), 3.08 (s, 3H), 3.93 (s, 3H), 4.49 (q, J=7.30 Hz, 2H), 7.16-7.23 (m, 3H), 7.85 (s, 1H) 7.93 (d, 2H), 8.13 (s, 1H), 8.31-8.37 (m, 1H), 8.42 (d, 1H), 8.79 (s, 1H), 8.93 (d, J=1.56 Hz, 1H). MS (M+1): 495.1.

### Example 1CL

### Preparation of 2-ethyl-4-[4-(methylsulfonyl)phenoxy]-N-1,2,4-thiadiazol-5-yl-2H-indazole-6-carboxamide (1CL)

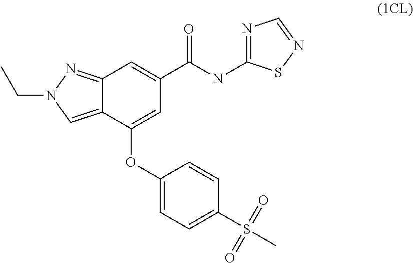

To a solution of 2-ethyl-4-[4-(methylsulfonyl)phenoxy]-2H-indazole-6-carboxylic acid (I-1g-3: 8 mg, 0.02 mmol), 2-amino thiazole (2.2 mg, 0.022 mmol), 2-(1H-benzotriazol-1-yl)1,1,3,3-tetramethyluronium tetrafluoroborate (10.9 mg, 0.033 mmol), and triethylamine (6.7 mg, 0.009 mmol) in N,N-dimethylformamide (0.22 mL), was stirred at room temperature for 60 hours. The reaction mixture was concentrated, then purified by preparative thin layer chromatography eluting with ethyl acetate to afford the title compound 2-ethyl-4-[4-(methylsulfonyl)phenoxy]-N-1,2,4-thiadiazol-5-yl-2H-indazole-6-carboxamide (1CL: 2 mg, 20%).

¹H NMR (400 MHz, METHANOL-d4) ppm 1.60 (t, J=7.33 Hz, 3H), 3.12 (s, 3H), 4.46-4.58 (m, 2H), 7.26-7.32 (m, 2H), 7.78 (s, 2H), 7.82 (s, 1H), 7.94-7.97 (m, 1H), 7.97-8.00 (m, 1H), 8.22 (s, 1H), 8.34-8.39 (m, 1H). MS (M+1): 444.1.

### Example 1CM

### Preparation of 4-(5-acetamidopyrazin-2-yloxy)-2-ethyl-N-(5-methylpyridin-2-yl)-2H-indazole-6-carboxamide (1CM)

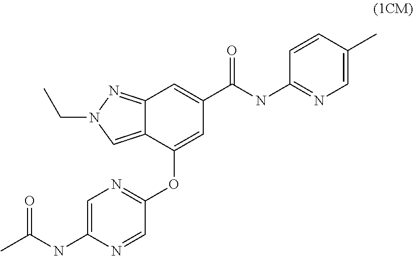

To a solution of 5-methylpyridin-2-amine (24.9 mg, 0.230 mmol) in 1,2-dimethoxyethane (1.5 mL) was added dimethylaluminum chloride (1.0M in hexanes) (0.461 mL). The reaction was stirred for 15 minutes at room temperature. Ethyl 4-(5-acetamidopyrazin-2-yloxy)-2-ethyl-2H-indazole-6-carboxylate (I-1h-1: 57 mg, 0.15 mmol) in 1,2-dimethoxyethane (1.5 mL) was then added and the reaction was heated to 70° C. in a sealed tube for 2 hours. The reaction was cooled to room temperature and diluted with ethyl acetate and saturated Rochelle's salt. The mixture was left stirring overnight. The mixture was extracted with ethyl acetate and the combined organics were washed with brine, dried over sodium sulfate, filtered, and concentrated. The crude residue was dissolved in dichloromethane (1.44 mL). Pyridine (0.118 mL, 1.44 mmol) and acetyl chloride (5.0 uL, 0.072 mmol) were added and the reaction was stirred at room temperature for 5 minutes. The reaction was concentrated and purified by column chromatography to afford the title compound 4-(5-acetamidopyrazin-2-yloxy)-2-ethyl-N-(5-methylpyridin-2-yl)-2H-indazole-6-carboxamide (1CM: 14.3 mg, 46%) as a yellow solid.

¹H NMR (400 MHz, CHLOROFORM-d) δ ppm 1.64 (t, 3H), 2.23-2.25 (m, 3H), 2.34 (s, 3H), 4.48 (q, J=7.30 Hz, 2H), 7.42 (s, 1H), 7.62-7.73 (m, 1H), 7.86 (s, 1H), 8.03 (s, 1H), 8.05-8.10 (m, 1H), 8.17 (s, 1H), 8.35 (s, 1H), 8.44 (d, J=8.40 Hz, 1H), 9.11 (s, 1H). MS (M+1): 432.5.

### Example 1CN

### Preparation of 2-methyl-4-(5-(N-methylacetamido)pyrazin-2-yloxy)-N-(5-methylpyridin-2-yl)-2H-indazole-6-carboxamide (1CN)

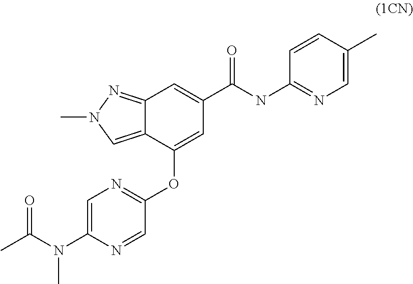

The title compound was prepared by a method analogous to that described for Example 1CM, using methyl 2-methyl-4-(5-(N-methylacetamido)pyrazin-2-yloxy)-2H-indazole-6-carboxylate (I-1f-15).

MS (M+1): 432.17 RT 2.04 min. Column: Waters XBridge C18 4.6×50 mm, 5 μm. Modifier: Ammonium hydroxide 0.03%. Gradient: 90% H2O/10% MeCN linear to 5% H2O/95% MeCN over 4 min, held to 5.0 min. Flow rate: 2.0 mL/min.

### Example 1CO

### Preparation of 4-(5-carbamoylpyrazin-2-yloxy)-2-ethyl-N-(5-methylpyridin-2-yl)-2H-indazole-6-carboxamide (1CO)

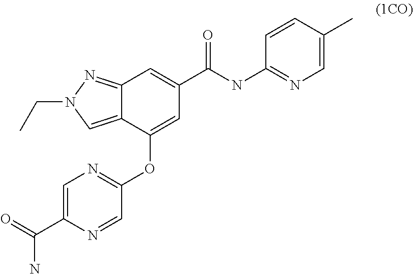

To a solution of 4-(5-(bis(2,4-dimethoxybenzyl)carbamoyl)pyrazin-2-yloxy)-2-ethyl-N-(5-methylpyridin-2-yl)-2H-indazole-6-carboxamide (I-1g-2: 75 mg, 0.10 mmol) in dichloromethane (1.04 mL) was added triethylsilane (50 μL, 0.312 mmol) and trifluoroacetic acid (0.160 mL, 2.08 mmol). The reaction was stirred at room temperature for 2 days. LCMS showed some mono-protected intermediate remaining. Another 20 equivalents of trifluoroacetic acid (0.160 mL, 2.08 mmol) were added. After stirring for 2 more days, the reaction was concentrated. The crude was taken up in dichloromethane, washed with saturated sodium bicarbonate, water, and brine. The organics were dried over sodium sulfate, filtered, and concentrated. Column chromatography (10-100% ethyl acetate in heptane, then 3% methanol in ethyl acetate) afforded the title compound 4-(5-carbamoyl pyrazin-2-yloxy)-2-ethyl-N-(5-methylpyridin-2-yl)-2H-indazole-6-carboxamide (100: 14.8 mg, 34%) as a white solid.

¹HNMR (400 MHz, DMSO-d6) δ ppm 1.47 (t, J=7.22 Hz, 3H) 2.26 (s, 3H) 4.43 (q, J=7.22 Hz, 2H) 7.46 (d, 1H) 7.63 (d, 1H) 7.72 (d, 1H) 8.04 (d, 1H) 8.12 (d, 1H) 8.20 (d, 1H) 8.35 (d, 2H) 8.66 (d, 2H) 10.78 (s, 1H). MS (M−1): 416.1.

### Example 1CP

### Preparation of 2-ethyl-N-(5-methylpyridin-2-yl)-4-(5-(morpholine-4-carbonyl)pyrazin-2-yloxy)-2H-indazole-6-carboxamide (1CP)

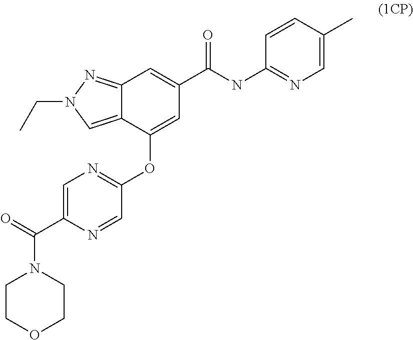

To a solution of 2-ethyl-4-hydroxy-N-(5-methylpyridin-2-yl)-2H-indazole-6-carboxamide (I-2f-1: 27.0 mg, 0.091 mmol) in dimethylformamide (0.91 mL) was added (5-chloropyrazin-2-yl)(morpholino)methanone (WO2008099000) (41.4 mg, 0.182 mmol) and potassium carbonate (25.2 mg, 0.182 mmol). The reaction was stirred at 80° C. for 2 hours. The reaction was diluted with ethyl acetate (5 mL) and washed with water (3×1 mL). The organics were dried over magnesium sulfate, filtered, and concentrated. Column chromatography (10-100% ethyl acetate in heptane) afforded the title compound 2-ethyl-N-(5-methylpyridin-2-yl)-4-(5-(morpholine-4-carbonyl)pyrazin-2-yloxy)-2H-indazole-6-carboxamide (1CP: 23 mg, 52%).

¹H NMR (400 MHz, CHLOROFORM-d) δ ppm 1.64 (t, J=7.33 Hz, 3H), 2.31 (s, 3H), 3.77 (d, 8H), 4.48 (q, 2H), 7.42 (s, 1H), 7.56 (d, 1H), 7.80 (s, 1H), 8.11 (s, 1H), 8.18 (s, 1H), 8.25 (d, 1H), 8.42 (s, 1H), 8.54 (s, 2H). MS (M+1): 488.5.

### Example 1CQ

### Preparation of 4-[4-(aminosulfonyl)phenoxy]-2-ethyl-N-(1-methyl-1H-pyrazol-3-yl)-2H-indazole-6-carboxamide (1CQ)

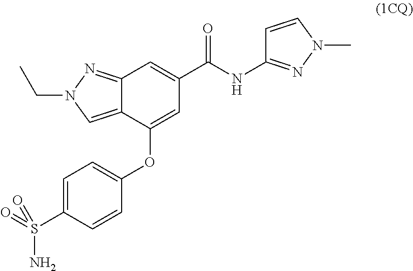

To a solution of 1-methyl-1H-pyrazol-3-amine (86.4 mg, 0.890 mmol) in dry dichloroethane (0.5 mL) was added trimethylaluminum (0.890 mL, 1.78 mmol) at 0° C. The reaction was allowed to warm to room temperature and stir. After 1 hour, the mixture was added to a solution of methyl 4-(4-(N,N-bis(2,4-dimethoxybenzyl)sulfamoyl)phenoxy)-2-ethyl-2H-indazole-6-carboxylate (I-1f-32: 120.0 mg, 0.178 mmol) in dry dichloroethane (0.5 mL) and stirred at 60° C. for 8 hours. The reaction mixture was then poured into an aqueous solution of potassium sodium tartrate. Then the mixture was extracted with ethyl acetate (3×10 mL). The combined organic layers were dried and concentrated under reduced pressure to give a crude residue (440 mg).

To a solution of the crude residue (440 mg, 0.584 mmol) in dry dichloromethane (4.0 mL) was added trifluoroacetic acid (8.0 mL). The reaction was stirred at 30° C. for 4 hours. The mixture was diluted with water (15 mL) and then aqueous potassium carbonate was added until no further gas evolution was observed. The mixture was extracted with ethyl acetate (3×15 mL). The combined organic layers were dried and concentrated under reduced pressure. Purification by HPLC afforded the title compound, 4-[4-(aminosulfonyl)phenoxy]-2-ethyl-N-(1-methyl-1H-pyrazol-3-yl)-2H-indazole-6-carboxamide (1CQ: 29.4 mg, 11.4%) as a white solid.

¹HNMR (400 MHz, DMSO-d6) δ ppm 1.44-1.48 (m, 3H), 3.73 (s, 3H), 4.41-4.46 (m, 2H), 6.51 (s, 1H), 7.04 (s, 1H), 7.22-7.24 (d, 2H), 7.32 (s, 2H), 7.55 (d, 1H), 7.80-7.83 (d, 2H), 8.19 (s, 1H), 8.41 (s, 1H), 10.86 (s, 1H). MS (M+1): 441.3

### Example 1CR

### Preparation of 4-[4-(aminosulfonyl)phenoxy]-2-ethyl-N-(5-methylpyrazin-2-yl)-2H-indazole-6-carboxamide (1 CR)

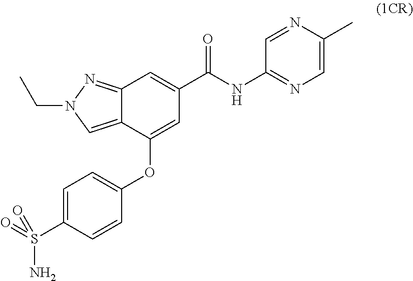

To a solution of 5-methylpyrazin-2-amine (96.9 mg, 0.89 mmol) in dry dichloroethane (0.5 mL) was added dimethylaluminum chloride (1.48 mL, 1.48 mmol) at 0° C. The solution was allowed to stir at room temperature for 1 hour. The solution was then added to a mixture of methyl 4-(4-(N,N-bis(2,4-dimethoxybenzyl) sulfamoyl)phenoxy)-2-ethyl-2H-indazole-6-carboxylate (I-1f-32: 100.0 mg, 0.128 mmol) in dry dichloroethane (0.5 mL) and stirred at 60° C. for 30 min. The reaction mixture was poured into aqueous potassium sodium tartrate. Then the mixture was extracted with ethyl acetate (3×10 mL). The combined organics were dried and concentrated under reduced pressure to give crude residue (340 mg). The crude residue was then treated with trifluoroacetic acid as previously described in Example 1CQ, to afford the title compound 4-[4-(aminosulfonyl)phenoxy]-2-ethyl-N-(5-methylpyrazin-2-yl)-2H-indazole-6-carboxamide (1CR: 30.5 mg, 14.9%) as a white solid.

¹HNMR (400 MHz, DMSO-d6) δ ppm 1.46-1.50 (m, 3H), 2.43 (s, 3H), 4.43-4.49 (m, 2H), 7.08 (d, 1H), 7.26-7.27 (d, 2H), 7.34 (s, 2H), 7.82-7.84 (d, 2H), 8.30-8.32 (d, 2H), 8.46 (s, 1H), 9.21 (d, 1H), 11.06 (s, 1H). MS (M+1): 523.2

### Example 1CS

### Preparation of 4-[4-(aminosulfonyl)phenoxy]-2-ethyl-N-(5-methoxypyrazin-2-yl)-2H-indazole-6-carboxamide (1CS)

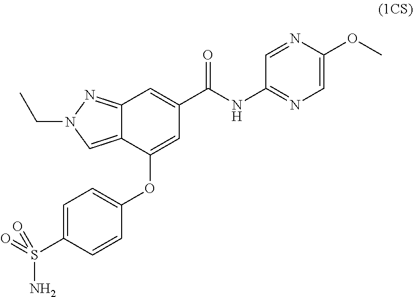

The title compound was prepared by a method analogous to that described for Example 1CR, using 2-amino-5-methoxypyrazine.

¹HNMR (400 MHz, DMSO-d6) δ ppm 1.47-1.51 (m, 3H), 3.89 (s, 3H), 4.44-4.50 (m, 2H), 7.09 (s, 1H), 7.25-7.27 (d, 2H), 7.34 (s, 2H), 7.83-7.85 (d, 2H), 8.15 (d, 1H), 8.29 (s, 1H), 8.46 (s, 1H), 8.87 (d, 1H), 10.95 (s, 1H). MS (M+1): 469.3

### Example 1CT

### Preparation of 4-[4-(aminosulfonyl)phenoxy]-2-ethyl-N-(5-fluoropyridin-2-yl)-2H-indazole-6-carboxamide (1CT)

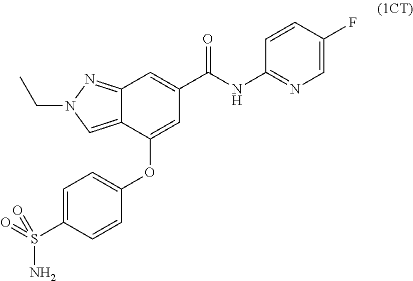

The title compound was prepared by a method analogous to that described for Example 1CR, using 5-fluoropyridin-2-amine.

¹HNMR (400 MHz, DMSO-d6) δ ppm 1.47-1.50 (m, 3H), 4.44-4.49 (m, 2H), 7.08 (s, 1H), 7.24-7.26 (d, 2H), 7.34 (s, 2H), 7.74-7.79 (m, 1H), 7.82-7.84 (d, 2H), 8.13-8.16 (m, 1H), 8.27 (s, 1H), 8.37 (d, 1H), 8.44 (s, 1H), 10.98 (s, 1H). MS (M+1): 456.2

### Example 1CU

### Preparation of 4-[4-(aminosulfonyl)phenoxy]-2-ethyl-N-(5-ethylpyrazin-2-yl)-2H-indazole-6-carboxamide (1CU)

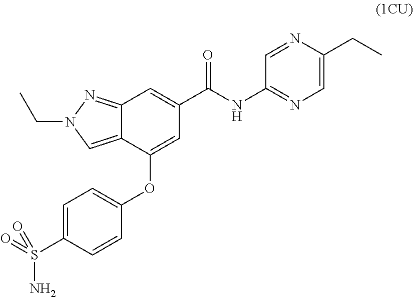

The title compound was prepared by a method analogous to that described for Example 1CR, using 5-ethylpyrazin-2-amine.

¹HNMR (400 MHz, DMSO-d6) δ ppm 1.20-1.23 (m, 3H), 1.47-1.51 (m, 3H), 2.72-2.78 (m, 2H), 4.44-4.50 (m, 2H), 7.09 (s, 1H), 7.25-7.27 (d, 2H), 7.34 (s, 2H), 7.83-7.85 (d, 2H), 8.11 (s, 1H), 8.35 (s, 1H), 8.46 (s, 1H), 9.24 (d, 1H), 11.08 (s, 1H). MS (M+1): 467.1

### Example 1CV

### Preparation of 2-ethyl-N-(5-methylpyridin-2-yl)-4-[4-(S-methylsulfonimidoyl) phenoxy]-2H-indazole-6-carboxamide (1CV)

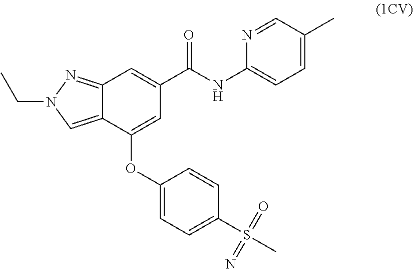

5-methylpyridine-2-amine (22.3 mg, 0.206 mmol) was dissolved in 1 mL dichloroethane and dimethylaluminum chloride solution (0.227 mL, 0.227 mmol) was added at room temperature. After 30 minutes at room temperature, a solution of ethyl 4-{4-[N-(tert-butoxycarbonyl)-S-methylsulfonimidoyl]phenoxy}-2-ethyl-2H-indazole-6-carboxylate (I-1f-42) was added to the aluminum amide solution with stirring at room temperature. The mixture was heated to reflux with stirring under dry nitrogen. After 1 hour, an additional 2 equivalents of 5-methylpyridine-2-amine and 2.5 equivalents of dimethylaluminum chloride was dissolved in 0.5 mL dichloroethane and added to the refluxing mixture. After addition, the mixture was heated for an additional 90 minutes. Added 20 mL Rochelle's salt solution to the cooled reaction mixture. Ethyl acetate was added and mixture was vigorously stirred for 15 minutes. The organic layer was washed with brine, dried over magnesium sulfate, filtered, concentrated and purified by preparative thin layer chromatography eluting first with a 10% solution of methanol in dichloromethane followed by ethyl acetate to afford the title compound (1CV: 40 mg, 86%) as a white solid.

¹H NMR (400 MHz, CHLOROFORM-d) ppm 1.65 (t, 3H), 2.30 (s, 3H), 3.14 (s, 3H), 4.50 (q, 2H), 7.16-7.23 (m, 3H), 7.56 (dd, 1H), 7.89 (bs, 1H), 7.97-8.03 (m, 2H), 8.09-8.13 (m, 2H), 8.72 (bs, 1H), 8.25 (d, 1H). MS (M+1): 450.4.

### Example 1CW

### Preparation of 2-ethyl-N-(5-methylpyridin-2-yl)-4-[4-(S-methylsulfonimidoyl) phenoxy]-2H-indazole-6-carboxamide (1CW(+)enantiomer)

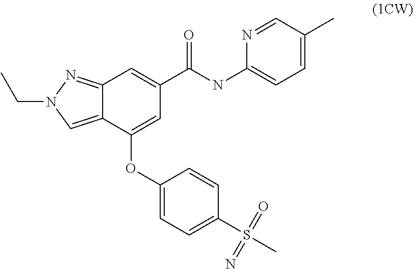

A sample of 1CV (26.7 mg) was subjected to chiral supercritical fluid chromatography eluting with 35% methanol in carbon dioxide containing 0.2% isopropyl amine to separate enantiomers. The second peak to elute was concentrated to afford 1 CW(+)enantiomer (10.6 mg) as determined by qualitative optical rotation.

¹H NMR (400 MHz, CHLOROFORM-d) ppm 1.67 (t, 3H), 2.32 (s, 3H), 2.70 (bs, 1H), 3.15 (s, 3H), 4.51 (q, 2H), 7.17-7.24 (m, 3H), 7.58 (dd, 1H), 7.90 (bs, 1H), 7.99-8.04 (m, 2H), 8.11-8.15 (m, 2H), 8.25 (d, 1H), 8.60 (bs, 1H).

### Example 1CX

### Preparation of 2-ethyl-N-(5-methylpyridin-2-yl)-4-[4-(S-methylsulfonimidoyl phenoxy]-2H-indazole-6-carboxamide (1CX (−)enantiomer)

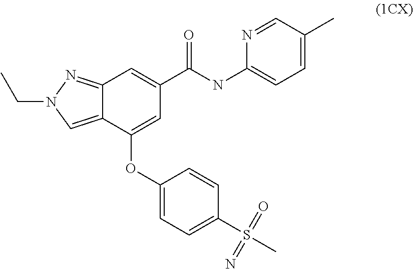

A sample of 1CV (26.7 mg) was subjected to chiral supercritical fluid chromatography eluting with 35% methanol in carbon dioxide containing 0.2% isopropyl amine to separate enantiomers. The first peak to elute was concentrated to afford 1CW(−)enantiomer (12 mg) as determined by qualitative optical rotation.

¹H NMR (400 MHz, CHLOROFORM-d) ppm 1.67 (t, 3H), 2.32 (s, 3H), 2.70 (bs, 1H), 3.15 (s, 3H), 4.51 (q, 2H), 7.17-7.24 (m, 3H), 7.58 (dd, 1H), 7.90 (bs, 1H), 7.99-8.04 (m, 2H), 8.11-8.15 (m, 2H), 8.25 (d, 1H), 8.61 (bs, 1H).

### Example 1CY

### Preparation of 2-ethyl-N-(1-methyl-1H-pyrazol-3-yl)-4-[4-(S-methylsulfonimidoyl)phenoxy]-2H-indazole-6-carboxamide (1CY)

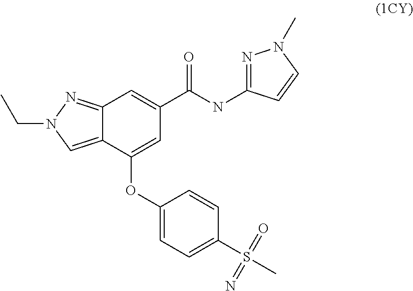

The title compound was prepared by a method analogous to that described for 1CV, using 1-methyl-1H-pyrazol-3-amine.

¹H NMR (400 MHz, 10:1 CHLOROFORM-d/METHANOL-d4) ppm 1.56 (t, 3H), 3.06 (s, 3H), 3.74 (s, 3H), 4.41 (q, 2H), 6.86 (d, 1H), 7.16-7.11 (m, 3H), 7.24 (s, 1H), 7.84-7.86 (m, 1H), 7.86-7.92 (m, 2H), 8.03 (m, 1H). MS (M+1): 439.5.

### Pharmacological Testing

The practice of this invention for the treatment of diseases modulated by the activation of the glucokinase enzyme can be evidenced by activity in at least one of the protocols described herein below.

Representative compounds of this invention were evaluated in biochemical assays (Assay 1 or Assay 2) to characterize their glucokinase activation properties.

The recombinant human glucokinase protein utilized in both assays was prepared and purified as described below.

### Beta Cell Glucokinase His-Tag Growth and Induction Conditions:

BL21(DE3) cells (Invitrogen Corporation, Carlsbad, Calif.) containing pBCGK (C or N H is) vector were grown at 37° C. (in 2XYT) until the OD600 was between 0.6-1.0. Expression was induced by addition of isopropylthiogalactoside to a final concentration of 0.1-0.2 mM to the cells which were then incubated overnight at 23° C. The next day, cells were harvested via centrifugation at 5000 rpm for 15 minutes at 4° C. The cell pellet was stored at −80° C. for future purification.

Beta Cell Glucokinase His-Taq Purification Conditions: A Ni-NTA (Quigan, Germantown, Md.) column (15-50 mL) was used for separation. Two buffers were prepared, 1) a lysis/nickel equilibration and wash buffer and 2) a nickel elution buffer. The lysis/equilibration/wash buffer was prepared as such: 25 mM HEPES buffer at pH 7.5, 250 mM NaCl, 20 mM imidazole, and 14 mM β-mercaptoethanol as final concentrations. The elution buffer was prepared as such: 25 mM HEPES at pH 7.5, 250 mM NaCl, 400 mM imidazole, and 14 mM β-mercaptoethanol as final concentrations. The buffers were each filtered with a 0.22 μm filter prior to use. The cell pellet (1 L culture) was resuspended in 300 mL of the lysis/equilibration buffer. The cells were then lysed (3 times) with a Microfluidics Model 110Y microfluidizer (Microfluidics Corporation, Newton, Mass.). The slurry was centrifuged with a Beckman Coulter Model LE-80K ultracentrifuge (Beckman Coulter, Fullerton, Calif.) at 40,000 rpm for 45 minutes at 4° C. The supernatant was transferred to a chilled flask. A volume of 20 μl was saved for gel analysis. A Pharmacia AKTA (GMI, Inc., Ramsey, Minn.) purification system was used for separation. The prime lines were purged with lysis/equilibration buffer. The Ni-NTA column was equilibrated with 200 mL of the lysis/equilibration buffer at a flow rate of 5 mL/minute. The supernantant was loaded over the column at 4 mL/minute and the flow-through was collected in a flask. The unbound proteins were washed with lysis/equilibration buffer at a flow rate of 5 mL/minute until the ultraviolet reaches baseline. The protein was then eluted from the column with the imidazole elution buffer via imidazole gradient 20 mM to 400 mM over 320 mL. The column was then stripped of any additional protein with 80 mL of the elution buffer. The elution fractions were each 8 mL, for a total yield of 50 samples. Fractions were analyzed by sodium dodecyl sulfate polyacrylamide (SDS-PAGE) and the fractions containing protein of interest were pooled and concentrated to 10 mL using ultrafiltration cell with a 10,000 molecular weight cut-off (MWCO) Millipore membrane (Sigma-Aldrich, St. Louis, Mo.) under nitrogen gas (60 psi). Protein was further purified by size exclusion chromatography (SEC) using a Sedex 75 evaporative light scattering detector (320 mL) (Amersham Pharmacia, Uppsala, Sweden). SEC was equilibrated with 450 mL sizing buffer containing 25 mM HEPES pH 7.0, 50 mM NaCl, and 5 mM dithiothreitol. Concentrated protein was then loaded over SEC and elution with 400 mL sizing buffer was performed overnight at 0.5 mL/minute. The elution fractions were 5 mL each. The fractions were analyzed by SDS-PAGE and protein containing fractions were pooled. Concentration was measured using Bradford Assay/BSA Standard. Purified protein was stored in small aliquots at −80° C.

### Assay 1: Evaluating Activator Potency and Maximum Activation at 5 mM Glucose

Full-length glucokinase (beta cell isoform) was His-tagged at the N-terminus and purified by a Ni column followed by size exclusion chromatography as described above. Glucose was obtained from Calbiochem (San Diego, Calif.) and other reagents were purchased from Sigma-Aldrich (St. Louis, Mo.).

All assays were performed in a Corning 384-well plate using Spectramax PLUS spectrophotometer (Molecular Devices, Sunnyvale, Calif.) at room temperature. The final assay volume was 40 μL. The buffer conditions used in this assay were as follows: 50 mM HEPES, 5 mM glucose, 2.5 mM ATP, 3.5 mM MgCl₂, 0.7 mM NADH, 2 mM dithiothreitol, 1 unit/mL pyruvate kinase/lactate dehydrogenase (PK/LDH), 0.2 mM phosphoenolpyruvate, and 25 mM KCl. The buffer pH was 7.1. The test compound in dimethylsulfoxide solution was added to the buffer and mixed by a plate shaker for 7.5 minutes. The final concentration of dimethylsulfoxide introduced into the assay was 0.25%.

Glucokinase was added to the buffer mixture to initiate the reaction in the presence and absence of compound. The reaction was monitored by absorbance at 340 nm due to the depletion of NADH. The initial reaction velocity was measured by the slope of a linear time course of 0-300 seconds. The percentage of maximum activation was calculated by the following equation:

% Maximum Activation=(Va/Vo−1)×100;

wherein each of Va and Vo is defined as the initial reaction velocity in the presence and absence of the tested compound, respectively.

To determine the EC₅₀ (half maximal effective concentration) and % maximum activation, compounds were serially diluted in dimethylsulfoxide by 3-fold. The glucokinase activities were measured as a function of compound concentrations. The data were fitted to the equation below to obtain the EC₅₀ and % max activation values:

Va/Vo=1+(% max activation/100)/(1+EC₅₀/compound concentration).

The EC₅₀ values for representative examples evaluated are provided in Table 5.

TABLE 5
EC₅₀ of representative examples determined by the method of
Assay 1.

| Example | | Glucokinase |
|---|---|---|
| No. | IUPAC Name | EC50 |
| 1A | 2-methyl-N-(5-methylpyridin-2-yl)-4- | 0.293 μM |
| | {[5-(pyrrolidin-1-ylcarbonyl)pyrazin-2- | (n = 2) |
| | yl]oxy}-2H-indazole-6-carboxamide | |
| 1B | 2-methyl-N-pyrazin-2-yl-4-{[5- | 0.960 μM |
| | (pyrrolidin-1-ylcarbonyl)pyrazin-2- | (n = 2) |
| | yl]oxy}-2H-indazole-6-carboxamide | |
| 1C | 4-[3-fluoro-4-(pyrrolidin-1-ylcarbonyl)- | 1.49 μM |
| | phenoxy]-2-methyl-N-(5- | (n = 1) |
| | methylpyridin-2-yl)-2H-indazole-6- | |
| | carboxamide | |
| 1D | 4-[3-fluoro-4-(pyrrolidin-1-ylcarbonyl)- | 0.735 μM |
| | phenoxy]-2-methyl-N-pyrazin-2-yl- | (n = 2) |
| | 2H-indazole-6-carboxamide | |
| 1E | 4-{[5-(dimethylcarbamoyl)pyrazin-2- | 4.31 μM |
| | yl]oxy}-2-methyl-N-pyrazin-2-yl-2H- | (n = 3) |
| | indazole-6-carboxamide | |
| 1F | 4-{[5-(dimethylcarbamoyl)pyrazin-2- | 2.49 μM |
| | yl]oxy}-2-methyl-N-(5-methylpyrazin- | (n = 6) |
| | 2-yl)-2H-indazole-6-carboxamide | |
| 1G | 4-{[5-(dimethylcarbamoyl)pyrazin-2- | 1.55 μM |
| | yl]oxy}-2-methyl-N-(5-methylpyridin- | (n = 3) |
| | 2-yl)-2H-indazole-6-carboxamide | |
| 1H | 4-{[5-(dimethylcarbamoyl)pyrazin-2- | 2.77 μM |
| | yl]oxy}-N-(5-methoxypyrazin-2-yl)-2- | (n = 3) |
| | methyl-2H-indazole-6-carboxamide | |
| 1I | 2-methyl-N-(5-methylpyrazin-2-yl)-4- | 19.4 μM |
| | {[5-(trifluoromethyl)pyrazin-2-yl]oxy}- | (n = 3) |
| | 2H-indazole-6-carboxamide | |
| 1J | 2-methyl-N-(1-methyl-1H-pyrazol-3- | 22.4 μM |
| | yl)-4-{[5-(trifluoromethyl)pyrazin-2- | (n = 2) |
| | yl]oxy}-2H-indazole-6-carboxamide | |
| 1K | 2-methyl-N-(2-methyl-2H-1,2,3- | 16.1 μM |
| | triazol-4-yl)-4-{[5- | (n = 3) |
| | (trifluoromethyl)pyrazin-2-yl]oxy}-2H- | |
| | indazole-6-carboxamide | |
| 1L | 2-methyl-N-(5-methylpyrazin-2-yl)-4- | 7.81 μM |
| | [4-(methylsulfonyl)phenoxy]-2H- | (n = 2) |
| | indazole-6-carboxamide | |
| 1M | 2-methyl-N-(5-methylpyridin-2-yl)-4- | 2.66 μM |
| | [4-(methylsulfonyl)phenoxy]-2H- | (n = 3) |
| | indazole-6-carboxamide | |
| 1N | 2-methyl-N-(1-methyl-1H-pyrazol-3- | 8.54 μM |
| | yl)-4-[4-(methylsulfonyl)phenoxy]-2H- | (n = 1) |
| | indazole-6-carboxamide | |
| 1O | 4-{[5-(dimethylcarbamoyl)pyrazin-2- | 0.776 μM |
| | yl]oxy}-2-ethyl-N-(5-methylpyridin-2- | (n = 2) |
| | yl)-2H-indazole-6-carboxamide | |
| 1P | 4-{[5-(dimethylcarbamoyl)pyrazin-2- | 2.73 μM |
| | yl]oxy}-2-ethyl-N-(5-methylpyrazin-2- | (n = 2) |
| | yl)-2H-indazole-6-carboxamide | |
| 1Q | 2-ethyl-4-[4- | 2.42 μM |
| | (methylsulfonyl)phenoxy]-N-pyridin-2- | (n = 3) |
| | yl-2H-indazole-6-carboxamide | |
| 1R | 2-ethyl-N-(5-methylpyridin-2-yl)-4-[4- | 1.41 μM |
| | (methylsulfonyl)phenoxy]-2H- | (n = 2) |
| | indazole-6-carboxamide | |
| 1S | 4-[4-(ethylsulfonyl)phenoxy]-2- | 1.34 μM |
| | methyl-N-pyridin-2-yl-2H-indazole-6- | (n = 1) |
| | carboxamide | |
| 1T | 4-[4-(ethylsulfonyl)phenoxy]-2- | 1.95 μM |
| | methyl-N-(5-methylpyrazin-2-yl)-2H- | (n = 1) |
| | indazole-6-carboxamide | |
| 1U | 4-[4-(cyclopropylsulfonyl)phenoxy]-2- | 1.89 μM |
| | methyl-N-(5-methylpyrazin-2-yl)-2H- | (n = 2) |
| | indazole-6-carboxamide | |
| 1V | 4-[4-(cyclopropylsulfonyl)phenoxy]-2- | 1.50 μM |
| | methyl-N-pyridin-2-yl-2H-indazole-6- | (n = 2) |
| | carboxamide | |
| 1W | 4-[4-(cyclopropylsulfonyl)phenoxy]-2- | 1.48 μM |
| | ethyl-N-(5-methylpyrazin-2-yl)-2H- | (n = 1) |
| | indazole-6-carboxamide | |
| 1X | 4-[4-(cyclopropylsulfonyl)phenoxy]-2- | 0.863 μM |
| | ethyl-N-pyridin-2-yl-2H-indazole-6- | (n = 1) |
| | carboxamide | |
| 1Y | 4-{[2-(dimethylcarbamoyl)pyrimidin-5- | 3.34 |
| | yl]oxy}-2-methyl-N-(5-methylpyrazin- | (n = 1) |
| | 2-yl)-2H-indazole-6-carboxamide | |
| 1Z | 4-{[2-(dimethylcarbamoyl)pyrimidin-5- | 1.08 μM |
| | yl]oxy}-2-methyl-N-(5-methylpyridin- | (n = 1) |
| | 2-yl)-2H-indazole-6-carboxamide | |
| 1AA | N-(5-chloropyridin-2-yl)-4-(2- | 0.863 μM |
| | (dimethylcarbamoyl)pyrimidin-5- | (n = 1) |
| | yloxy)-2-methyl-2H-indazole-6- | |
| | carboxamide | |
| 1AB | 4-(2-(dimethylcarbamoyl)pyrimidin-5- | 1.04 μM |
| | yloxy)-2-methyl-N-(5-(trifluoromethyl)- | (n = 1) |
| | pyridin-2-yl)-2H-indazole-6- | |
| | carboxamide | |
| 1AC | 4-{[6-(dimethylcarbamoyl)-5- | 1.63 μM |
| | fluoropyridin-3-yl]oxy}-2-methyl-N-(5- | (n = 3) |
| | methylpyrazin-2-yl)-2H-indazole-6- | |
| | carboxamide | |
| 1AD | 2-ethyl-4-[4-(ethylsulfonyl)phenoxy]- | 0.463 μM |
| | N-(5-methylpyridin-2-yl)-2H-indazole- | (n = 2) |
| | 6-carboxamide | |
| 1AE | 4-(6-(dimethylcarbamoyl)-5- | 0.569 μM |
| | fluoropyridin-3-yloxy)-2-ethyl-N-(5- | (n = 2) |
| | methylpyrazin-2-yl)-2H-indazole-6- | |
| | carboxamide | |

### Assay 2: Evaluating Activator Potency in a Matrix Assay at Multiple Glucose Concentrations

As described by Bebernitz and coworkers (Bebernitz, G. R. et. al., J. Med. Chem. 2009, 52, 6142-6152) the potency of a glucokinase activator and its modulation of the glucokinase enzyme's Km (for glucose) and Vmax can be characterized using a matrix assay wherein multiple combinations of activator and glucose concentrations are simultaneously evaluated. Utilizing an adaptation of this method, representative compounds of the current invention were evaluated at 22 different concentrations and 16 different glucose concentrations in a coupled enzyme assay system that detects glucokinase activity via depletion of β-NADH. The readout is absorbance at 340 nm, and is captured asΔA340/Δtime.

Initially, a 1.0 L volume of assay buffer (at 5 times (5×) final concentration) was prepared utilizing the following reagents (reagent used, formula weight of reagent, 5× concentration of reagent ([5X]), final concentration of reagent after dilution ([Final], and mass of reagent):

HEPES, FW=238.3 g/mol, [5×]=250 mM, [Final]=50 mM, 59.58 g; MgCl₂, FW=203.3 g/mol, [5×]=17.5 mM, [Final]=3.5 mM, 3.56 g; KCl, FW=74.55 g/mol, [5×]=125 mM, [Final]=25 mM, 9.32 g; and BSA, n/a, [5×]=0.5%; [Final]=0.1%.

Compounds are tested against 16 concentrations of glucose. The glucose titration is made at 2 times (2×) the final concentration. The final glucose concentrations used are: 0 mM, 0.05 mM, 0.1 mM, 0.3 mM, 0.625 mM, 1.25 mM, 2.5 mM, 5 mM, 7.5 mm, 10 mM, 15 mM, 20 mM, 40 mM, 60 mM, 80 mM and 100 mm. Plates are stored at 4° C. The glucokinase activator compounds of Formula (I) of the current invention are evaluated at 22 different compound concentrations. The final compound concentrations that are employed are: 0.001 M, 0.0005 M, 0.00025 M, 0.000125 M, 0.0000625 M, 0.00003125 M, 0.000015625 M, 7.81×10⁻⁶ M, 3.91×10⁻⁶ M, 1.95×10⁻⁶ M, 9.77×10⁻⁷ M, 4.88×10⁻⁷ M, 2.44×10⁻⁷ M, 1.22×10⁻⁷ M, 6.10×10⁻⁸ M, 3.05×10⁻⁸ M, 1.53×10⁻⁸ M, 7.63×10⁻⁹ M, 3.81×10⁻⁹ M, 1.91×10⁻⁹ M, 9.54×10⁻¹⁰ M and 4.77×10⁻¹⁰ M.

The assay reagents and final concentrations of the reagents are as follows (reagent, final concentration): GK, 10 nM; Buffer, 1×; ddH₂O; DTT, 2 mM; PEP, 0.8 mM; NADH, 0.7 mM; ATP, 2.5 mM; and PK/LDH, 8 U/mL. The DTT is stored as a frozen 1 M stock. PEP, NADH, and ATP are weighed out as powders. The assay reagents are made up fresh daily, and in two separate components.

The enzyme mix and the substrate mix is outlined as follows. The enzyme mix consists of GK, Buffer (5×), water and DTT. The substrate mix consists of Buffer (5×), water, DTT, PEP, NADH, ATP and PK/LDH. Each mix is made up at 4 times the concentration of the final concentration used.

Assay Protocol: The assay volume is 40 μL per well: 20 μL from glucose, 10 μL from enzyme, and 10 μL from substrate. The final assay plates have 1 μL of compound solution or control in DMSO. When running multiple plates simultaneously on multiple readers, read triplicates on the same reader to decrease variability.

The procedure for carrying out the assay is as follows: Add 20 μL of glucose to each well and centrifuge (1000 rpm, 10 seconds). Add 10 μL of the enzyme mix. Shake plates on plate shaker (900 revolutions per minute) at room temperature (22° C.) for 7 minutes to mix in the compound. Add 10 μL of substrate mix. Shake briefly at room temperature to mix, about 10 seconds and centrifuge to remove bubbles. Examine plate for residual bubbles, and remove them with ethanol vapor. The assay plates are read on a SpectraMax reader (Molecular Devices) using SoftMaxPro 4.8 software. The reader should be configured to read absorbance at wavelength 340 nm, in kinetic mode, read every 30 seconds for 10 minutes. Automix and blanking are off and autocalibrate is set to once.

These data were analyzed by fitting curves to the rates observed for each combination of substrate and activator. This enabled determination of the glucokinase Km (for glucose) and Vmax of at each concentration of activator. Plotting the resulting Km values for each concentration of activator and fitting a curve enabled determination of an intrinsic potency for a given activator determined as the concentration of compound affording a 50% reduction in the enzyme's Km. These intrinsic EC50 values are reported for representative compounds in Table 6.

TABLE 6
EC₅₀ of representative examples determined by the method of
Assay 2.

| Example | | Glucokinase |
|---|---|---|
| No. | IUPAC Name | EC50 |
| 1AF | 4-(5-(dimethylcarbamoyl)pyrazin-2- | 0.532 μM |
| | yloxy)-2-ethyl-N-(5-methoxypyrazin-2- | (n = 2) |
| | yl)-2H-indazole-6-carboxamide | |
| 1AG | 4-(4-(dimethylcarbamoyl)phenoxy)-2- | 0.713 μM |
| | methyl-N-(5-methylpyrazin-2-yl)-2H- | (n = 1) |
| | indazole-6-carboxamide | |
| 1AH | 4-(5-(azetidine-1-carbonyl)pyrazin-2- | 36 μM |
| | yloxy)-2-methyl-N-(2-methyl-2H- | (n = 1) |
| | 1,2,3-triazol-4-yl)-2H-indazole-6- | |
| | carboxamide | |
| 1AI | 4-{4- | 0.431 μM |
| | [(dimethylamino)sulfonyl]phenoxy}-2- | (n = 2) |
| | methyl-N-(5-methylpyrazin-2-yl)-2H- | |
| | indazole-6-carboxamide | |
| 1AJ | 4-{4- | 0.703 μM |
| | [(dimethylamino)sulfonyl]phenoxy}-2- | (n = 2) |
| | methyl-N-(2-methyl-2H-1,2,3-triazol- | |
| | 4-yl)-2H-indazole-6-carboxamide | |
| 1AK | 4-{4- | 0.878 μM |
| | [(methylamino)sulfonyl]phenoxy}-2- | (n = 2) |
| | methyl-N-(5-methylpyrazin-2-yl)-2H- | |
| | indazole-6-carboxamide | |
| 1AL | 4-({6- | 3.17 μM |
| | [(dimethylamino)sulfonyl]pyridin-3- | (n = 1) |
| | yl}oxy)-2-methyl-N-(5-methylpyrazin- | |
| | 2-yl)-2H-indazole-6-carboxamide | |
| 1AM | 4-({6- | 3.88 μM |
| | [(dimethylamino)sulfonyl]pyridin-3- | (n = 1) |
| | yl}oxy)-2-methyl-N-(1-methyl-1H- | |
| | pyrazol-3-yl)-2H-indazole-6- | |
| | carboxamide | |
| 1AN | 2-ethyl-4-({5- | 0.532 μM |
| | [methyl(methylsulfonyl)amino]pyrazin- | (n = 2) |
| | 2-yl}oxy)-N-(5-methylpyridin-2-yl)-2H- | |
| | indazole-6-carboxamide | |
| 1AO | 2-methyl-N-(1-methyl-1H-pyrazol-3- | 1.29 μM |
| | yl)-4-{[6-(methylsulfonyl)pyridin-3- | (n = 2) |
| | yl]oxy}-2H-indazole-6-carboxamide | |
| 1AP | 2-methyl-N-(5-methylpyridin-2-yl)-4- | 1.43 μM |
| | {[6-(methylsulfonyl)pyridin-3-yl]oxy}- | (n = 2) |
| | 2H-indazole-6-carboxamide | |
| 1AQ | 2-methyl-N-(5-methylpyrazin-2-yl)-4- | 5.37 μM |
| | {[6-(methylsulfonyl)pyridin-3-yl]oxy}- | (n = 2) |
| | 2H-indazole-6-carboxamide | |
| 1AR | 2-methyl-N-(5-methylpyridin-2-yl)-4- | 0.545 μM |
| | [4-(methylsulfonyl)-2- | (n = 1) |
| | (trifluoromethyl)phenoxy]-2H- | |
| | indazole-6-carboxamide | |
| 1AS | 4-[4-(aminosulfonyl)phenoxy]-2- | 2.28 μM |
| | methyl-N-(5-methylpyrazin-2-yl)-2H- | (n = 2) |
| | indazole-6-carboxamide | |
| 1AT | 2-ethyl-N-(5-methylpyridin-2-yl)-4-({5- | 4.92 μM |
| | [(methylsulfonyl)amino]pyrazin-2- | (n = 2) |
| | yl}oxy)-2H-indazole-6-carboxamide | |
| 1AU | 4-[4-(aminosulfonyl)phenoxy]-2- | 6.22 μM |
| | methyl-N-(1-methyl-1H-pyrazol-3-yl)- | (n = 2) |
| | 2H-indazole-6-carboxamide | |
| 1AV | 4-[(1,1-dioxido-2,3-dihydro-1- | 0.817 μM |
| | benzothien-5-yl)oxy]-2-methyl-N-(5- | (n = 1) |
| | methylpyridin-2-yl)-2H-indazole-6- | |
| | carboxamide | |
| 1AW | 4-[(1,1-dioxido-2,3-dihydro-1- | 0.986 μM |
| | benzothien-5-yl)oxy]-2-ethyl-N-(5- | (n = 1) |
| | methylpyridin-2-yl)-2H-indazole-6- | |
| | carboxamide | |
| 1AX | 2-ethyl-N-(5-methoxypyrazin-2-yl)-4- | 0.567 μM |
| | [4-(methylsulfonyl)phenoxy]-2H- | (n = 1) |
| | indazole-6-carboxamide | |
| 1AY | 4-({5- | 0.673 μM |
| | [(ethylsulfonyl)(methyl)amino]pyrazin- | (n = 2) |
| | 2-yl}oxy)-2-methyl-N-(5- | |
| | methylpyridin-2-yl)-2H-indazole-6- | |
| | carboxamide | |
| 1AZ | 4-(6-(azetidine-1-carbonyl)-5- | 0.108 μM |
| | fluoropyridin-3-yloxy)-2-ethyl-N-(5- | (n = 4) |
| | methylpyrazin-2-yl)-2H-indazole-6- | |
| | carboxamide | |
| 1BA | 4-(6-(azetidine-1-carbonyl)-5- | 0.191 μM |
| | fluoropyridin-3-yloxy)-2-ethyl-N-(1- | (n = 4) |
| | methyl-1H-pyrazol-3-yl)-2H-indazole- | |
| | 6-carboxamide | |
| 1BB | 4-(6-(dimethylcarbamoyl)-5- | 0.195 μM |
| | fluoropyridin-3-yloxy)-2-ethyl-N- | (n = 10) |
| | (pyrazin-2-yl)-2H-indazole-6- | |
| | carboxamide | |
| 1BC | 4-(6-(azetidine-1-carbonyl)-5- | 0.368 μM |
| | fluoropyridin-3-yloxy)-2-methyl-N-(5- | (n = 2) |
| | methylpyrazin-2-yl)-2H-indazole-6- | |
| | carboxamide | |
| 1BD | 4-(2-(dimethylcarbamoyl)pyrimidin-5- | 0.383 μM |
| | yloxy)-2-ethyl-N-(5-methylpyridin-2- | (n = 2) |
| | yl)-2H-indazole-6-carboxamide | |
| 1BE | 4-(6-(dimethylcarbamoyl)-5- | 0.456 μM |
| | fluoropyridin-3-yloxy)-N-(5- | (n = 2) |
| | methoxypyrazin-2-yl)-2-methyl-2H- | |
| | indazole-6-carboxamide | |
| 1BG | N-(5-ethoxypyrazin-2-yl)-2-ethyl-4-[4- | 0.203 μM |
| | (methylsulfonyl)phenoxy]-2H- | (n = 2) |
| | indazole-6-carboxamide | |
| 1BH | 2-ethyl-N-(5-ethylpyrazin-2-yl)-4-[4- | 0.536 μM |
| | (methylsulfonyl)phenoxy]-2H- | (n = 1) |
| | indazole-6-carboxamide | |
| 1BI | 4-[4-(aminosulfonyl)phenoxy]-N-(5- | 49.9 μM |
| | ethoxypyrazin-2-yl)-2-ethyl-2H- | (n = 1) |
| | indazole-6-carboxamide | |
| 1BK | 4-{4- | 1.01 μM |
| | [(dimethylamino)sulfonyl]phenoxy}-2- | (n = 1) |
| | methyl-N-(1-methyl-1H-pyrazol-3-yl)- | |
| | 2H-indazole-6-carboxamide | |
| 1BL | 2-methyl-4-{4- | 1.80 μM |
| | [(methylamino)sulfonyl]phenoxy}-N- | (n = 2) |
| | (1-methyl-1H-pyrazol-3-yl)-2H- | |
| | indazole-6-carboxamide | |
| 1BM | 2-ethyl-N-(1-ethyl-1H-pyrazol-3-yl)-4- | 1.90 μM |
| | [4-(methylsulfonyl)phenoxy]-2H- | (n = 1) |
| | indazole-6-carboxamide | |
| 1BN | N-(5-methoxypyrazin-2-yl)-2-methyl- | 0.684 μM |
| | 4-{4- | (n = 2) |
| | [(methylamino)sulfonyl]phenoxy}-2H- | |
| | indazole-6-carboxamide | |
| 1BO | 2-methyl-N-(1-methyl-1H-pyrazol-3- | 0.250 μM |
| | yl)-4-(5-(pyrrolidine-1- | (n = 2) |
| | carbonyl)pyrazin-2-yloxy)-2H- | |
| | indazole-6-carboxamide | |
| 1BP | 2-ethyl-N-(5-methylpyrazin-2-yl)-4-(5- | 0.290 μM |
| | (pyrrolidine-1-carbonyl)pyrazin-2- | (n = 2) |
| | yloxy)-2H-indazole-6-carboxamide | |
| 1BQ | 4-(5-(azetidine-1-carbonyl)pyrazin-2- | 0.699 μM |
| | yloxy)-2-ethyl-N-(1-methyl-1H- | (n = 2) |
| | pyrazol-3-yl)-2H-indazole-6- | |
| | carboxamide | |
| 1BR | 4-(5-(azetidine-1-carbonyl)pyrazin-2- | 0.790 μM |
| | yloxy)-2-ethyl-N-(5-methylpyrazin-2- | (n = 3) |
| | yl)-2H-indazole-6-carboxamide | |
| 1BS | 4-(5-(azetidine-1-carbonyl)pyrazin-2- | 0.798 μM |
| | yloxy)-2-methyl-N-(1-methyl-1H- | (n = 3) |
| | pyrazol-3-yl)-2H-indazole-6- | |
| | carboxamide | |
| 1BT | 4-(5-(azetidine-1-carbonyl)pyrazin-2- | 0.979 μM |
| | yloxy)-2-isopropyl-N-(1-methyl-1H- | (n = 3) |
| | pyrazol-3-yl)-2H-indazole-6- | |
| | carboxamide | |
| 1BU | 4-(5-(dimethylcarbamoyl)pyrazin-2- | 1.05 μM |
| | yloxy)-2-ethyl-N-(1-methyl-1H- | (n = 2) |
| | pyrazol-3-yl)-2H-indazole-6- | |
| | carboxamide | |
| 1BV | 4-(5-(dimethylcarbamoyl)pyrazin-2- | 1.59 μM |
| | yloxy)-2-isopropyl-N-(1-methyl-1H- | (n = 3) |
| | pyrazol-3-yl)-2H-indazole-6- | |
| | carboxamide | |
| 1BW | 2-methyl-N-(2-methyl-2H-1,2,3- | 4.64 μM |
| | triazol-4-yl)-4-(5-(pyrrolidine-1- | (n = 2) |
| | carbonyl)pyrazin-2-yloxy)-2H- | |
| | indazole-6-carboxamide | |
| 1BX | 4-(5-(azetidine-1-carbonyl)pyrazin-2- | 15.1 μM |
| | yloxy)-2-ethyl-N-(2-methyl-2H-1,2,3- | (n = 1) |
| | triazol-4-yl)-2H-indazole-6- | |
| | carboxamide | |
| 1BY | 4-(5-(azetidine-1-carbonyl)pyrazin-2- | 17.5 μM |
| | yloxy)-2-isopropyl-N-(2-methyl-2H- | (n = 2) |
| | 1,2,3-triazol-4-yl)-2H-indazole-6- | |
| | carboxamide | |
| 1BZ | 4-(5-(dimethylcarbamoyl)pyrazin-2- | 25.1 μM |
| | yloxy)-2-ethyl-N-(2-methyl-2H-1,2,3- | (n = 2) |
| | triazol-4-yl)-2H-indazole-6- | |
| | carboxamide | |
| 1CA | 2-ethyl-4-[4- | 8.52 μM |
| | (methylsulfonyl)phenoxy]-N-(2- | (n = 2) |
| | methyl-2H-1,2,3-triazol-4-yl)-2H- | |
| | indazole-6-carboxamide | |
| 1CB | 2-ethyl-N-(5-methylpyrazin-2-yl)-4-[4- | 0.537 μM |
| | (methylsulfonyl)phenoxy]-2H- | (n = 2) |
| | indazole-6-carboxamide | |
| 1CC | 2-ethyl-N-(1-methyl-1H-pyrazol-3-yl)- | 1.21 μM |
| | 4-[4-(methylsulfonyl)phenoxy]-2H- | (n = 1) |
| | indazole-6-carboxamide | |
| 1CD | N-(5-carbamoylpyridin-2-yl)-2-ethyl-4- | 0.261 μM |
| | [4-(methylsulfonyl)phenoxy]-2H- | (n = 3) |
| | indazole-6-carboxamide | |
| 1CE | N-[5-(2-amino-2-oxoethyl)pyridin-2- | 0.558 μM |
| | yl]-2-ethyl-4[4- | (n = 3) |
| | (methylsulfonyl)phenoxy]-2H- | |
| | indazole-6-carboxamide | |
| 1CF | 2-ethyl-N-(5-fluoropyridin-2-yl)-4-[4- | 0.714 μM |
| | (methylsulfonyl)phenoxy]-2H- | (n = 1) |
| | indazole-6-carboxamide | |
| 1CG | N-[1-(2-amino-2-oxoethyl)-1H- | 1.49 μM |
| | pyrazol-3-yl]-2-ethyl-4-[4- | (n = 1) |
| | (methylsulfonyl)phenoxy]-2H- | |
| | indazole-6-carboxamide | |
| 1CI | 2-ethyl-N-(5-fluoropyridin-2-yl)-4-[4- | 0.714 μM |
| | (methylsulfonyl)phenoxy]-2H- | (n = 1) |
| | indazole-6-carboxamide | |
| 1CJ | 2-methyl-4-[4- | 0.282 μM |
| | (methylsulfonyl)phenoxy]-N-(1- | (n = 1) |
| | methyl-1H-1,2,3-triazol-4-yl)-2H- | |
| | indazole-6-carboxamide | |
| 1CK | methyl 6-[({2-ethyl-4-[4- | 0.396 μM |
| | (methylsulfonyl)phenoxy]-2H-indazol- | (n = 2) |
| | 6-yl}carbonyl)amino]nicotinate | |
| 1CL | 2-ethyl-4-[4- | 0.919 μM |
| | (methylsulfonyl)phenoxy]-N-1,2,4- | (n = 1) |
| | thiadiazol-5-yl-2H-indazole-6- | |
| | carboxamide | |
| 1CM | 4-(5-acetamidopyrazin-2-yloxy)-2- | 2.45 μM |
| | ethyl-N-(5-methylpyridin-2-yl)-2H- | (n = 1) |
| | indazole-6-carboxamide | |
| 1CN | 2-methyl-4-(5-(N- | 2.32 μM |
| | methylacetamido)pyrazin-2-yloxy)-N- | (n = 1) |
| | (5-methylpyridin-2-yl)-2H-indazole-6- | |
| | carboxamide | |
| 1CO | 4-(5-carbamoylpyrazin-2-yloxy)-2- | 1.88 μM |
| | ethyl-N-(5-methylpyridin-2-yl)-2H- | (n = 2) |
| | indazole-6-carboxamide | |
| 1CP | 2-ethyl-N-(5-methylpyridin-2-yl)-4-(5- | 2.28 μM |
| | (morpholine-4-carbonyl)pyrazin-2- | (n = 1) |
| | yloxy)-2H-indazole-6-carboxamide | |
| 1CQ | 4-[4-(aminosulfonyl)phenoxy]-2-ethyl- | 2.88 μM |
| | N-(1-methyl-1H-pyrazol-3-yl)-2H- | (n = 1) |
| | indazole-6-carboxamide | |
| 1CR | 4-[4-(aminosulfonyl)phenoxy]-2-ethyl- | 0.586 μM |
| | N-(5-methylpyrazin-2-yl)-2H-indazole- | (n = 1) |
| | 6-carboxamide | |
| 1CS | 4-[4-(aminosulfonyl)phenoxy]-2-ethyl- | 0.418 μM |
| | N-(5-methoxypyrazin-2-yl)-2H- | (n = 1) |
| | indazole-6-carboxamide | |
| 1CT | 4-[4-(aminosulfonyl)phenoxy]-2-ethyl- | 1.82 μM |
| | N-(5-fluoropyridin-2-yl)-2H-indazole- | (n = 2) |
| | 6-carboxamide | |
| 1CU | 4-[4-(aminosulfonyl)phenoxy]-2-ethyl- | 1.37 μM |
| | N-(5-ethylpyrazin-2-yl)-2H-indazole-6- | (n = 1) |
| | carboxamide | |
| 1CV | 2-ethyl-N-(5-methylpyridin-2-yl)-4-[4- | 0.715 μM |
| | (S-methylsulfonimidoyl)phenoxy]-2H- | (n = 2) |
| | indazole-6-carboxamide | |
| 1CW | 2-ethyl-N-(5-methylpyridin-2-yl)-4-[4- | 0.555 μM |
| | (S-methylsulfonimidoyl)phenoxy]-2H- | (n = 3) |
| | indazole-6-carboxamide | |
| 1CX | 2-ethyl-N-(5-methylpyridin-2-yl)-4-[4- | 1.37 μM |
| | (S-methylsulfonimidoyl)phenoxy]-2H- | (n = 2) |
| | indazole-6-carboxamide | |
| 1CY | 2-ethyl-N-(1-methyl-1H-pyrazol-3-yl)- | 2.47 μM |
| | 4-[4-(S- | (n = 1) |
| | methylsulfonimidoyl)phenoxy]-2H- | |
| | indazole-6-carboxamide | |

We claim:

1. A compound of Formula (I) 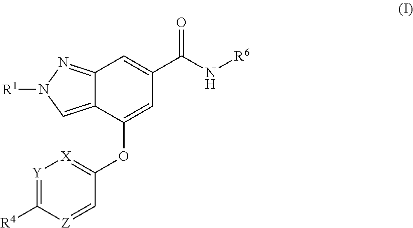
or a pharmaceutically acceptable salt thereof,
wherein R¹ is (C₁-C₄)alkyl;
X is C—R² or N, where R² is hydrogen, halo, or methyl;
Y is C—R³ or N, where R³ is hydrogen, halo, or methyl;
R⁴ is —CF₃, —SO₂R^{4a}, —C(O)NR^{4b}R^{4c}, —SO₂NR^{4b}R^{4c}, —N(R^{4b})SO₂R^{4a}, —N(R^{4b})C(O)NR^{4b}R^{4c} or —S(N)(O)R^{4b};
Z is C—R⁵ or N, where R⁵ is hydrogen, halo, or methyl;
R⁶ is a 5- to 6-membered heteroaryl containing 1 to 3 nitrogen atoms optionally substituted with a substituent selected from the group consisting of (C₁-C₃)alkyl, (C₁-C₃)alkoxy, cyano, halo, —CO₂H, —CO₂(C₁-C₃)alkyl, —NH₂, —NH(C₁-C₄)alkyl, —N((C₁-C₃)alkyl)₂, —CF₃, —C(O)NH(C₁-C₃)alkyl, —C(O)N((C₁-C₃)alkyl)₂ and —C(R^{4b}R^{4c})C(O)N((C₁-C₃)alkyl)₂;
R^{4a} is (C₁-C₃)alkyl or cyclopropyl; and R^{4b} and R^{4c} are at each occurrence independently (C₁-C₃)alkyl or hydrogen or taken together with the nitrogen to which they are attached form an azetidine, pyrrolidine or piperidine ring.

2. The compound of claim 1 or a pharmaceutically acceptable salt thereof, wherein
R¹ is methyl or ethyl;
X is N or C—R², where R² is hydrogen or fluoro;
Y is C—R³, where R³ is hydrogen or fluoro;
R⁴ is —CF₃, —SO₂R^{4a}, —C(O)NR^{4b}R^{4c}, where R^{4a} is methyl, ethyl or cyclopropyl, and R^{4b} and R^{4c} are both methyl or taken together with the nitrogen to which they are attached form a pyrrolidine ring;
Z is N or C—R⁵, where R⁵ is hydrogen or fluoro; and
R⁶ is a 5- to 6-membered heteroaryl selected from pyridin-2-yl, pyrazin-2-yl, 1H-pyrazol-3-yl, or 1,2,3-triazol-4-yl, wherein said heteroaryl is optionally substituted with methyl or methoxy.

3. The compound of claim 1 or a pharmaceutically acceptable salt thereof, wherein
R¹ is methyl or ethyl;
X and Z are both N;
Y is C—R³, where R³ is hydrogen or fluoro;
R⁴ is —CF₃, —SO₂R^{4a}, —C(O)NR^{4b}R^{4c}, where R^{4a} is methyl, ethyl or cyclopropyl, and R^{4b} and R^{4c} are both methyl or taken together forms a pyrrolidine ring;
Z is N or C—R⁵, where R⁵ is hydrogen or fluoro; and
R⁶ is a 5- to 6-membered heteroaryl selected from pyridin-2-yl, pyrazin-2-yl, 1H-pyrazol-3-yl, or 1,2,3-triazol-4-yl, wherein said heteroaryl is optionally substituted with methyl or methoxy.

4. A compound of claim 3 selected from the group consisting of
2-methyl-N-(5-methylpyridin-2-yl)-4-{[5-(pyrrolidin-1-ylcarbonyl)pyrazin-2-yl]oxy}-2H-indazole-6-carboxamide;
2-methyl-N-pyrazin-2-yl-4-{[5-(pyrrolidin-1-ylcarbonyl)pyrazin-2-yl]oxy}-2H-indazole-6-carboxamide;
4-{[5-(dimethylcarbamoyl)pyrazin-2-yl]oxy}-2-methyl-N-pyrazin-2-yl-2H-indazole-6-carboxamide;
4-{[5-(dimethylcarbamoyl)pyrazin-2-yl]oxy}-2-methyl-N-(5-methylpyrazin-2-yl)-2H-indazole-6-carboxamide;
4-{[5-(dimethylcarbamoyl)pyrazin-2-yl]oxy}-2-methyl-N-(5-methylpyridin-2-yl)-2H-indazole-6-carboxamide;
4-{[5-(dimethylcarbamoyl)pyrazin-2-yl]oxy}-N-(5-methoxypyrazin-2-yl)-2-methyl-2H-indazole-6-carboxamide;
2-methyl-N-(5-methylpyrazin-2-yl)-4-{[5-(trifluoromethyl)pyrazin-2-yl]oxy}-2H-indazole-6-carboxamide;
2-methyl-N-(1-methyl-1H-pyrazol-3-yl)-4-{[5-(trifluoromethyl)pyrazin-2-yl]oxy}-2H-indazole-6-carboxamide;
2-methyl-N-(2-methyl-2H-1,2,3-triazol-4-yl)-4-{[5-(trifluoromethyl)pyrazin-2-yl]oxy}-2H-indazole-6-carboxamide;
4-{[5-(dimethylcarbamoyl)pyrazin-2-yl]oxy}-2-ethyl-N-(5-methylpyridin-2-yl)-2H-indazole-6-carboxamide; and
4-{[5-(dimethylcarbamoyl)pyrazin-2-yl]oxy}-2-ethyl-N-(5-methylpyrazin-2-yl)-2H-indazole-6-carboxamide; or a pharmaceutically acceptable salt thereof.

5. A compound of claim 4 selected from the group consisting of
4-{[5-(dimethylcarbamoyl)pyrazin-2-yl]oxy}-2-methyl-N-pyrazin-2-yl-2H-indazole-6-carboxamide;
4-{[5-(dimethylcarbamoyl)pyrazin-2-yl]oxy}-2-methyl-N-(5-methylpyrazin-2-yl)-2H-indazole-6-carboxamide;
4-{[5-(dimethylcarbamoyl)pyrazin-2-yl]oxy}-2-methyl-N-(5-methylpyridin-2-yl)-2H-indazole-6-carboxamide;
4-{[5-(dimethylcarbamoyl)pyrazin-2-yl]oxy}-2-ethyl-N-(5-methylpyridin-2-yl)-2H-indazole-6-carboxamide; and
4-{[5-(dimethylcarbamoyl)pyrazin-2-yl]oxy}-2-ethyl-N-(5-methylpyrazin-2-yl)-2H-indazole-6-carboxamide; or a pharmaceutically acceptable salt thereof.

6. The compound of claim 1 or a pharmaceutically acceptable salt thereof, wherein
R¹ is methyl or ethyl;
X is C—R², where R² is hydrogen or fluoro;
Y is C—R³, where R³ is hydrogen or fluoro;
R⁴ is —CF₃, —SO₂R^{4a}, —C(O)NR^{4b}R^{4c}, where R^{4a} is methyl, ethyl or cyclopropyl, and R^{4b} and R^{4c} are both methyl or taken together with the nitrogen to which they are attached form a pyrrolidine ring;
Z is C—R⁵, where R⁵ is hydrogen or fluoro; and
R⁶ is a 5- to 6-membered heteroaryl selected from pyridin-2-yl, pyrazin-2-yl, 1H-pyrazol-3-yl, or 1,2,3-triazol-4-yl, wherein said heteroaryl is optionally substituted with methyl or methoxy.

7. The compound of claim 6 selected from the group consisting of
4-[3-fluoro-4-(pyrrolidin-1-ylcarbonyl)-phenoxy]-2-methyl-N-(5-methylpyridin-2-yl)-2H-indazole-6-carboxamide;
4-[3-fluoro-4-(pyrrolidin-1-ylcarbonyl)-phenoxy]-2-methyl-N-pyrazin-2-yl-2H-indazole-6-carboxamide;
2-methyl-N-(5-methylpyrazin-2-yl)-4-[4-(methylsulfonyl)phenoxy]-2H-indazole-6-carboxamide;
2-methyl-N-(5-methylpyridin-2-yl)-4-[4-(methylsulfonyl)phenoxy]-2H-indazole-6-carboxamide;
2-methyl-N-(1-methyl-1H-pyrazol-3-yl)-4-[4-(methylsulfonyl)phenoxy]-2H-indazole-6-carboxamide;
2-ethyl-4-[4-(methylsulfonyl)phenoxy]-N-pyridin-2-yl-2H-indazole-6-carboxamide;
2-ethyl-N-(5-methylpyridin-2-yl)-4-[4-(methylsulfonyl)phenoxy]-2H-indazole-6-carboxamide;
4-[4-(ethylsulfonyl)phenoxy]-2-methyl-N-pyridin-2-yl-2H-indazole-6-carboxamide;
4-[4-(ethylsulfonyl)phenoxy]-2-methyl-N-(5-methylpyrazin-2-yl)-2H-indazole-6-carboxamide;
4-[4-(cyclopropylsulfonyl)phenoxy]-2-methyl-N-(5-methylpyrazin-2-yl)-2H-indazole-6-carboxamide;
4-[4-(cyclopropylsulfonyl)phenoxy]-2-methyl-N-pyridin-2-yl-2H-indazole-6-carboxamide;
4-[4-(cyclopropylsulfonyl)phenoxy]-2-ethyl-N-(5-methylpyrazin-2-yl)-2H-indazole-6-carboxamide;
4-[4-(cyclopropylsulfonyl)phenoxy]-2-ethyl-N-pyridin-2-yl-2H-indazole-6-carboxamide; and
2-ethyl-4-[4-(ethylsulfonyl)phenoxy]-N-(5-methylpyridin-2-yl)-2H-indazole-6-carboxamide; or a pharmaceutically acceptable salt thereof.

8. The compound of claim 7 selected from the group consisting of
2-methyl-N-(5-methylpyrazin-2-yl)-4-[4-(methylsulfonyl)phenoxy]-2H-indazole-6-carboxamide;
2-methyl-N-(5-methylpyridin-2-yl)-4-[4-(methylsulfonyl)phenoxy]-2H-indazole-6-carboxamide;
2-methyl-N-(1-methyl-1H-pyrazol-3-yl)-4-[4-(methylsulfonyl)phenoxy]-2H-indazole-6-carboxamide;
2-ethyl-4-[4-(methylsulfonyl)phenoxy]-N-pyridin-2-yl-2H-indazole-6-carboxamide;
2-ethyl-N-(5-methylpyridin-2-yl)-4-[4-(methylsulfonyl)phenoxy]-2H-indazole-6-carboxamide;
4-[4-(ethylsulfonyl)phenoxy]-2-methyl-N-pyridin-2-yl-2H-indazole-6-carboxamide;
4-[4-(ethylsulfonyl)phenoxy]-2-methyl-N-(5-methylpyrazin-2-yl)-2H-indazole-6-carboxamide; and
2-ethyl-4-[4-(ethylsulfonyl)phenoxy]-N-(5-methylpyridin-2-yl)-2H-indazole-6-carboxamide; or a pharmaceutically acceptable salt thereof.

9. The compound of claim 1 or a pharmaceutically acceptable salt thereof, wherein
R¹ is methyl or ethyl;
X is C—R², where R² is hydrogen;
Y and Z are both N;
R⁴ is —CF₃, —SO₂R^{4a}, —C(O)NR^{4b}R^{4c}, where R^{4a} is methyl, ethyl or cyclopropyl, and R^{4b} and R^{4c} are both methyl or taken together forms a pyrrolidine ring; and
R⁶ is a 5- to 6-membered heteroaryl selected from pyridin-2-yl, pyrazin-2-yl, 1H-pyrazol-3-yl, or 1,2,3-triazol-4-yl, wherein said heteroaryl is optionally substituted with methyl or methoxy.

10. The compound of claim 9 selected from the group consisting of
4-{[2-(dimethylcarbamoyl)pyrimidin-5-yl]oxy}-2-methyl-N-(5-methylpyrazin-2-yl)-2H-indazole-6-carboxamide;
4-{[2-(dimethylcarbamoyl)pyrimidin-5-yl]oxy}-2-methyl-N-(5-methylpyridin-2-yl)-2H-indazole-6-carboxamide;
N-(5-chloropyridin-2-yl)-4-(2-(dimethylcarbamoyl)pyrimidin-5-yloxy)-2-methyl-2H-indazole-6-carboxamide; and
4-(2-(dimethylcarbamoyl)pyrimidin-5-yloxy)-2-methyl-N-(5-(trifluoromethyl)-pyridin-2-yl)-2H-indazole-6-carboxamide; or a pharmaceutically acceptable salt thereof.

11. The compound of claim 1 or a pharmaceutically acceptable salt thereof, wherein
R¹ is methyl or ethyl;
X is C—R², where R² is hydrogen or fluoro;
Y is C—R³, where R³ is hydrogen or fluoro;
R⁴ is —CF₃, —SO₂R^{4a}, —C(O)NR^{4b}R^{4c}, where R^{4a} is methyl, ethyl or cyclopropyl, and R^{4b} and R^{4c} are both methyl or taken together forms a pyrrolidine ring;
Z is N; and
R⁶ is a 5- to 6-membered heteroaryl selected from pyridin-2-yl, pyrazin-2-yl, 1H-pyrazol-3-yl, or 1,2,3-triazol-4-yl, wherein said heteroaryl is optionally substituted with methyl or methoxy.

12. The compound of claim 11 selected from the group consisting of
4-{[6-(dimethylcarbamoyl)-5-fluoropyridin-3-yl]oxy}-2-methyl-N-(5-methylpyrazin-2-yl)-2H-indazole-6-carboxamide; and
4-(6-(dimethylcarbamoyl)-5-fluoropyridin-3-yloxy)-2-ethyl-N-(5-methylpyrazin-2-yl)-2H-indazole-6-carboxamide; or a pharmaceutically acceptable salt thereof.

13. A compound selected from
4-(6-(azetidine-1-carbonyl)-5-fluoropyridin-3-yloxy)-2-ethyl-N-(5-methylpyrazin-2-yl)-2H-indazole-6-carboxamide;
N-(5-ethoxypyrazin-2-yl)-2-ethyl-4-[4-(methylsulfonyl)phenoxy]-2H-indazole-6-carboxamide;
2-ethyl-N-(5-ethylpyrazin-2-yl)-4-[4-(methylsulfonyl)phenoxy]-2H-indazole-6-carboxamide;
4-[4-(aminosulfonyl)phenoxy]-N-(5-ethoxypyrazin-2-yl)-2-ethyl-2H-indazole-6-carboxamide; and
2-ethyl-N-(5-methylpyrazin-2-yl)-4-[4-(methylsulfonyl)phenoxy]-2H-indazole-6-carboxamide; or a pharmaceutically acceptable salt thereof.

14. A pharmaceutical composition comprising (i) a therapeutically effective amount of a compound of any one of claims 1 through 13 or a pharmaceutically acceptable salt thereof; and (ii) a pharmaceutically acceptable excipient, diluent, or carrier.

15. A method for treating obesity and obesity-related disorders or for treating or delaying the progression or onset of Type 2 diabetes and diabetes-related disorders in animals comprising the step of administering to an animal in need of such treatment a therapeutically effective amount of a compound of any one of claims 1 through 13 or a pharmaceutically acceptable salt thereof.
